(19) United States
(12) Patent Application Publication
(10) Pub. No.: US 2006/0051405 A1
(43) Pub. Date: Mar. 9, 2006

(54) Compositions for the delivery of therapeutic agents and uses thereof

(75) Inventors: Ian MacLachlan, Vancouver (CA); Lloyd Jeffs, Delta (CA)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Protiva Biotherapeutics, Inc., Burnaby (CA)

(21) Appl. No.: 11/185,447

(22) Filed: Jul. 19, 2005

Related U.S. Application Data
(60) Provisional application No. 60/656,770, filed on Feb. 25, 2005.
(60) Provisional application No. 60/589,363, filed on Jul. 19, 2004.

(51) Int. Cl. A61K 48/00; A61K 9/127; C12N 15/88

(52) U.S. Cl. 424/450; 435/458; 514/44

(57) ABSTRACT

The present invention provides drug delivery vehicles comprising polytheylyene-lipid conjugates (PEG-lipid), wherein the circulation lifetime and biodistribution of the drug delivery vehicles are regulated by the PEG-lipid. More particularly, the present invention provides liposomes, SNALP and SPLP comprising such PEG-lipid conjugates, and methods of using such compositions to selectively target a tumor site or other tissue of interest (e.g., liver, lung, spleen, etc.).

### CROSS-REFERENCES TO RELATED APPLICATIONS

This application is related to U.S. Provisional Application No. 60/589,363, filed Jul. 19, 2004, the disclosures of which is hereby incorporated by reference in its entirety for all purposes.

### BACKGROUND OF THE INVENTION

An effective and safe gene delivery system is required for gene therapy to be clinically useful. Viral vectors are relatively efficient gene delivery systems, but suffer from a variety of limitations, such as the potential for reversion to the wild type as well as immune response concerns. As a result, nonviral gene delivery systems are receiving increasing attention (Worgall et al., Human Gene Therapy, 8:37-44 (1997); Peeters et al., Human Gene Therapy, 7:1693-1699 (1996); Yei et al., Gene Therapy, 1:192-200 (1994); Hope et al., Molecular Membrane Biology, 15:1-14 (1998)). Plasmid DNA-cationic liposome complexes are currently the most commonly employed nonviral gene delivery vehicles (Felgner, Scientific American, 276:102-106 (1997); Chonn et al., Current Opinion in Biotechnology, 6:698-708 (1995)). However, complexes are large, poorly defined systems that are not suited for systemic applications and can elicit considerable toxic side effects (Harrison et al., Biotechniques, 19:816-823 (1995); Huang et al., Nature Biotechnology, 15:620-621 (1997); Templeton et al., Nature Biotechnology, 15:647-652 (1997); Hofland et al., Pharmaceutical Research, 14:742-749 (1997)).

Recent work has shown that plasmid DNA can be encapsulated in small (˜70 nm diameter) “stabilized plasmid-lipid particles” (SPLP) that consist of a single plasmid encapsulated within a bilayer lipid vesicle (Wheeler et al., Gene Therapy, 6:271-281 (1999)). These SPLPs typically contain the “fusogenic” lipid dioleoylphosphatidyl-ethanolamine (DOPE), low levels of cationic lipid, and are stabilized in aqueous media by the presence of a poly(ethylene glycol) (PEG) coating. SPLP have systemic application as they exhibit extended circulation lifetimes following intravenous (i.v.) injection, accumulate preferentially at distal tumor sites due to the enhanced vascular permeability in such regions, and can mediate transgene expression at these tumor sites. The levels of transgene expression observed at the tumor site following i.v. injection of SPLP containing the luciferase marker gene are superior to the levels that can be achieved employing plasmid DNA-cationic liposome complexes (lipoplexes) or naked DNA. Still, improved levels of expression may be required for optimal therapeutic benefit in some applications (see, e.g., Monck et al., J. Drug Targ., 7:439-452 (2000)).

Typically, both liposomes and SPLPs comprise PEG-lipid conjugates. The PEG-lipid conjugate provides the liposome or particle with a PEG coating that both stabilizes the particle and shields the surface positive charge, preventing rapid systemic clearance. Therefore, it is desirable to identify PEG-lipids that allow for the selective targeting of liposomal or SPLP drug delivery systems. The present invention addresses this and other needs.

### SUMMARY OF THE INVENTION

It has now been discovered that by controlling the length of the alkyl or acyl chains of the lipid portion of the PEG-lipid conjugate of a liposomal, SNALP or SPLP drug delivery system, one can control the circulation lifetime of the drug delivery system and, in turn, the biodistribution of the drug delivery vehicle. More particularly, by controlling the length of the alkyl or acyl chains of the lipid portion of the PEG-lipid conjugate, one can preferentially target the liposomal, SNALP or SPLP drug delivery system to a tumor or other target tissue of interest (e.g., the liver, lung, etc.). For instance, PEG-lipid conjugates having longer, more securely fastened anchors will confer greater stability and extended circulation lifetimes of the liposomal, SNALP or SPLP drug delivery systems. Longer circulating liposomal, SNALP or SPLP drug delivery systems are able to take advantage of “passive targeting,” whereby fenestrations in the tumor vasculature lead to greater accumulation at the tumor site. Conversely, PEG-lipid conjugates having shorter, less securely fastened anchors will confer less stability and shorter circulation lifetimes of the liposomal, SNALP or SPLP drug delivery systems. Shorter circulating liposomal, SNALP or SPLP drug delivery systems preferentially accumulate in the liver. Thus, by controlling the length of the alkyl or acyl chains of the lipid of the PEG-lipid conjugate, one can modulate the time that the PEG-lipid conjugate remains associated with the bilayer and, in turn, the biodistribution of the liposomal, SNALP or SPLP drug delivery vehicle.

As such, in one embodiment, the present invention provides a method of introducing a nucleic acid into a tumor cell, the method comprising contacting the tumor cell with a nucleic acid-lipid particle comprising a cationic lipid, a noncationic lipid, a PEG-lipid conjugate, and a nucleic acid, wherein the alkyl or acyl chains of the lipid portion of the PEG-lipid conjugate comprise from 16 to 20 carbon atoms. The use of such longer chain PEG-lipid conjugates results in the preferential accumulation of the drug delivery vehicle at the tumor site. Moreover, when the drug delivery vehicle is a SPLP, the use of such longer chain PEG-lipid conjugates results in higher transfection efficiencies than shorter chain PEG-lipid conjugates.

In another embodiment, the present invention provides a method of introducing a nucleic acid to the lung of a mammal, the method comprising administering to the mammal a nucleic acid-lipid particle comprising a cationic lipid, a noncationic lipid, a PEG-lipid conjugate, and a nucleic acid, wherein the alkyl or acyl chains of the lipid portion of the PEG-lipid conjugate comprise from 16 to 20 carbon atoms

In another embodiment, the present invention provides a method of introducing a nucleic acid to the liver of a mammal, said method comprising administering to the mammal a nucleic acid-lipid particle comprising a cationic lipid, a noncationic lipid, a PEG-lipid conjugate, and a nucleic acid, wherein the alkyl or acyl chains of the lipid portion of the PEG-lipid conjugate comprise from 8 to 14 carbon atoms. Similarly, the present invention provides a method of introducing a nucleic acid to the spleen of a mammal, the method comprising administering to the mammal a nucleic acid-lipid particle comprising a cationic lipid, a noncationic lipid, a PEG-lipid conjugate, and a nucleic acid, wherein the alkyl or acyl chains of the lipid portion of the PEG-lipid conjugate comprise from 8 to 14 carbon atoms.

Quite importantly, it has also been surprisingly discovered that the methods and compositions of the present invention can advantageously be used to preferentially deliver siRNA to a tumor site or other target tissue of interest. Again, longer chain PEG-lipid conjugates (e.g., C16, C18 or C20) result in the preferential delivery of the siRNA to a tumor site or the lung. Conversely, shorter chain PEG-lipid conjugates (e.g., C8, C12 or C14) result in the preferential delivery of the siRNA to the liver or spleen.

Other features, objects and advantages of the invention and its preferred embodiments will become apparent from the detailed description, examples, claims and figures that follow.

### BRIEF DESCRIPTION OF THE DRAWINGS

FIG. 1 illustrates the chemical structures of the PEG-lipids incorporated into SPLP (a) PEG-Ceramides (b) PEG-S-Diacylglycerols. DMG=Dimyristoylglycerol, DPG=Dipalmitoylglycerol, DSG=Distearoylglycerol.

FIG. 2 illustrates an exchange assay examining the rate of diffusion of the different PEG-lipids from LUV by measuring respective rates of fusion in the presence of a PEG-lipid sink. Fluorescence resonance energy transfer labels were incorporated at a concentration of 1 mol percent. Excitation and emission wavelengths λₑₓ=465 nm and λₑₘ=517 nm respectively. Error bars represent standard deviation, n=3.

FIG. 3 illustrates the effect of replacing PEG-CeramideC20 with PEG-Diacylglycerols on in vitro transfection potency of SPLP. Neuro-2a cells were treated with SPLP containing plasmids encoding the luciferase gene, under the control of the cytomegalovirus (CMV) promoter. The cells were subsequently lysed and luciferase concentrations determined. Error bars represent standard deviation, n=3.

FIG. 4 illustrates the % pharmacokinetics of SPLP containing PEG-CerC_{20,} PEG-S-DMG, PEG-S-DPG or PEG-S-DSG. The percentage of injected dose remaining in plasma of male A/J mice following a single intravenous administration is displayed. SPLP were labeled with ³H-cholesteryl hexadecyl ether (1 μCi per mg of lipid). Error bars represent the standard error of the mean (S.E.M.), n=4.

FIG. 5 illustrates the biodistribution of SPLP formulations containing the different PEG-lipids (diamonds=PEG-S-DMG, squares=PEG-S-DPG, triangles=PEG-S-DSG, open circles=PEG-C₂₀Ceramide). Following single intravenous administration of ³H-CHE-labeled SPLP in Neuro-2A tumor-bearing male AJ Mice, measurements were taken in the tumor (5a), liver (5b), lung (5c) and spleen (5d). Error bars represent the S.E.M., n=4. Tumors were 420 mg, ±30 mg (S.E.M.) at time of harvest.

FIG. 6 illustrates time course experiment showing luciferase gene expression in the tumor of male A/J mice following a single intravenous administration of SPLP containing PEG-Diacylglycerols. Injected dose was 200 μl total volume, containing 2 mg total lipid and 100 μg total DNA. Error bars represent the S.E.M., n=4. Tumors were 158 mg, ±60 mg (S.E.M.) at time of harvest.

FIG. 7 illustrates the biodistribution of luciferase gene expression in Neuro-2a tumor-bearing male A/J mice. Timepoint was 48 hrs after a single intravenous administration of SPLP containing PEG-CeramideC₂₀ or PEG-S-DAGs. Error bars represent the S.E.M., n=4. Tumors were 158 mg, ±60 mg (S.E.M.) at time of harvest. It should be noted that the y-axis is a log scale, unlike previous figures.

FIG. 8 Biodistribution of luciferase expression, represented as a function of DNA accumulation in Neuro-2a tumor-bearing male A/J mice. Timepoint was 48 hrs after a single intravenous administration of SPLP containing PEG-CeramideC₂₀ or PEG-S-DAGs. The considerable impact of tissue type on gene expression can be seen. Tumors were 158 mg, ±60 mg (S.E.M.) at time of harvest.

FIG. 9 illustrates data showing luciferase gene expression in tumors following IV administration of SPLP comprising PEG-DAA conjugates, PEG-DAG conjugates, and PEG-ceramide conjugates.

FIG. 10 illustrates data showing in vivo transfection by SPLP comprising PEG-DAA conjugates, PEG-DAG conjugates, PEG-ceramide conjugates, and PEG-DSPE conjugates.

FIG. 11 illustrates data showing luciferase gene expression in tumors 48 hours after intravenous administration of SPLP comprising PEG-DAA conjugates and PEG-DAG conjugates.

FIG. 12 illustrates data showing luciferase gene expression in liver, lung, spleen, heart, and tumor following intravenous administration of SPLP comprising PEG-DAA conjugates and PEG-DAG conjugates.

FIG. 13 illustrates data showing luciferase gene expression in tumors 48 hours after intravenous administration of SPLP or pSPLP comprising PEG-DAA conjugates and PEG-DAG conjugates.

FIG. 14 illustrates data showing in vivo transfection by SPLP comprising PEG-DAA conjugates and PEG-DAG conjugates.

FIG. 15 illustrates in vivo data demonstrating silencing of luciferase expression in Neuro-2a tumor bearing male A/J mice treated with SPLPs comprising a PEG-DAA conjugate and containing a plasmid encoding luciferase under the control of the CMV promoter and SNALPs comprising a PEG-DAA conjugate and containing anti-luciferase siRNA.

FIG. 16 illustrates in vivo data demonstrating silencing of luciferase expression in Neuro-2a tumor bearing male A/J mice treated with SPLPs comprising a PEG-DAA conjugate and containing a plasmid encoding luciferase under the control of the CMV promoter and SNALPs comprising a PEG-DAA conjugate and containing anti-luciferase siRNA.

FIG. 17 illustrates in vivo data demonstrating silencing of luciferase expression in Neuro-2a tumor bearing male A/J mice treated with SPLPs comprising a PEG-DAA conjugate and containing a plasmid encoding luciferase under the control of the CMV promoter and SNALPs comprising a PEG-DAA conjugate and containing anti-luciferase siRNA.

FIG. 18 illustrates in vivo data demonstrating silencing of luciferase expression in Neuro-2a tumor bearing male A/J mice treated with SPLPs comprising a PEG-DAA conjugate and containing a plasmid encoding luciferase under the control of the CMV promoter and SNALPs comprising a PEG-DAA conjugate and containing anti-luciferase siRNA.

FIG. 19 illustrates in vivo data demonstrating silencing of luciferase expression in Neuro-2a tumor bearing male A/J mice treated with SPLPs comprising a PEG-DAA conjugate and containing a plasmid encoding luciferase under the control of the CMV promoter and SNALPs comprising a PEG-DAA conjugate and containing anti-luciferase siRNA.

FIG. 20 illustrates data demonstrating uptake of SPLP comprising PEG-C-DMA conjugates by cells.

FIG. 21 illustrates data demonstrating the biodistribution of SPLP and SNALP comprising PEG-C-DMA or PEG-C-DSA in Neuro-2a tumor bearing male A/J mice 24 hours after administration of the SPLP or SNALP.

FIG. 22 illustrates data demonstrating the blood clearance of SPLP comprising PEG-C-DMA male A/J mice up to 24 hours after administration of the SPLP.

FIG. 23 illustrates data demonstrating the biodistribution of SPLP and SNALP comprising PEG-C-DMA in Neuro-2a tumor bearing male A/J mice 48 hours after administration of the SPLP or SNALP.

FIG. 24 illustrates data demonstrating the blood clearance of SPLP and SNALP comprising PEG-C-DMA or PEG-C-DSA in male A/J mice up to 24 hours after administration of the SPLP and SNALP.

FIG. 25 illustrates data demonstrating in vivo transfection by SPLP and pSPLP comprising PEG-DAA conjugates and PEG-DAG conjugates and encapsulating a plasmid encoding luciferase.

FIG. 26 illustrates data demonstrating in vivo transfection by SPLP comprising PEG-C-DMA conjugates and encapsulating a plasmid encoding luciferase.

FIG. 27 illustrates data demonstrating in vivo transfection by SPLP comprising PEG-C-DMA conjugates and encapsulating a plasmid encoding luciferase.

FIG. 28 illustrates data demonstrating silencing of luciferase expression in Neuro-2a cells contacted with SNALPs comprising a PEG-C-DMA conjugate and containing anti-luciferase siRNA.

FIG. 29 illustrates in vivo data demonstrating silencing of luciferase expression in metastatic Neuro-2a tumors in male A/J mice expressing luciferase and treated SNALPs comprising a PEG-C-DMA conjugate and encapsulating anti-luciferase siRNA.

FIG. 30A illustrates that SNALP encapsulating siRNA exhibit extended blood circulating times that are regulated by the PEG-lipid. Male A/J mice bearing subcutaneous Neuro2a tumors on the hind flank were treated with a single intravenous injection of radio-labeled SNALP (100 μg siRNA) containing either PEG-c-DSA or PEG-c-DMA (C18 or C14 alkyl chain length respectively). Whole blood samples were monitored for the non-exchangeable lipid marker 3H-cholestryl hexadecyl ether. Error bars represent standard errors of the mean (n=5). 50% of injected dose remains in the blood after 16 h and 3 h for SNALP containing PEG-c-DSA or PEG-c-DMA, respectively.

FIG. 30B illustrates that SNALP can be programmed to target specific disease sites including the liver and distal tumour. Biodistribution of radio-labeled SNALP was assessed after 24 h in tumour bearing mice described in FIG. 30A. PEG-c-DMA SNALP show preferential accumulation in the liver (35%) compared to PEG-c-DSA SNALP (13%). In contrast, PEG-c-DSA SNALP demonstrate enhanced targeting to the tumour site.

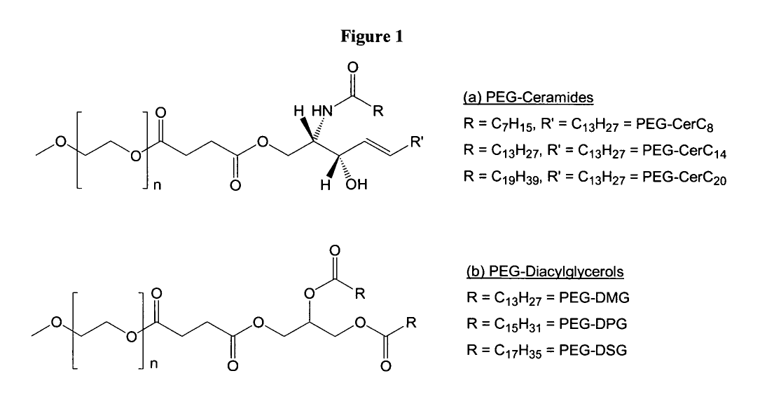

### DETAILED DESCRIPTION OF THE INVENTION AND PREFERRED EMBODIMENTS

I. Introduction

It has now been discovered that by controlling the length of the alkyl or acyl chains of the lipid portion of the PEG-lipid conjugate of a liposomal, SNALP or SPLP drug delivery system, one can control the circulation lifetime of the drug delivery system and, in turn, the biodistribution of the drug delivery vehicle. More particularly, by controlling the length of the alkyl or acyl chains of the lipid portion of the PEG-lipid conjugate, one can preferentially target the liposomal, SNALP or SPLP drug delivery system to a tumor or other target tissue of interest (e.g., the liver, lung, etc.). Thus, by controlling the length of the alkyl or acyl chains of the lipid of the PEG-lipid conjugate, one can modulate the time that the PEG-lipid conjugate remains associated with the bilayer and, in turn, the biodistribution of the liposomal, SNALP or SPLP drug delivery vehicle.

The present invention provides methods of introducing a nucleic acid into various tissues and cell types including, e.g., tumors, liver, lung, and spleen, by contacting the tissues or cells with a nucleic acid-lipid particle comprising a cationic lipid, a noncationic lipid, a PEG-lipid conjugate, and a nucleic acid.

In a preferred embodiment, the invention provides methods and compositions for preferential delivery of siRNA to a tumor site or other target tissue of interest. Again, longer chain PEG-lipid conjugates (e.g., C16, C18 or C20) result in the preferential delivery of the siRNA to a tumor site or the lung. Conversely, shorter chain PEG-lipid conjugates (e.g., C8, C12 or C14) result in the preferential delivery of the siRNA to the liver or spleen.

II. Definitions

The term “lipid” refers to a group of organic compounds that include, but are not limited to, esters of fatty acids and are characterized by being insoluble in water, but soluble in many organic solvents. They are usually divided into at least three classes: (1) “simple lipids” which include fats and oils as well as waxes; (2) “compound lipids” which include phospholipids and glycolipids; (3) “derived lipids” such as steroids.

“Lipid vesicle” refers to any lipid composition that can be used to deliver a compound including, but not limited to, liposomes, wherein an aqueous volume is encapsulated by an amphipathic lipid bilayer; or wherein the lipids coat an interior comprising a large molecular component, such as a plasmid comprising an interfering RNA sequence, with a reduced aqueous interior; or lipid aggregates or micelles, wherein the encapsulated component is contained within a relatively disordered lipid mixture.

As used herein, “lipid encapsulated” can refer to a lipid formulation that provides a compound with full encapsulation, partial encapsulation, or both. In a preferred embodiment, the nucleic acid is fully encapsulated in the lipid formulation (e.g., to form an SPLP, pSPLP, SNALP, or other nucleic-acid lipid particle). Nucleic-acid lipid particles and their method of preparation are disclosed in U.S. Pat. No. 5,976,567, U.S. Pat. No. 5,981,501 and WO 96/40964.

As used herein, the term “SNALP” refers to a stable nucleic acid lipid particle, including SPLP. A SNALP represents a vesicle of lipids coating a reduced aqueous interior comprising a nucleic acid (e.g., ssDNA, dsDNA, ssRNA, dsRNA, siRNA, or a plasmid, including plasmids from which an interfering RNA is transcribed). As used herein, the term “SPLP” refers to a nucleic acid lipid particle comprising a nucleic acid (e.g., a plasmid) encapsulated within a lipid vesicle. SNALPs and SPLPs typically contain a cationic lipid, a noncationic lipid, and a lipid that prevents aggregation of the particle (e.g., a PEG-lipid conjugate). SNALPs and SPLPs have systemic application as they exhibit extended circulation lifetimes following intravenous (i.v.) injection, accumulate at distal sites (e.g., sites physically separated from the administration site and can mediate expression of the transfected gene at these distal sites. SPLPs include “pSPLP” which comprise an encapsulated condensing agent-nucleic acid complex as set forth in WO 00/03683.

The term “vesicle-forming lipid” is intended to include any amphipathic lipid having a hydrophobic moiety and a polar head group, and which by itself can form spontaneously into bilayer vesicles in water, as exemplified by most phospholipids.

The term “vesicle-adopting lipid” is intended to include any amphipathic lipid that is stably incorporated into lipid bilayers in combination with other amphipathic lipids, with its hydrophobic moiety in contact with the interior, hydrophobic region of the bilayer membrane, and its polar head group moiety oriented toward the exterior, polar surface of the membrane. Vesicle-adopting lipids include lipids that on their own tend to adopt a nonlamellar phase, yet which are capable of assuming a bilayer structure in the presence of a bilayer-stabilizing component. A typical example is DOPE (dioleoylphosphatidylethanolamine). Bilayer stabilizing components include, but are not limited to, conjugated lipids that inhibit aggregation of the SNALPs, polyamide oligomers (e.g., ATTA-lipid derivatives), peptides, proteins, detergents, lipid-derivatives, PEG-lipid derivatives such as PEG coupled to dialkyloxypropyls, PEG coupled to diacylglycerols, PEG coupled to phosphatidyl-ethanolamines, and PEG conjugated to ceramides (see, e.g., U.S. Pat. No. 5,885,613). PEG can be conjugated directly to the lipid or may be linked to the lipid via a linker moiety. Any linker moiety suitable for coupling the PEG to a lipid can be used including, e.g., non-ester containing linker moieties and ester-containing linker moieties.

The term “amphipathic lipid” refers, in part, to any suitable material wherein the hydrophobic portion of the lipid material orients into a hydrophobic phase, while the hydrophilic portion orients toward the aqueous phase. Amphipathic lipids are usually the major component of a lipid vesicle. Hydrophilic characteristics derive from the presence of polar or charged groups such as carbohydrates, phosphate, carboxylic, sulfato, amino, sulfhydryl, nitro, hydroxy and other like groups. Hydrophobicity can be conferred by the inclusion of apolar groups that include, but are not limited to, long chain saturated and unsaturated aliphatic hydrocarbon groups and such groups substituted by one or more aromatic, cycloaliphatic or heterocyclic group(s). Examples of amphipathic compounds include, but are not limited to, phospholipids, aminolipids and sphingolipids. Representative examples of phospholipids include, but are not limited to, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine or dilinoleoylphosphatidylcholine. Other compounds lacking in phosphorus, such as sphingolipid, glycosphingolipid families, diacylglycerols and beta.-acyloxyacids, are also within the group designated as amphipathic lipids. Additionally, the amphipathic lipid described above can be mixed with other lipids including triglycerides and sterols.

The term “neutral lipid” refers to any of a number of lipid species that exist either in an uncharged or neutral zwitterionic form at a selected pH. At physiological pH, such lipids include, for example, diacylphosphatidylcholine, diacylphosphatidylethanolamine, ceramide, sphingomyelin, cephalin, cholesterol, cerebrosides and diacylglycerols.

The term “noncationic lipid” refers to any neutral lipid as described above as well as anionic lipids.

The term “anionic lipid” refers to any lipid that is negatively charged at physiological pH. These lipids include, but are not limited to, phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids.

The term “cationic lipid” refers to any of a number of lipid species that carry a net positive charge at a selected pH, such as physiological pH (e.g., pH of about 7.0). As used herein, physiological pH refers to the pH of a biological fluid such as blood or lymph as well as the pH of a cellular compartment such as an endosome, an acidic endosome, or a lysosome). Such lipids include, but are not limited to, N,N-dioleyl-N,N-dimethylammonium chloride (“DODAC”); N-(2,3-dioleyloxy)propyl)- N,N, N-trimethylammonium chloride (“DOTMA”); N,N-distearyl-N,N-dimethylammonium bromide (“DDAB”); N-(2,3-dioleoyloxy)propyl)-N,N,N-trimethylammonium chloride (“DOTAP”); 3 -(N-(N′,N′-dimethylaminoethane)-carbamoyl)cholesterol (“DC-Chol”); N-(1,2-dimyristyloxyprop-3-yl)-N,N-dimethyl-N-hydroxyethyl ammonium bromide (“DMRIE”); 1,2-Dilinoleyloxy-N,N-dimethylaminopropane (DLinDMA); and 1,2-Dilinolenyloxy-N,N-dimethylaminopropane (DLenDMA). The following lipids are cationic and have a positive charge at below physiological pH: DODAP, DODMA, DMDMA and the like.

The term “hydrophobic lipid” refers to compounds having apolar groups that include, but are not limited to, long chain saturated and unsaturated aliphatic hydrocarbon groups and such groups optionally substituted by one or more aromatic, cycloaliphatic or heterocyclic group(s). Suitable examples include, but are not limited to, diacylglycerol, dialkylglycerol, N-N-dialkylamino, 1,2-diacyloxy-3-aminopropane and 1,2-dialkyl-3-aminopropane.

The term “fusogenic” refers to the ability of a liposome, an SPLP, a SNALP or other drug delivery system to fuse with membranes of a cell. The membranes can be either the plasma membrane or membranes surrounding organelles, e.g., endosome, nucleus, etc.

The term “diacylglycerol” refers to a compound having 2-fatty acyl chains, R¹ and R², both of which have independently between 2 and 30 carbons bonded to the 1- and 2-position of glycerol by ester linkages. The acyl groups can be saturated or have varying degrees of unsaturation. Diacylglycerols have the following general formula:

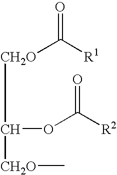

The term “dialkyloxypropyl” refers to a compound having 2-alkyl chains, R¹ and R², both of which have independently between 2 and 30 carbons. The alkyl groups can be saturated or have varying degrees of unsaturation. Dialkyloxypropyls have the following general formula:

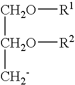

The term “PEG” refers to a polyethylene glycol, a linear, water-soluble polymer of ethylene PEG repeating units with two terminal hydroxyl groups. PEGs are classified by their molecular weights; for example, PEG 2000 has an average molecular weight of about 2,000 daltons, and PEG 5000 has an average molecular weight of about 5,000 daltons. PEGs are commercially available from Sigma Chemical Co. and other companies and include, for example, the following: monomethoxypolyethylene glycol (MePEG-OH), monomethoxypolyethylene glycol-succinate (MePEG-S), monomethoxypolyethylene glycol-succinimidyl succinate (MePEG-S-NHS), monomethoxypolyethylene glycol-amine (MePEG-NH₂), monomethoxypolyethylene glycol-tresylate (MePEG-TRES), and monomethoxypolyethylene glycol-imidazolyl-carbonyl (MePEG-IM). In addition, the example provide a protocol for synthesizing monomethoxypolyethyleneglycol-acetic acid (MePEG-CH₂COOH), which is particularly useful for preparing the PEG-DAA conjugates of the present invention.

In a preferred embodiment, the PEG is a polyethylene glycol with an average molecular weight of about 550 to about 10,000 daltons and is optionally substituted by alkyl, alkoxy, acyl or aryl. In a preferred embodiment, the PEG is substituted with methyl at the terminal hydroxyl position. In another preferred embodiment, the PEG has an average molecular weight of about 750 to about 5,000 daltons, more preferably, of about 1,000 to about 5,000 daltons, more preferably about 1,500 to about 3,000 daltons and, even more preferably, of about 2,000 daltons or of about 750 daltons. The PEG can be optionally substituted with alkyl, alkoxy, acyl or aryl. In a preferred embodiment, the terminal hydroxyl group is substituted with a methoxy or methyl group.

As used herein, a PEG-DAA conjugate refers to a polyethylene glycol conjugated to a dialkyloxypropyl. The PEG may be directly conjugated to the DAA or may be conjugated to the DAA via a linker moiety. Suitable linker moieties include nonester-containing linker moieties and ester containing linker moieties.

As used herein, the term “non-ester containing linker moiety” refers to a linker moiety that does not contain a carboxylic ester bond (—OC(O)—). Suitable non-ester containing linker moieties include, but are not limited to, amido (—C(O)NH—), amino (—NR—), carbonyl (—C(O)—), carbamate (—NHC(O)O—), urea (—NHC(O)NH—), disulphide (—S—S—), ether (—O—), succinyl (—(O)CCH₂CH₂C(O)—), succinamidyl (—NHC(O)CH₂CH₂C(O)NH—), ether, disulphide, etc. as well as combinations thereof (such as a linker containing both a carbamate linker moiety and an amido linker moiety). In a preferred embodiment, a carbamate linker is used to couple the PEG to the lipid.

In other embodiments, an ester containing linker moiety is used to couple the PEG to the lipid. Suitable ester containing linker moieties include, e.g., carbonate (—OC(O)O—), succinoyl, phosphate esters (—O—(O)POH—O—), sulfonate esters, and combinations thereof.

The term “ATTA” or “polyamide” refers to, but is not limited to, compounds disclosed in U.S. Pat. Nos. 6,320,017 and 6,586,559. These compounds include a compound having the formula

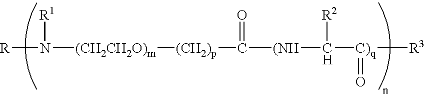

wherein: R is a member selected from the group consisting of hydrogen, alkyl and acyl; R¹ is a member selected from the group consisting of hydrogen and alkyl; or optionally, R and R¹ and the nitrogen to which they are bound form an azido moiety; R² is a member of the group selected from hydrogen, optionally substituted alkyl, optionally substituted aryl and a side chain of an amino acid; R³ is a member selected from the group consisting of hydrogen, halogen, hydroxy, alkoxy, mercapto, hydrazino, amino and NR⁴R⁵, wherein R⁴ and R⁵ are independently hydrogen or alkyl; n is 4 to 80; m is 2 to 6; p is 1 to 4; and q is 0 or 1. It will be apparent to those of skill in the art that other polyamides can be used in the compounds of the present invention.

The term “nucleic acid” or “polynucleotide” refers to a polymer containing at least two deoxyribonucleotides or ribonucleotides in either single- or double-stranded form. Nucleic acids include nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). Unless specifically limited, the terms encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzeret al., Nucleic Acid Res., 19:5081 (1991); Ohtsuka et al., J. Biol. Chem., 260:2605-2608 (1985); and Cassol et al. (1992); Rossolini et al., Mol. Cell. Probes, 8:91-98 (1994)). “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides. DNA may be in the form of antisense, plasmid DNA, parts of a plasmid DNA, pre-condensed DNA, product of a polymerase chain reaction (PCR), vectors (P1, PAC, BAC, YAC, artificial chromosomes), expression cassettes, chimeric sequences, chromosomal DNA, or derivatives of these groups. The term nucleic acid is used interchangeably with gene, cDNA, mRNA encoded by a gene, and an interfering RNA molecule.

The term “interfering RNA” or “RNAi” or “interfering RNA sequence” refers to double-stranded RNA (i.e., duplex RNA) that is capable of reducing or inhibiting expression of a target gene (i.e., by mediating the degradation of mRNAs which are complementary to the sequence of the interfering RNA) when the interfering RNA is in the same cell as the target gene. Interfering RNA thus refers to the double stranded RNA formed by two complementary strands or by a single, self-complementary strand. Interfering RNA typically has substantial or complete identity to the target gene. The sequence of the interfering RNA can correspond to the full length target gene, or a subsequence thereof. Interfering RNA includes small-interfering RNA” or “siRNA,” i.e., interfering RNA of about 15-60, 15-50, 15-50, or 15-40 (duplex) nucleotides in length, more typically about, 15-30, 15-25 or 19-25 (duplex) nucleotides in length, and is preferably about 20-24 or about 21-22 or 21-23 (duplex) nucleotides in length (e.g., each complementary sequence of the double stranded siRNA is 15-60, 15-50, 15-50, 15-40, 15-30, 15-25 or 19-25 nucleotides in length, preferably about 20-24 or about 21-22 or 21-23 nucleotides in length, and the double stranded siRNA is about 15-60, 15-50, 15-50, 15-40, 15-30, 15-25 or 19-25 preferably about 20-24 or about 21-22 or 21-23 base pairs in length). siRNA duplexes may comprise 3′ overhangs of about 1 to about 4 nucleotides, preferably of about 2 to about 3 nucleotides and 5′ phosphate termini. The siRNA can be chemically synthesized or maybe encoded by a plasmid (e.g., transcribed as sequences that automatically fold into duplexes with hairpin loops). siRNA can also be generated by cleavage of longer dsRNA (e.g., dsRNA greater than about 25 nucleotides in length) with the E coli RNase III or Dicer. These enzymes process the dsRNA into biologically active siRNA (see, e.g., Yang et al., PNAS USA, 99:9942-7 (2002); Calegari et al., PNAS USA, 99:14236 (2002); Byrom et al., Ambion TechNotes, 10(1):4-6 (2003); Kawasaki et al., Nucleic Acids Res., 31:981-7 (2003); Knight and Bass, Science, 293:2269-71 (2001); and Robertson et al., J. Biol. Chem., 243:82 (1968)). Preferably, dsRNA are at least 50 nucleotides to about 100, 200, 300, 400 or 500 nucleotides in length. A dsRNA may be as long as 1000, 1500, 2000, 5000 nucleotides in length, or longer. The dsRNA can encode for an entire gene transcript or a partial gene transcript.

The term “gene” refers to a nucleic acid (e.g., DNA or RNA) sequence that comprises partial length or entire length coding sequences necessary for the production of a polypeptide or a polypeptide precursor (e.g., polypeptides or polypeptide precursors from hepatitis virus A, B, C, D, E, or G; or herpes simplex virus).

“Gene product,” as used herein, refers to a product of a gene such as an RNA transcript, including, e.g., mRNA.

The phrase “inhibiting expression of a target gene” refers to the ability of a siRNA of the invention to initiate gene silencing of the target gene. To examine the extent of gene silencing, samples or assays of the organism of interest or cells in culture expressing a particular construct are compared to control samples lacking expression of the construct. Control samples (lacking construct expression) are assigned a relative value of 100%. Inhibition of expression of a target gene is achieved when the test value relative to the control is about 90%, preferably 50%, more preferably 25-0%. Suitable assays include, e.g., examination of protein or mRNA levels using techniques known to those of skill in the art such as dot blots, northern blots, in situ hybridization, ELISA, immunoprecipitation, enzyme function, as well as phenotypic assays known to those of skill in the art.

“Nucleic acid” refers to deoxyribonucleotides or ribonucleotides and polymers thereof in single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

By “silencing” or “downregulation” of a gene or nucleic acid is intended to mean a detectable decrease of translation of a target nucleic acid sequence, i.e., the sequence targeted by the siRNA, or a decrease in the amount or activity of the target sequence or protein, in comparison to the level that is detected in the absence of the siRNA sequence. A detectable decrease can be as small as about 5% or 10%, or as great as about 80%, 90% or 100%. More typically, a detectable decrease is about 20%, 30%, 40%, 50%, 60%, or 70%.

A “therapeutically effective amount” or an “effective amount” of a siRNA is an amount sufficient to produce the desired effect, e.g., a decrease in the expression of a target sequence in comparison to the normal expression level detected in the absence of the siRNA.

As used herein, the term “aqueous solution” refers to a composition comprising in whole, or in part, water.

As used herein, the term “organic lipid solution” refers to a composition comprising in whole, or in part, an organic solvent having a lipid.

“Distal site,” as used herein, refers to a physically separated site, which is not limited to an adjacent capillary bed, but includes sites broadly distributed throughout an organism. In some embodiments, distal site refers to a site physically separated from a disease site (e.g., the site of a tumor, the site of inflammation, or the site of an infection).

“Serum-stable” in relation to nucleic acid-lipid particles means that the particle is not significantly degraded after exposure to a serum or nuclease assay that would significantly degrade the free nucleic acid, e.g., DNA. Suitable assays include, for example, a standard serum assay or a DNAse assay such as those described in the Examples below.

“Systemic delivery,” as used herein, refers to delivery that leads to a broad biodistribution of a compound within an organism. Some techniques of administration can lead to the systemic delivery of certain compounds, but not others. Systemic delivery means that a useful, preferably therapeutic, amount of a compound is exposed to most parts of the body. To obtain broad biodistribution generally requires a blood lifetime such that the compound is not rapidly degraded or cleared (such as by first pass organs (liver, lung, etc.) or by rapid, nonspecific cell binding) before reaching a disease site distal to the site of administration. Systemic delivery of nucleic acid-lipid particules can be by any means known in the art including, for example, intravenous, subcutaneous, intraperitoneal, In a preferred embodiment, systemic delivery of nucleic acid-lipid particles is by intravenous delivery.

“Local delivery,” as used herein, refers to delivery of a compound directly to a target site within an organism. For example, a compound can be locally delivered by direct injection into a disease site such as a tumor or other target site such as a site of inflammation or a target organ such as the liver, heart, pancreas, kidney, and the like.

III. Lipid Based Carrier Systems Containing PEGLipid Conjugates

In one embodiment, the present invention provides stabilized nucleic acid-lipid particles (e.g., SPLPs and SNALPs) and other lipid-based carrier systems containing polyethyleneglycol (PEG)-lipid conjugates, e.g., PEG-dialkyloxypropyl (DAA) conjugates, PEG-diacylglycerol (DAG) conjugates, etc. The nucleic acid-lipid particles of the present invention typically comprise a nucleic acid, a cationic lipid, a noncationic lipid and a PEG-lipid conjugate.

The cationic lipid typically comprises from about 2% to about 60%, from about 5% to about 50%, from about 10% to about 45%, from about 20% to about 40%, or from about 30% to about 40% of the total lipid present in said particle. The noncationic lipid typically comprises from about 5% to about 90%, from about 10% to about 85%, from about 20% to about 80%, from about 30% to about 70%, from about 40% to about 60% or about 48% of the total lipid present in said particle. The PEG-lipid conjugate typically comprises from about 0.5% to about 20%, from about 1.5% to about 18%, from about 4% to about 15%, from about 5% to about 12%, or about 2% of the total lipid present in said particle. The lipid-based carrier systems (e.g., nucleic acid-lipid particles) of the present invention may further comprise cholesterol. If present, the cholesterol typically comprises from about 0% to about 10%, about 2% to about 10%, about 10% to about 60%, from about 12% to about 58%, from about 20% to about 55%, or about 48% of the total lipid present in said particle. It will be readily apparent to one of skill in the art that the proportions of the components of the lipid-based carrier systems (e.g., nucleic acid-lipid particles) may be varied. For example for systemic delivery, the cationic lipid may comprise from about 5% to about 15% of the total lipid present in said particle and for local or regional delivery, the cationic lipid may comprise from about 30% to about 50%, or about 40% of the total lipid present in said particle.

Depending on the intended use of the lipid-based carrier systems (e.g., nucleic acid-lipid particles), the proportions of the components are varied and the delivery efficiency of a particular formulation can be measured using an endosomal release parameter (ERP) assay. For example, for systemic delivery, the cationic lipid may comprise from about 5% to about 15% of the total lipid present in said particle and for local or regional delivery, the cationic lipid comprises from about 40% to about 50% of the total lipid present in said particle.

The nucleic acid-lipid particles of the present invention typically have a mean diameter of less than about 150 nm and are substantially nontoxic. In addition, the nucleic acids when present in the nucleic acid-lipid particles of the present invention are resistant to aqueous solution to degradation with a nuclease. Nucleic acid-lipid particles (e.g., SPLPs and SNALPs) and their method of preparation are disclosed in U.S. Pat. No. 5,976,567, U.S. Pat. No. 5,981,501 and WO 96/40964.

A. Cationic Lipids

Various suitable cationic lipids may be used in the lipid-based carrier systems (e.g., nucleic acid-lipid particles) described herein, either alone or in combination with one or more other cationic lipid species or neutral lipid species.

Cationic lipids which are useful in the present invention can be any of a number of lipid species which carry a net positive charge at physiological pH, for example: DLinDMA, DLenDMA, DODAC, DOTMA, DDAB, DOTAP, DOSPA, DOGS, DC-Chol and DMRIE, or combinations thereof. A number of these lipids and related analogs, which are also useful in the present invention, have been described in U.S. Pat. Nos. 5,208,036, 5,264,618, 5,279,833, 5,283,185, 5,753,613 and 5,785,992. Additionally, a number of commercial preparations of cationic lipids are available and can be used in the present invention. These include, for example, LIPOFECTIN® (commercially available cationic liposomes comprising DOTMA and DOPE, from GIBCO/BRL, Grand Island, New York, USA); LIPOFECTAMINE® (commercially available cationic liposomes comprising DOSPA and DOPE, from GIBCO/BRL); and TRANSFECTAM® (commercially available cationic liposomes comprising DOGS from Promega Corp., Madison, Wis., USA). In addition, cationic lipids of Formula II and Formula III can be used in the present invention. Cationic lipids of Formula II and III have the following structures:

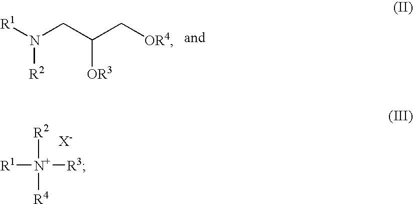

wherein R¹ and R² are independently selected and are H or C₁-C₃ alkyls. R³ and R⁴ are independently selected and are alkyl groups having from about 10 to about 20 carbon atoms; at least one of R³ and R⁴ comprises at least two sites of unsaturation. In one embodiment, R³ and R⁴ are both the same, i.e., R³ and R⁴ are both linoleyl (C18), etc. In another embodiment, R³ and R⁴ are different, i.e., R³ is myristyl (C14) and R⁴ is linoleyl (C18). In a preferred embodiment, the cationic lipids of the present invention are symmetrical, i.e., R³ and R⁴ are both the same. In another preferred embodiment, both R³ and R⁴ comprise at least two sites of unsaturation. In some embodiments, R³ and R⁴ are independently selected from dodecadienyl, tetradecadienyl, hexadecadienyl, linoleyl, and icosadienyl. In a preferred embodiment, R³ and R⁴ are both linoleyl. In some embodiments, R³ and R⁴comprise at least three sites of unsaturation and are independently selected from, e.g., dodecatrienyl, tetradectrienyl, hexadecatrienyl, linolenyl, and icosatrienyl.

The cationic lipids of Formula II and Formula III described herein typically carry a net positive charge at a selected pH, such as physiological pH. It has been surprisingly found that cationic lipids comprising alkyl chains with multiple sites of unsaturation, e.g., at least two or three sites of unsaturation, are particularly useful for forming lipid-nucleic acid particles with increased membrane fluidity. A number of cationic lipids and related analogs, which are also useful in the present invention, have been described in copending U.S. Ser. No. 08/316,399; U.S. Pat. Nos. 5,208,036, 5,264,618, 5,279,833 and 5,283,185, and WO 96/10390.

Additional suitable cationic lipids include, e.g., dioctadecyldimethylammonium (“DODMA”), Distearyldimethylammonium (“DSDMA”), N,N-dioleyl-N,N-dimethylammonium chloride (“DODAC”); N-(2,3-dioleyloxy)propyl)-N,N,N-trimethylammonium chloride (“DOTMA”); N,N-distearyl-N,N-dimethylammonium bromide (“DDAB”); N-(2,3-dioleoyloxy)propyl)-N,N,N-trimethylammonium chloride (“DOTAP”); 3-(N-(N′,N′-dimethylaminoethane)-carbamoyl)cholesterol (“DC-Chol”) and N-(1,2-dimyristyloxyprop-3-yl)-N,N-dimethyl-N-hydroxyethyl ammonium bromide (“DMRIE”). A number of these lipids and related analogs, which are also useful in the present invention, have been described in U.S. Pat. Nos. 5,208,036, 5,264,618, 5,279,833, 5,283,185, 5,753,613 and 5,785,992.

B. Noncationic Lipids

The noncationic lipid component of the lipid-based carrier systems (e.g., nucleic acid-lipid particles such as SPLPs and SNALPs) described herein can be any of a variety of neutral uncharged, zwitterionic or anionic lipids capable of producing a stable complex. They are preferably neutral, although they can alternatively be positively or negatively charged. Examples of noncationic lipids useful in the present invention include: phospholipid-related materials, such as lecithin, phosphatidylethanolamine, lysolecithin, lysophosphatidylethanolamine, phosphatidylserine, phosphatidylinositol, sphingomyelin, cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl- phosphatidylethanolamine (POPE) and dioleoyl- phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), and 1,2-dielaidoyl-sn-glycero-3-phophoethanolamine (transDOPE). Noncationic lipids or sterols such as cholesterol may be present. Additional nonphosphorous containing lipids are, e.g., stearylamine, dodecylamine, hexadecylamine, acetyl palmitate, glycerolricinoleate, hexadecyl stereate, isopropyl myristate, amphoteric acrylic polymers, triethanolamine-lauryl sulfate, alkyl-aryl sulfate polyethyloxylated fatty acid amides, dioctadecyldimethyl ammonium bromide and the like, diacylphosphatidylcholine, diacylphosphatidylethanolamine, ceramide, sphingomyelin, cephalin, and cerebrosides. Other lipids such as lysophosphatidylcholine and lysophosphatidylethanolamine may be present. Noncationic lipids also include polyethylene glycol-based polymers such as PEG 2000, PEG 5000 and polyethylene glycol conjugated to phospholipids or to ceramides (referred to as PEG-Cer), as described in U.S. Pat. No. 5,820,873.

In preferred embodiments, the noncationic lipids are diacylphosphatidylcholine (e.g., distearoylphosphatidylcholine, dioleoylphosphatidylcholine, dipalmitoylphosphatidylcholine and dilinoleoylphosphatidylcholine), diacylphosphatidylethanolamine (e.g., dioleoylphosphatidylethanolamine and palmitoyloleoylphosphatidylethanolamine), ceramide or sphingomyelin. The acyl groups in these lipids are preferably acyl groups derived from fatty acids having C₁₀-C₂₄ carbon chains. More preferably the acyl groups are lauroyl, myristoyl, palmitoyl, stearoyl or oleoyl. In particularly preferred embodiments, the noncationic lipid will include one or more of cholesterol, 1,2-sn-dioleoylphosphatidylethanolamine, or egg sphingomyelin (ESM).

C. PEGLipid Conjugates

In one embodiment, the lipid-based carrier systems (e.g., nucleic acid-lipid particles) further comprise a PEG-lipids, such as PEG coupled to dialkyloxypropyls (PEG-DAA), PEG coupled to diacylglycerol (PEG-DAG), PEG coupled to phosphatidylethanolamine (PE) (PEG-PE), or PEG conjugated to ceramides, or a mixture thereof (see, e.g., U.S. Pat. No. 5,885,613). In one embodiment, the bilayer stabilizing component is a PEG-lipid, or an ATTA-lipid.

The PEG-lipid conjugate typically comprises from about 0.5% to about 20%, from about 1.5% to about 18%, from about 4% to about 15%, from about 5% to about 12%, or about 2% of the total lipid present in said particle. One of ordinary skill in the art will appreciate that the concentration of the PEG-lipid conjugate can be varied depending on the bilayer stabilizing component employed and the rate at which the liposome is to become fusogenic.

By controlling the length of the alkyl or acyl chains of the lipid of the PEG-lipid conjugate, one can determine the time that the PEG-lipid conjugate remains associated with the bilayer and, in turn, the biodistribution of the liposomal, SNALP or SPLP drug delivery vehicle. Again, longer chain PEG-lipid conjugates (e.g., C16, C18 or C20) allow for the liposomal, SNALP or SPLP drug delivery vehicle to be preferentially delivered to a tumor site or the lung. Conversely, shorter chain PEG-lipid conjugates (e.g., C8, C12 or C14) allow for the liposomal, SNALP or SPLP drug delivery vehicle to be preferentially delivered to the liver.

1 . Diacylglycerol-polyethyleneglycol conjugates

In one embodiment, the bilayer stabilizing component comprises a diacylglycerol-polyethyleneglycol conjugate, i.e., a DAG-PEG conjugate or a PEG-DAG conjugate. In a preferred embodiment, the DAG-PEG conjugate is a dilaurylglycerol (C₁₂)-PEG conjugate, dimyristylglycerol (C₁₄)-PEG conjugate (DMG), a dipalmitoylglycerol (C₁₆)-PEG conjugate or a distearylglycerol (C₁₈)-PEG conjugate (DSG). Those of skill in the art will readily appreciate that other diacylglycerols can be used in the DAG-PEG conjugates of the present invention. Suitable DAG-PEG conjugates for use in the present invention, and methods of making and using them, are disclosed in U.S. application Ser. No. 10/136,707 published as U.S. Patent Application No. 2003/0077829, and PCT Patent Application No. CA 02/00669, each of which is incorporated herein in its entirety by reference.

2. Dialkyloxypropyl conjugates

In another embodiment, the bilayer stabilizing component comprises a dialkyloxypropyl conjugate, i.e., a PEG-DAA conjugate. Such PEG-DAA conjugates have increased stability over commonly used PEG-lipid conjugates (such as PEG-PE conjugates). In one preferred embodiment, the PEG-DAA conjugates of Formula I have the following structure: 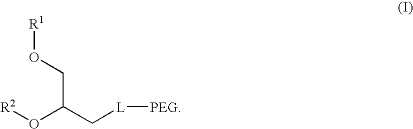
In Formula I, above, R¹ and R² are independently selected and are alkyl groups having from about 10 to about 20 carbon atoms. The alkyl groups can be saturated or unsaturated. Suitable alkyl groups include, but are not limited to, lauryl (C12), myristyl (C14), palmityl (C16), stearyl (C18) and icosyl (C20). In one embodiment, R¹ and R² are both the same, i.e., R¹ and R² are both myristyl (C14) or both stearyl (C18), etc. In another embodiment, R¹ and R² are different, i.e., R¹ is myristyl (C¹⁴) and R² is stearyl (C¹⁸). In a preferred embodiment, the PEG-DAA conjugates of the present invention are symmetrical, i.e., R¹ and R² are both the same.

In Formula I, above, PEG is a polyethylene glycol, a linear, water-soluble polymer of ethylene PEG repeating units with two terminal hydroxyl groups. PEGs are classified by their molecular weights; for example, PEG 2000 has an average molecular weight of about 2,000 daltons, and PEG 5000 has an average molecular weight of about 5,000 daltons. PEGs are commercially available from Sigma Chemical Co. and other companies and include, for example, the following: monomethoxypolyethylene glycol (MePEG-OH), monomethoxypolyethylene glycol-succinate (MePEG-S), monomethoxypolyethylene glycol-succinimidyl succinate (MePEG-S-NHS), monomethoxypolyethylene glycol-amine (MePEG-NH₂), monomethoxypolyethylene glycol-tresylate (MePEG-TRES), and monomethoxypolyethylene glycol-imidazolyl-carbonyl (MePEG-IM). In addition, the example provide a protocol for synthesizing monomethoxypolyethyleneglycol-acetic acid (MePEG-CH₂COOH), which is particularly useful for preparing the PEG-DAA conjugates of the present invention.

In a preferred embodiment, the PEG is a polyethylene glycol with an average molecular weight of about 550 to about 10,000 daltons and is optionally substituted by alkyl, alkoxy, acyl or aryl. In a preferred embodiment, the PEG is substituted with methyl at the terminal hydroxyl position. In another preferred embodiment, the PEG has an average molecular weight of about 750 to about 5,000 daltons, more preferably, of about 1,000 to about 5,000 daltons, more preferably about 1,500 to about 3,000 daltons and, even more preferably, of about 2,000 daltons or of about 750 daltons.

In Formula I, above, “L” is a non-ester containing linker moiety or an ester containing linker moiety. In a preferred embodiment, L is a non-ester containing linker moiety, i.e., a linker moiety that does not contain a carboxylic ester bond (—OC(O)—). Suitable non-ester containing linkers include, but are not limited to, an amido linker moiety, an amino linker moiety, a carbonyl linker moiety, a carbamate linker moiety, a urea linker moiety, an ether linker moiety, a disulphide linker moiety, a succinamidyl linker moiety, a succinyl linker moiety, and combinations thereof. In a preferred embodiment, the non-ester containing linker moiety is a carbamate linker moiety (i.e., a PEG-C-DAA conjugate). In another preferred embodiment, the non-ester containing linker moiety is an amido linker moiety (i.e., a PEG-A-DAA conjugate). In a preferred embodiment, the non-ester containing linker moiety is a succinamidyl linker moiety (i.e., a PEG-S-DAA conjugate).

In other embodiments, L is an ester containing linker moiety. Suitable ester containing linker moieties include, e.g., carbonate (—OC(O)O—), succinoyl, phosphate esters (—O—(O)POH—O—), sulfonate esters, and combinations thereof.

The PEG-DAA conjugates of the present invention are synthesized using standard techniques and reagents known to those of skill in the art. It will be recognized that the PEG-DAA conjugates of the present invention will contain various amide, amine, ether, thio, carbamate and urea linkages. Those of skill in the art will recognize that methods and reagents for forming these bonds are well known and readily available. See, e.g., March, ADVANCED ORGANIC CHEMISTRY (Wiley 1992), Larock, COMPREHENSIVE ORGANIC TRANSFORMATIONS (VCH 1989); and Furniss, VOGEL'S TEXTBOOK OF PRACTICAL ORGANIC CHEMISTRY 5th ed. (Longman 1989). It will also be appreciated that any functional groups present may require protection and deprotection at different points in the synthesis of the PEG-DAA conjugates of the present invention. Those of skill in the art will recognize that such techniques are well known. See, e.g., Green and Wuts, PROTECTIVE GROUPS IN ORGANIC SYNTHESIS (Wiley 1991).

A general sequence of reactions for forming the PEG-DAA conjugates of the present invention is set forth in Example Section below. The examples provide synthesis schemes for preparing PEG-A-DMA, PEG-C-DMA and PEG-S-DMA conjugates of the present invention. Using similar protocols, one of skill in the art can readily generate the other PEG-DAA conjugates of the present invention.

In addition to the foregoing, it will be readily apparent to those of skill in the art that other hydrophilic polymers can be used in place of PEG. Examples of suitable polymers that can be used in place of PEG include, but are not limited to, polyvinylpyrrolidone, polymethyloxazoline, polyethyloxazoline, polyhydroxypropyl methacrylamide, polymethacrylamide and polydimethylacrylamide, polylactic acid, polyglycolic acid, and derivatized celluloses, such as hydroxymethylcellulose or hydroxyethylcellulose.

3. Other PEGLipid Conjugates

Phosphatidylethanolamines having a variety of acyl chain groups of varying chain lengths and degrees of saturation can be conjugated to polyethyleneglycol to form the bilayer stabilizing component. Such phosphatidylethanolamines are commercially available, or can be isolated or synthesized using conventional techniques known to those of skilled in the art. Phosphatidylethanolamines containing saturated or unsaturated fatty acids with carbon chain lengths in the range of C₁₀ to C₂₀ are preferred. Phosphatidylethanolamines with mono- or diunsaturated fatty acids and mixtures of saturated and unsaturated fatty acids can also be used. Suitable phosphatidylethanolamines include, but are not limited to, the following: dimyristoylphosphatidylethanolamine (DMPE), dipalmitoylphosphatidylethanolamine (DPPE), dioleoylphosphatidylethanolamine (DOPE) and distearoylphosphatidylethanolamine (DSPE).

As with the phosphatidylethanolamines, ceramides having a variety of acyl chain groups of varying chain lengths and degrees of saturation can be coupled to polyethyleneglycol to form the bilayer stabilizing component. It will be apparent to those of skill in the art that in contrast to the phosphatidylethanolamines, ceramides have only one acyl group which can be readily varied in terms of its chain length and degree of saturation. Ceramides suitable for use in accordance with the present invention are commercially available. In addition, ceramides can be isolated, for example, from egg or brain using well-known isolation techniques or, alternatively, they can be synthesized using the methods and techniques disclosed in U.S. Pat. No. 5,820,873. Using the synthetic routes set forth in the foregoing application, ceramides having saturated or unsaturated fatty acids with carbon chain lengths in the range of C₂ to C₃₁ can be prepared.

D. Products of Interest

In addition to the above components, the lipid-based carrier systems (e.g., nucleic acid-lipid particles such as SPLPs and SNALPs) of the present invention comprise a nucleic acid (e.g., single stranded or double stranded DNA, single stranded or double stranded RNA, RNAi, siRNA, and the like). Suitable nucleic acids include, but are not limited to, plasmids, antisense oligonucleotides, ribozymes as well as other poly- and oligonucleotides. In preferred embodiments, the nucleic acid encodes a product, e.g., a therapeutic product, of interest.

The product of interest can be useful for commercial purposes, including for therapeutic purposes as a pharmaceutical or diagnostic. Examples of therapeutic products include a protein, a nucleic acid, an antisense nucleic acid, ribozymes, tRNA, snRNA, siRNA, an antigen, Factor VIII, and Apoptin (Zhuang et al. (1995) Cancer Res. 55(3): 486-489). Suitable classes of gene products include, but are not limited to, cytotoxic/suicide genes, immunomodulators, cell receptor ligands, tumor suppressors, and anti-angiogenic genes. The particular gene selected will depend on the intended purpose or treatment. Examples of such genes of interest are described below and throughout the specification.

1. siRNA

In some embodiments, the nucleic acid component of the nucleic acid-lipid particles (e.g., SNALPs and SPLPs) typically comprise an interfering RNA (i.e., siRNA), which can be provided in several forms including, e.g.,as one or more isolated small-interfering RNA (siRNA) duplexes, longer double-stranded RNA (dsRNA) or as siRNA or dsRNA transcribed from a transcriptional cassette in a DNA plasmid.

An RNA population can be used to provide long precursor RNAs, or long precursor RNAs that have substantial or complete identity to a selected target sequence can be used to make the siRNA. The RNAs can be isolated from cells or tissue, synthesized, and/or cloned according to methods well known to those of skill in the art. The RNA can be a mixed population (obtained from cells or tissue, transcribed from cDNA, subtracted, selected etc.), or can represent a single target sequence. RNA can be naturally occurring, e.g., isolated from tissue or cell samples, synthesized in vitro, e.g., using T7 or SP6 polymerase and PCR products or a cloned cDNA; or chemically synthesized.

To form a long dsRNA, for synthetic RNAs, the complement is also transcribed in vitro and hybridized to form a ds RNA. If a naturally occuring RNA population is used, the RNA complements are also provided (e.g., to form dsRNA for digestion by E. coli RNAse III or Dicer), e.g., by transcribing cDNAs corresponding to the RNA population, or by using RNA polymerases. The precursor RNAs are then hybridized to form double stranded RNAs for digestion. The dsRNAs can be directlu emcapsulated in the SNALPs or can be digested in vitro prior to encapsulation.

Alternatively, one or more DNA plasmids encoding one or more siRNA templates are encapsulated in a nucleic acid-lipid particle. siRNA can be transcribed as sequences that automatically fold into duplexes with hairpin loops from DNA templates in plasmids having RNA polymerase III transcriptional units, for example, based on the naturally occurring transcription units for small nuclear RNA U6 or human RNase P RNA H1 (see, Brummelkamp et al., Science, 296:550 (2002); Donze et al., Nucleic Acids Res., 30:e46 (2002); Paddison et al., Genes Dev., 16:948 (2002); Yu et al., Proc. Natl. Acad. Sci., 99:6047 (2002); Lee et al., Nat. Biotech., 20:500 (2002); Miyagishi et al., Nat. Biotech., 20:497 (2002); Paul et al., Nat. Biotech., 20:505 (2002); and Sui et al., Proc. Natl. Acad. Sci., 99:5515 (2002)). Typically, a transcriptional unit or cassette will contain an RNA transcript promoter sequence, such as an H1-RNA or a U6 promoter, operably linked to a template for transcription of a desired siRNA sequence and a termination sequence, comprised of 2-3 uridine residues and a polythymidine (T5) sequence (polyadenylation signal) (Brummelkamp, Science, supra). The selected promoter can provide for constitutive or inducible transcription. Compositions and methods for DNA-directed transcription of RNA interference molecules is described in detail in U.S. Pat. No. 6,573,099. Preferably, the synthesized or transcribed siRNA have 3′ overhangs of about 1-4 nucleotides, preferably of about 2-3 nucleotides and 5′ phosphate termini (Elbashir et al., Genes Dev., 15:188 (2001); Nykanen et al., Cell, 107:309 (2001)). The transcriptional unit is incorporated into a plasmid or DNA vector from which the interfering RNA is transcribed. Plasmids suitable for in vivo delivery of genetic material for therapeutic purposes are described in detail in U.S. Pat. Nos. 5,962,428 and 5,910,488. The selected plasmid can provide for transient or stable delivery of a target cell. It will be apparent to those of skill in the art that plasmids originally designed to express desired gene sequences can be modified to contain a transcriptional unit cassette for transcription of siRNA.

Methods for isolating RNA, synthesizing RNA, hybridizing nucleic acids, making and screening cDNA libraries, and performing PCR are well known in the art (see, e.g., Gubler & Hoffman, Gene, 25:263-269 (1983); Sambrook et al., supra; Ausubel et al., supra), as are PCR methods (see U.S. Pat. Nos. 4,683,195 and 4,683,202; PCR Protocols: A Guide to Methods and Applications (Innis et al., eds, 1990)). Expression libraries are also well known to those of skill in the art. Additional basic texts disclosing the general methods of use in this invention include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)).

A suitable plasmid is engineered to contain, in expressible form, a template sequence that encodes a partial length sequence or an entire length sequence of a gene product of interest. Template sequences can also be used for providing isolated or synthesized siRNA and dsRNA. Generally, it is desired to downregulate or silence the transcription and translation of a gene product of interest. Suitable classes of gene products include, but are not limited to, genes associated with tumorigenesis and cell transformation, angiogenic genes, immunomodulator genes, such as those associated with inflammatory and autoimmune responses, ligand receptor genes, genes associated with neurodegenerative disorders, and genes associated with viral infection and survival.

Examples of gene sequences associated with tumorigenesis and cell transformation include translocation sequences such as MLL fusion genes, BCR-ABL (Wilda et al., Oncogene, 21:5716 (2002); Scherr et al., Blood, 101:1566), TEL-AML1, EWS-FLI1, TLS-FUS, PAX3-FKHR, BCL-2, AML1-ETO and AML1-MTG8 (Heidenreich et al., Blood, 101:3157 (2003)); overexpressed sequences such as multidrug resistance genes (Nieth et al., FEBS Lett., 545:144 (2003); Wu et al, Cancer Res., 63:1515 (2003)), cyclins (Li et al., Cancer Res., 63:3593 (2003); Zou et al., Genes Dev., 16:2923 (2002)), beta-Catenin (Verma et al., Clin Cancer Res., 9:1291 (2003)), telomerase genes (Kosciolek et al., Mol. Cancer Ther., 2:209 (2003)), c-MYC, N-MYC, BCL-2, ERBB1 and ERBB2 (Nagy et al. Exp. Cell Res., 285:39 (2003)); and mutated sequences such as RAS (reviewed in Tuschl and Borkhardt, Mol. Interventions, 2:158 (2002)). Silencing of sequences that encode DNA repair enzymes find use in combination with the administration of chemotherapeutic agents (Collis et al., Cancer Res., 63:1550 (2003)). Genes encoding proteins associated with tumor migration are also target sequences of interest, for example, integrins, selectins and metalloproteinases. The foregoing examples are not exclusive. Any whole or partial gene sequence that facilitates or promotes tumorigenesis or cell transformation, tumor growth or tumor migration can be included as a template sequence

Angiogenic genes are able to promote the formation of new vessels. Of particular interest is Vascular Endothelial Growth Factor (VEGF) (Reich et al., Mol. Vis., 9:210 (2003)).

Immunomodulator genes are genes that modulate one or more immune responses. Examples of immunomodulator genes include cytokines such as growth factors (e.g., TGF-α., TGF-β, EGF, FGF, IGF, NGF, PDGF, CGF, GM-CSF, SCF, etc.), interleukins (e.g., IL-2, IL-4, IL-12 (Hill et al., J. Immunol., 171:691 (2003)), IL-1 5, IL-1 8, IL-20, etc. interferons (e.g., IFN-α, IFN-β, IFN-γ, etc.) and TNF. Fas and Fas Ligand genes are also immunomodulator target sequences of interest (Song et al., Nat. Med., 9:347 (2003)). Genes encoding secondary signaling molecules in hematopoietic and lymphoid cells are also included in the present invention, for example, Tec family kinases, such as Bruton's tyrosine kinase (Btk) (Heinonen et al., FEBS Lett., 527:274 (2002)).

Cell receptor ligands include ligands that are able to bind to cell surface receptors (e.g., insulin receptor, EPO receptor, G-protein coupled receptors, receptors with tyrosine kinase activity, cytokine receptors, growth factor receptors, etc.), to modulate (e.g,. inhibit, activate, etc.) the physiological pathway that the receptor is involved in (e.g., glucose level modulation, blood cell development, mitogenesis, etc.). Examples of cell receptor ligands include cytokines, growth factors, interleukins, interferons, erythropoietin (EPO), insulin, glucagon, G-protein coupled receptor ligands, etc.). Templates coding for an expansion of trinucleotide repeats (e.g., CAG repeats), find use in silencing pathogenic sequences in neurodegenerative disorders caused by the expansion of trinucleotide repeats, such as spinobulbular muscular atrophy and Huntington's Disease (Caplen et al., Hum. Mol. Genet., 11:175 (2002)).

Genes associated with viral infection and survival include those expressed by a virus in order to bind, enter and replicate in a cell. Of particular interest are viral sequences associated with chronic viral diseases. Viral sequences of particular interest include sequences of Human Immunodeficiency Virus (HIV) (Banerjea et al., Mol. Ther., 8:62 (2003); Song et al., J. Virol., 77:7174 (2003); Stephenson, JAMA, 289:1494 (2003); Qin et al., Proc. Natl. Acad. Sci., 100:183 (2003)), Hepatitis viruses (Hamasaki et al., FEBS Lett., 543:51 (2003); Yokota et al., EMBO Rep., 4:602 (2003); Schlomai et al., Hepatology, 37:764 (2003); Wilson et al., Proc. Natl. Acad. Sci., 100:2783 (2003); Kapadia et al., Proc. Natl. Acad. Sci., 100:2014 (2003)), Herpes viruses (Jia et al., J. Virol., 77:3301 (2003)), and Human Papilloma Viruses (HPV) (Hall et al., J. Virol., 77:6066 (2003); Jiang et al., Oncogene, 21:6041 (2002)).

2. Additional Therapeutic Products

As explained above, in some embodiments of the present invention, the SPLPs and SNALPs encapsulate a nucleic acid encoding a therapeutic product such as, for example, tumor suppressor genes, immunomodulator genes, cell receptor ligand genes, anti-antigogenic genes, and cytotoxic/suicide genes.

a) Tumor Suppressors

Tumor suppressor genes are genes that are able to inhibit the growth of a cell, particularly tumor cells. Thus, delivery of these genes to tumor cells is useful in the treatment of cancers. Tumor suppressor genes include, but are not limited to, p53 (Lamb et al., Mol. Cell. Biol., 6:1379-1385 (1986), Ewen et al., Science, 255:85-87 (1992), Ewen et al., Cell, 66:1155-1164 (1991), and Hu et al., EMBO J., 9:1147-1155 (1990)), RB1 (Toguchida et al., Genomics, 17:535-543 (1993)), WT1 (Hastie, Curr. Opin. Genet. Dev., 3:408-413 (1993)), NF1 (Trofatter et al., Cell, 72:791-800 (1993), Cawthon et al., Cell, 62:193-201 (1990)), VHL (Latif et al., Science, 260:1317-1320 (1993)), APC (Gorden et al., Cell, 66:589-600 (1991)), DAP kinase (see, e.g., Diess et al., Genes Dev., 9:15-30 (1995)), p16 (see, e.g., Marx, Science, 264(5167):1846 (1994)), ARF (see, e.g., Quelle et al., Cell, 83(6): 993-1000 (1995)), Neurofibromin (see, e.g., Huynh et al., Neurosci. Lett., 143(1-2):233-236 (1992)), and PTEN (see, e.g., Li et al., Science, 275(5308):1943-1947 (1997)).

b) Immunomodulator Genes:

Immunomodulator genes are genes that modulate one or more immune responses. Examples of immunomodulator genes include cytokines such as growth factors (e.g., TGF-α., TGF-β, EGF, FGF, IGF, NGF, PDGF, CGF, GM-CSF, G-CSF, SCF, etc.), interleukins (e.g., IL-2, IL-3, IL-4, IL-6, IL-7, IL-10, IL-12, IL-15, IL-20, etc.), interferons (e.g., IFN-α, IFN-β, IFN-γ, etc.), TNF (e.g., TNF-α), and Flt3-Ligand.

c) Cell Receptor ligands

Cell receptor ligands include ligands that are able to bind to cell surface receptors (e.g., insulin receptor, EPO receptor, G-protein coupled receptors, receptors with tyrosine kinase activity, cytokine receptors, growth factor receptors, etc.), to modulate (e.g,. inhibit, activate, etc.) the physiological pathway that the receptor is involved in (e.g., glucose level modulation, blood cell development, mitogenesis, etc.). Examples of cell receptor ligands include, but are not limited to, cytokines, growth factors, interleukins, interferons, erythropoietin (EPO), insulin, single-chain insulin (Lee et al. (2000) Nature 408:483-488), glucagon, G-protein coupled receptor ligands, etc.). These cell surface ligands can be useful in the treatment of patients suffering from a disease. For example, a single-chain insulin when expressed under the control of the glucose-responsive hepatocyte-specific L-type pyruvate kinase (LPK) promoter was able to cause the remission of diabetes in streptocozin-induced diabetic rats and autoimmune diabetic mice without side effects (Lee et al., Nature, 408:483-488 (2000)). This single-chain insulin was created by replacing the 35 amino acid resides of the C-peptide of insulin with a short turn-forming heptapeptide (Gly-Gly-Gly-Pro-Gly-Lys-Arg).

d) Anti-Angiogenic Genes

Anti-angiogenic genes are able to inhibit neovascularization. These genes are particularly useful for treating those cancers in which angiogenesis plays a role in the pathological development of the disease. Examples of anti-angiogenic genes include, but are not limited to, endostatin (see, e.g., U.S. Pat. No. 6,174,861), angiostatin (see, e.g., U.S. Pat. No. 5,639,725), and VEGF-R2 (see, e.g., Decaussin et al., J. Pathol., 188(4): 369-737 (1999)).

e) Cytotoxic/Suicide Genes

Cytotoxic/suicide genes are those genes that are capable of directly or indirectly killing cells, causing apoptosis, or arresting cells in the cell cycle. Such genes include, but are not limited to, genes for immunotoxins, a herpes simplex virus thymidine kinase (HSV-TK), a cytosine deaminase, a xanthine-guaninephosphoribosyl transferase, a p53, a purine nucleoside phosphorylase, a carboxylesterase, a deoxycytidine kinase, a nitroreductase, a thymidine phosphorylase, and a cytochrome P450 2B 1.

In a gene therapy technique known as gene-delivered enzyme prodrug therapy (“GDEPT”) or, alternatively, the “suicide gene/prodrug” system, agents such as acyclovir and ganciclovir (for thymidine kinase), cyclophosphoamide (for cytochrome P450 2B 1), 5-fluorocytosine (for cytosine deaminase), are typically administered systemically in conjunction (e.g., simultaneously or nonsimultaneously, e.g., sequentially) with a expression cassette encoding a suicide gene compositions of the present invention to achieve the desired cytotoxic or cytostatic effect (see, e.g., Moolten, Cancer Res., 46:5276-5281 (1986)). For a review of the GDEPT system, see, Moolten, The Internet Book of Gene Therapy, Cancer Therapeutics, Chapter 11 (Sobol, R. E., Scanlon, N. J. (Eds) Appelton & Lange (1995)). In this method, a heterologous gene is delivered to a cell in an expression cassette containing a RNAP promoter, the heterologous gene encoding an enzyme that promotes the metabolism of a first compound to which the cell is less sensitive (i.e., the “prodrug”) into a second compound to which is cell is more sensitive. The prodrug is delivered to the cell either with the gene or after delivery of the gene. The enzyme will process the prodrug into the second compound and respond accordingly. A suitable system proposed by Moolten is the herpes simplex virus-thymidine kinase (HSV-TK) gene and the prodrug ganciclovir. This method has recently been employed using cationic lipid-nucleic aggregates for local delivery (i.e., direct intra-tumoral injection), or regional delivery (i.e., intra-peritoneal) of the TK gene to mouse tumors by Zerrouqui et al., Can. Gen. Therapy, 3(6):385-392 (1996); Sugaya et al., Hum. Gen. Ther., 7:223-230 (1996) and Aoki et al., Hum. Gen. Ther., 8:1105-1113 (1997). Human clinical trials using a GDEPT system employing viral vectors have been proposed (see, Hum. Gene Ther., 8:597-613 (1997), and Hum. Gene Ther., 7:255-267 (1996)) and are underway.

For use with the instant invention, the most preferred therapeutic products are those which are useful in gene-delivered enzyme prodrug therapy (“GDEPT”). Any suicide gene/prodrug combination can be used in accordance with the present invention. Several suicide gene/prodrug combinations suitable for use in the present invention are cited in Sikora, K. in OECD Documents, Gene Delivery Systems at pp. 59-71 (1996), include, but are not limited to, the following:

| Suicide Gene Product | Less Active ProDrug | Activated Drug |
|---|---|---|
| Herpes simplex virus | ganciclovir(GCV), | phosphorylated |
| type 1 thymidine | acyclovir, | dGTP analogs |
| kinase (HSV-TK) | bromovinyl- | |
| | deoxyuridine, or other | |
| | substrates | |
| Cytosine Deaminase | 5-fluorocytosine | 5-fluorouracil |
| (CD) | | |
| Xanthine-guanine- | 6-thioxanthine (6TX) | 6-thioguano- |
| phosphoribosyl | | sinemonophosphate |
| transferase (XGPRT) | | |
| Purine nucleoside | MeP-dr | 6-methylpurine |
| phosphorylase | | |
| Cytochrome P450 | cyclophosphamide | [cytotoxic |
| 2B1 | | metabolites] |
| Linamarase | amygdalin | cyanide |
| Nitroreductase | CB 1954 | nitrobenzamidine |
| Beta-lactamase | PD | PD mustard |
| Beta-glucuronidase | adria-glu | adriamycin |
| Carboxypeptidase | MTX-alanine | MTX |
| Glucose oxidase | glucose | peroxide |
| Penicillin amidase | adria-PA | adriamycin |
| Superoxide dismutase | XRT | DNA damaging agent |
| Ribonuclease | RNA | cleavage products |

Any prodrug can be used if it is metabolized by the heterologous gene product into a compound to which the cell is more sensitive. Preferably, cells are at least 10-fold more sensitive to the metabolite than the prodrug.

Modifications of the GDEPT system that may be useful with the invention include, for example, the use of a modified TK enzyme construct, wherein the TK gene has been mutated to cause more rapid conversion of prodrug to drug (see, for example, Black et al., Proc. Natl. Acad. Sci, U.S.A., 93:3525-3529 (1996)). Alternatively, the TK gene can be delivered in a bicistronic construct with another gene that enhances its effect. For example, to enhance the “bystander effect” also known as the “neighbor effect” (wherein cells in the vicinity of the transfected cell are also killed), the TK gene can be delivered with a gene for a gap junction protein, such as connexin 43. The connexin protein allows diffusion of toxic products of the TK enzyme from one cell into another. The TK/Connexin 43 construct has a CMV promoter operably linked to a TK gene by an internal ribosome entry sequence and a Connexin 43-encoding nucleic acid.

E. Other Components

Cationic polymer lipids (CPLs) can also be used in the nucleic acid-lipid particles (e.g., SNALPs or SPLPs) described herein. Suitable CPL typically have the following architectural features: (1) a lipid anchor, such as a hydrophobic lipid, for incorporating the CPLs into the lipid bilayer; (2) a hydrophilic spacer, such as a polyethylene glycol, for linking the lipid anchor to a cationic head group; and (3) a polycationic moiety, such as a naturally occurring amino acid, to produce a protonizable cationic head group. Suitable SNALPs and SNALP-CPLs for use in the present invention, and methods of making and using SNALPs and SNALP-CPLs, are disclosed, e.g., in U.S. application Ser. Nos. 09/553,639 and 09/839,707 (published as U.S.P.A. 2002/0072121) and PCT Patent Application No. CA 00/00451 (published as WO 00/62813), each of which is incorporated herein in its entirety by reference.

Briefly, the present invention provides a compound of Formula II:
A-W—Y I

wherein A, W and Y are as follows.

With reference to Formula II, “A” is a lipid moiety such as an amphipathic lipid, a neutral lipid or a hydrophobic lipid that acts as a lipid anchor. Suitable lipid examples include vesicle-forming lipids or vesicle adopting lipids and include, but are not limited to, diacylglycerolyls, dialkylglycerolyls, N-N-dialkylaminos, 1,2-diacyloxy-3-aminopropanes and 1,2-dialkyl-3-aminopropanes.

“W” is a polymer or an oligomer, such as a hydrophilic polymer or oligomer. Preferably, the hydrophilic polymer is a biocompatable polymer that is nonimmunogenic or possesses low inherent immunogenicity. Alternatively, the hydrophilic polymer can be weakly antigenic if used with appropriate adjuvants. Suitable nonimmunogenic polymers include, but are not limited to, PEG, polyamides, polylactic acid, polyglycolic acid, polylactic acid/polyglycolic acid copolymers and combinations thereof. In a preferred embodiment, the polymer has a molecular weight of about 250 to about 7000 daltons.

“Y” is a polycationic moiety. The term polycationic moiety refers to a compound, derivative, or functional group having a positive charge, preferably at least 2 positive charges at a selected pH, preferably physiological pH. Suitable polycationic moieties include basic amino acids and their derivatives such as arginine, asparagine, glutamine, lysine and histidine; spermine; spermidine; cationic dendrimers; polyamines; polyamine sugars; and amino polysaccharides. The polycationic moieties can be linear, such as linear tetralysine, branched or dendrimeric in structure. Polycationic moieties have between about 2 to about 15 positive charges, preferably between about 2 to about 12 positive charges, and more preferably between about 2 to about 8 positive charges at selected pH values. The selection of which polycationic moiety to employ may be determined by the type of liposome application which is desired.

The charges on the polycationic moieties can be either distributed around the entire liposome moiety, or alternatively, they can be a discrete concentration of charge density in one particular area of the liposome moiety e.g., a charge spike. If the charge density is distributed on the liposome, the charge density can be equally distributed or unequally distributed. All variations of charge distribution of the polycationic moiety are encompassed by the present invention.

The lipid “A,” and the nonimmunogenic polymer “W,” can be attached by various methods and preferably, by covalent attachment. Methods known to those of skill in the art can be used for the covalent attachment of “A” and “W.” Suitable linkages include, but are not limited to, amide, amine, carboxyl, carbonate, carbamate, ester and hydrazone linkages. It will be apparent to those skilled in the art that “A” and “W” must have complementary functional groups to effectuate the linkage. The reaction of these two groups, one on the lipid and the other on the polymer, will provide the desired linkage. For example, when the lipid is a diacylglycerol and the terminal hydroxyl is activated, for instance with NHS and DCC, to form an active ester, and is then reacted with a polymer which contains an amino group, such as with a polyamide (see, e.g., U.S. Pat. Nos. 6,320,017 and 6,586,559), an amide bond will form between the two groups.

In certain instances, the polycationic moiety can have a ligand attached, such as a targeting ligand or a chelating moiety for complexing calcium. Preferably, after the ligand is attached, the cationic moiety maintains a positive charge. In certain instances, the ligand that is attached has a positive charge. Suitable ligands include, but are not limited to, a compound or device with a reactive functional group and include lipids, amphipathic lipids, carrier compounds, bioaffinity compounds, biomaterials, biopolymers, biomedical devices, analytically detectable compounds, therapeutically active compounds, enzymes, peptides, proteins, antibodies, immune stimulators, radiolabels, fluorogens, biotin, drugs, haptens, DNA, RNA, polysaccharides, liposomes, virosomes, micelles, immunoglobulins, functional groups, other targeting moieties, or toxins.

F. Nucleic Acid-lipid Particle Preparation and Uses Thereof

The present invention provides methods for preparing serum-stable nucleic acid-lipid particles such that the nucleic acid (e.g., siRNA or plasmid encoding siRNA) is encapsulated in a lipid bilayer and is protected from degradation. The nucleic acid-lipid particles (e.g., SNALPs and SPLPs) made by the methods of this invention are typically about 50 to about 150 nm in diameter. They generally have a median diameter of less than about 150 nm, more typically a diameter of less than about 100 nm, with a majority of the particles having a median diameter of about 65 to 85 nm. Exemplary methods of making nucleic acid-lipid particles are disclosed in U.S. Pat. Nos. 5,705,385; 5,981,501; 5,976,567; 6,586,410; 6,534,484; U.S. patent application Ser. No. 09/553,639; U.S. Patent Publication Nos. 2002/0072121 and 2003/0077829); WO 96/40964; and WO 00/62813.

In one embodiment, the present invention provides nucleic acid-lipid particles produced via a process that includes providing an aqueous solution in a first reservoir, and providing an organic lipid solution in a second reservoir, and mixing the aqueous solution with the organic lipid solution such that the organic lipid solution mixes with the aqueous solution so as to substantially instantaneously produce a liposome encapsulating the nucleic acid. This process and the apparatus for carrying this process is described in detail in U.S. Patent Publication No. 2004/0142025.

The action of continuously introducing lipid and buffer solutions into a mixing environment, such as in a mixing chamber, causes a continuous dilution of the lipid solution with the buffer solution, thereby producing a liposome substantially instantaneously upon mixing. As used herein, the phrase “continuously diluting a lipid solution with a buffer solution” (and variations) generally means that the lipid solution is diluted sufficiently rapidly in an hydration process with sufficient force to effectuate vesicle generation. By mixing the aqueous solution with the organic lipid solution, the organic lipid solution undergoes a continuous stepwise dilution in the presence of the buffer (aqueous) solution to produce a liposome.

As mentioned above, the nucleic acid-lipid particles of the present invention, i.e., those nucleic acid-lipid particles containing PEG-lipid conjugates, can be made using any of a number of different methods known in the art including, e.g., a detergent dialysis method or by modification of a reverse-phase method which utilizes organic solvents to provide a single phase during mixing of the components. Without intending to be bound by any particular mechanism of formation, a plasmid or other nucleic acid (i.e., siRNA) is contacted with a detergent solution of cationic lipids to form a coated nucleic acid complex. These coated nucleic acids can aggregate and precipitate. However, the presence of a detergent reduces this aggregation and allows the coated nucleic acids to react with excess lipids (typically, noncationic lipids) to form particles in which the plasmid or other nucleic acid is encapsulated in a lipid bilayer. The methods described below for the formation of nucleic acid-lipid particles using organic solvents follow a similar scheme.

In one embodiment, the present invention provides lipid-nucleic acid particles produced via hydrophobic nucleic acid-lipid intermediate complexes. The complexes are preferably charge-neutralized. Manipulation of these complexes in either detergent-based or organic solvent-based systems can lead to particle formation in which the nucleic acid is protected.

The present invention provides a method of preparing serum-stable nucleic acid-lipid particles in which a nucleic acid is encapsulated in a lipid bilayer and is protected from degradation. Additionally, the particles formed in the present invention are preferably neutral or negatively-charged at physiological pH. For in vivo applications, neutral particles are advantageous, while for in vitro applications the particles are more preferably negatively charged. This provides the further advantage of reduced aggregation over the positively-charged liposome formulations in which a nucleic acid can be encapsulated in cationic lipids.

The particles made by the methods of this invention have a size of about 50 to about 150 nm, with a majority of the particles being about 65 to 85 nm. The particles can be formed by either a detergent dialysis method or by a modification of a reverse-phase method which utilizes organic solvents to provide a single phase during mixing of the components. Without intending to be bound by any particular mechanism of formation, a plasmid or other nucleic acid is contacted with a detergent solution of cationic lipids to form a coated plasmid complex. These coated plasmids can aggregate and precipitate. However, the presence of a detergent reduces this aggregation and allows the coated plasmids to react with excess lipids (typically, noncationic lipids) to form particles in which the plasmid or other nucleic acid is encapsulated in a lipid bilayer. The methods described below for the formation of plasmid-lipid particles using organic solvents follow a similar scheme.

In some embodiments, the particles are formed using detergent dialysis. Thus, the present invention provides a method for the preparation of serum-stable nucleic acid-lipid particles, comprising:

(a) combining a nucleic acid with cationic lipids in a detergent solution to form a coated plasmid-lipid complex;

(b) contacting noncationic lipids with the coated nucleic acid-lipid complex to form a detergent solution comprising a plasmid-lipid complex and noncationic lipids; and

(c) dialyzing the detergent solution of step (b) to provide a solution of serum-stable nucleic acid-lipid particles, wherein the nucleic acid is encapsulated in a lipid bilayer and the particles are serum-stable and have a size of from about 50 to about 150 nm.

An initial solution of coated nucleic acid-lipid complexes is formed by combining the plasmid with the cationic lipids in a detergent solution.

In these embodiments, the detergent solution is preferably an aqueous solution of a neutral detergent having a critical micelle concentration of 15-300 mM, more preferably 20-50 mM. Examples of suitable detergents include, for example, N,N′-((octanoylimino)-bis-(trimethylene))-bis-(D-gluconamide) (BIGCHAP); BRIJ 35; Deoxy-BIGCHAP; dodecylpoly(ethylene glycol) ether; Tween 20; Tween 40; Tween 60; Tween 80; Tween 85; Mega 8; Mega 9; Zwittergent® 3-08; Zwittergent® 3-10; Triton X-405; hexyl-, heptyl-, octyl- and nonyl-β-D-glucopyranoside; and heptylthioglucopyranoside; with octyl β-D-glucopyranoside and Tween-20 being the most preferred. The concentration of detergent in the detergent solution is typically about 100 mM to about 2 M, preferably from about 200 mM to about 1.5 M.

The cationic lipids and nucleic acids will typically be combined to produce a charge ratio (±) of about 1:1 to about 20: 1, preferably in a ratio of about 1:1 to about 12:1, and more preferably in a ratio of about 2:1 to about 6:1. Additionally, the overall concentration of plasmid in solution will typically be from about 25 μg/mL to about 1 mg/mL, preferably from about 25 μg/mL to about 500 μg/mL, and more preferably from about 100 μg/mL to about 250 μg/mL. The combination of nucleic acids and cationic lipids in detergent solution is kept, typically at room temperature, for a period of time which is sufficient for the coated complexes to form. Alternatively, the nucleic acids and cationic lipids can be combined in the detergent solution and warmed to temperatures of up to about 37° C. For nucleic acids which are particularly sensitive to temperature, the coated complexes can be formed at lower temperatures, typically down to about 4° C.

In a preferred embodiment, the nucleic acid to lipid ratios (mass/mass ratios) in a formed SPLP or SNALP will range from about 0.01 to about 0.2, from about 0.03 to about 0.01, or about 0.01 to about 0.08. The ratio of the starting materials also falls within this range. In another preferred embodiment, the SPLP or SNALP preparation uses about 400 μg nucleic acid per 10 mg total lipid or a nucleic acid to lipid ratio of about 0.01 to about 0.08 and, more preferably, about 0.04, which corresponds to 1.25 mg of total lipid per 50 μg of nucleic acid.

The detergent solution of the coated nucleic acid-lipid complexes is then contacted with noncationic lipids to provide a detergent solution of nucleic acid-lipid complexes and noncationic lipids. The noncationic lipids which are useful in this step include, diacylphosphatidylcholine, diacylphosphatidylethanolamine, ceramide, sphingomyelin, cephalin, cardiolipin, and cerebrosides. In preferred embodiments, the noncationic lipids are diacylphosphatidylcholine, diacylphosphatidylethanolamine, ceramide or sphingomyelin. The acyl groups in these lipids are preferably acyl groups derived from fatty acids having C₁₀-C₂₄ carbon chains. More preferably the acyl groups are lauroyl, myristoyl, palmitoyl, stearoyl or oleoyl. In particularly preferred embodiments, the noncationic lipid will be 1,2-sn-dioleoylphosphatidylethanolamine (DOPE), palmitoyl oleoyl phosphatidylcholine (POPC), egg phosphatidylcholine (EPC), distearoylphosphatidylcholine (DSPC), cholesterol, or a mixture thereof. In the most preferred embodiments, the nucleic acid-lipid particles will be fusogenic particles with enhanced properties in vivo and the noncationic lipid will be DSPC or DOPE. As explained above, the nucleic acid-lipid particles of the present invention will further comprise PEG-lipid conjugates. In addition, the nucleic acid-lipid particles of the present invention will further comprise cholesterol.

Following formation of the detergent solution of nucleic acid-lipid complexes and noncationic lipids, the detergent is removed, preferably by dialysis. The removal of the detergent results in the formation of a lipid-bilayer which surrounds the nucleic acid providing serum-stable nucleic acid-lipid particles which have a size of from about 50 nm to about 150 nm. The particles thus formed do not aggregate and are optionally sized to achieve a uniform particle size.

The serum-stable nucleic acid-lipid particles can be sized by any of the methods available for sizing liposomes. The sizing may be conducted in order to achieve a desired size range and relatively narrow distribution of particle sizes.

Several techniques are available for sizing the particles to a desired size. One sizing method, used for liposomes and equally applicable to the present particles is described in U.S. Pat. No. 4,737,323. Sonicating a particle suspension either by bath or probe sonication produces a progressive size reduction down to particles of less than about 50 nm in size. Homogenization is another method which relies on shearing energy to fragment larger particles into smaller ones. In a typical homogenization procedure, particles are recirculated through a standard emulsion homogenizer until selected particle sizes, typically between about 60 and 80 nm, are observed. In both methods, the particle size distribution can be monitored by conventional laser-beam particle size discrimination, or QELS.

Extrusion of the particles through a small-pore polycarbonate membrane or an asymmetric ceramic membrane is also an effective method for reducing particle sizes to a relatively well-defined size distribution. Typically, the suspension is cycled through the membrane one or more times until the desired particle size distribution is achieved. The particles may be extruded through successively smaller-pore membranes, to achieve a gradual reduction in size.

In another group of embodiments, the present invention provides a method for the preparation of serum-stable nucleic acid-lipid particles, comprising:

(a) preparing a mixture comprising cationic lipids and noncationic lipids in an organic solvent;

(b) contacting an aqueous solution of nucleic acid with said mixture in step (a) to provide a clear single phase; and

(c) removing said organic solvent to provide a suspension of nucleic acid-lipid particles, wherein said nucleic acid is encapsulated in a lipid bilayer, and said particles are stable in serum and have a size of from about 50 to about 150 nm.

The nucleic acids (e.g., plasmids), cationic lipids and noncationic lipids which are useful in this group of embodiments are as described for the detergent dialysis methods above.

The selection of an organic solvent will typically involve consideration of solvent polarity and the ease with which the solvent can be removed at the later stages of particle formation. The organic solvent, which is also used as a solubilizing agent, is in an amount sufficient to provide a clear single phase mixture of plasmid and lipids. Suitable solvents include, but are not limited to, chloroform, dichloromethane, diethylether, cyclohexane, cyclopentane, benzene, toluene, methanol, or other aliphatic alcohols such as propanol, isopropanol, butanol, tert-butanol, iso-butanol, pentanol and hexanol. Combinations of two or more solvents may also be used in the present invention.

Contacting the nucleic acid with the organic solution of cationic and noncationic lipids is accomplished by mixing together a first solution of nucleic acids, which is typically an aqueous solution, and a second organic solution of the lipids. One of skill in the art will understand that this mixing can take place by any number of methods, for example by mechanical means such as by using vortex mixers.

After the nucleic acid has been contacted with the organic solution of lipids, the organic solvent is removed, thus forming an aqueous suspension of serum-stable nucleic acid-lipid particles. The methods used to remove the organic solvent will typically involve evaporation at reduced pressures or blowing a stream of inert gas (e.g., nitrogen or argon) across the mixture.

The serum-stable nucleic acid-lipid particles thus formed will typically be sized from about 50 nm to 150 nm. To achieve further size reduction or homogeneity of size in the particles, sizing can be conducted as described above.

In other embodiments, the methods will further comprise adding nonlipid polycations which are useful to effect the transformation of cells using the present compositions. Examples of suitable nonlipid polycations include, but are limited to, hexadimethrine bromide (sold under the brandname POLYBRENE®, from Aldrich Chemical Co., Milwaukee, Wis., USA) or other salts of heaxadimethrine. Other suitable polycations include, for example, salts of poly-L-omithine, poly-L-arginine, poly-L-lysine, poly-D-lysine, polyallylamine and polyethyleneimine.

In some embodiments, the polycations can be used to condense nucleic acids prior to encapsulation of the nucleic acids in the SPLP or SNALP. For example, the polycation (e.g., polyethyleneimine) can be used as a condensing agent to form a PEI-condensed DNA complex as described in WO 00/03683.

In certain embodiments, the formation of the nucleic acid-lipid particles can be carried out either in a mono-phase system (e.g., a Bligh and Dyer monophase or similar mixture of aqueous and organic solvents) or in a two-phase system with suitable mixing.

When formation of the complexes is carried out in a mono-phase system, the cationic lipids and nucleic acids are each dissolved in a volume of the mono-phase mixture. Combination of the two solutions provides a single mixture in which the complexes form. Alternatively, the complexes can form in two-phase mixtures in which the cationic lipids bind to the nucleic acid (which is present in the aqueous phase), and “pull” it into the organic phase.

In another embodiment, the present invention provides a method for the preparation of nucleic acid-lipid particles, comprising:

(a) contacting nucleic acids with a solution comprising noncationic lipids and a detergent to form a nucleic acid-lipid mixture;

(b) contacting cationic lipids with the nucleic acid-lipid mixture to neutralize a portion of the negative charge of the nucleic acids and form a charge-neutralized mixture of nucleic acids and lipids; and

(c) removing the detergent from the charge-neutralized mixture to provide the lipid-nucleic acid particles in which the nucleic acids are protected from degradation.

In one group of embodiments, the solution of noncationic lipids and detergent is an aqueous solution. Contacting the nucleic acids with the solution of noncationic lipids and detergent is typically accomplished by mixing together a first solution of nucleic acids and a second solution of the lipids and detergent. One of skill in the art will understand that this mixing can take place by any number of methods, for example, by mechanical means such as by using vortex mixers. Preferably, the nucleic acid solution is also a detergent solution. The amount of noncationic lipid which is used in the present method is typically determined based on the amount of cationic lipid used, and is typically of from about 0.2 to 5 times the amount of cationic lipid, preferably from about 0.5 to about 2 times the amount of cationic lipid used.

The nucleic acid-lipid mixture thus formed is contacted with cationic lipids to neutralize a portion of the negative charge which is associated with the nucleic acids (or other polyanionic materials) present. The amount of cationic lipids used will typically be sufficient to neutralize at least 50% of the negative charge of the nucleic acid. Preferably, the negative charge will be at least 70% neutralized, more preferably at least 90% neutralized. Cationic lipids which are useful in the present invention, include, for example, DODAC, DOTMA, DDAB, DOTAP, DC-Chol and DMRIE. These lipids and related analogs have been described in copending U.S. Ser. No. 08/316,399; U.S. Pat. Nos. 5,208,036, 5,264,618, 5,279,833 and 5,283,185. Additionally, a number of commercial preparations of cationic lipids are available and can be used in the present invention. These include, for example, LIPOFECTIN® (commercially available cationic liposomes comprising DOTMA and DOPE, from GIBCO/BRL, Grand Island, N.Y., USA); LIPOFECTAMINE® (commercially available cationic liposomes comprising DOSPA and DOPE, from GIBCO/BRL); and TRANSFECTAM® (commercially available cationic lipids comprising DOGS in ethanol from Promega Corp., Madison, Wis., USA).

Contacting the cationic lipids with the nucleic acid-lipid mixture can be accomplished by any of a number of techniques, preferably by mixing together a solution of the cationic lipid and a solution containing the nucleic acid-lipid mixture. Upon mixing the two solutions (or contacting in any other manner), a portion of the negative charge associated with the nucleic acid is neutralized. Nevertheless, the nucleic acid remains in an uncondensed state and acquires hydrophilic characteristics.

After the cationic lipids have been contacted with the nucleic acid-lipid mixture, the detergent (or combination of detergent and organic solvent) is removed, thus forming the lipid-nucleic acid particles. The methods used to remove the detergent will typically involve dialysis. When organic solvents are present, removal is typically accomplished by evaporation at reduced pressures or by blowing a stream of inert gas (e.g., nitrogen or argon) across the mixture.

The particles thus formed will typically be sized from about 100 nm to several microns. To achieve further size reduction or homogeneity of size in the particles, the lipid-nucleic acid particles can be sonicated, filtered or subjected to other sizing techniques which are used in liposomal formulations and are known to those of skill in the art.

In other embodiments, the methods will further comprise adding nonlipid polycations which are useful to effect the lipofection of cells using the present compositions. Examples of suitable nonlipid polycations include, hexadimethrine bromide (sold under the brandname POLYBRENE®, from Aldrich Chemical Co., Milwaukee, Wis., USA) or other salts of hexadimethrine. Other suitable polycations include, for example, salts of poly-L-omithine, poly-L-arginine, poly-L-lysine, poly-D-lysine, polyallylamine and polyethyleneimine. Addition of these salts is preferably after the particles have been formed.

In another aspect, the present invention provides methods for the preparation of nucleic acid-lipid particles, comprising:

(a) contacting an amount of cationic lipids with nucleic acids in a solution; the solution comprising from about 15-35% water and about 65-85% organic solvent and the amount of cationic lipids being sufficient to produce a ±charge ratio of from about 0.85 to about 2.0, to provide a hydrophobic lipid-nucleic acid complex;

(b) contacting the hydrophobic, lipid-nucleic acid complex in solution with noncationic lipids, to provide a nucleic acid-lipid mixture; and

(c) removing the organic solvents from the lipid-nucleic acid mixture to provide lipid-nucleic acid particles in which the nucleic acids are protected from degradation.

The nucleic acids, noncationic lipids, cationic lipids and organic solvents which are useful in this aspect of the invention are the same as those described for the methods above which used detergents. In one group of embodiments, the solution of step (a) is a mono-phase. In another group of embodiments, the solution of step (a) is two-phase.

In preferred embodiments, the cationic lipids are DLinDMA, DLenDMA, DODAC, DDAB, DOTMA, DOSPA, DMRIE, DOGS or combinations thereof. In other preferred embodiments, the noncationic lipids are ESM, DOPE, DOPC, DSPC, polyethylene glycol-based polymers (e.g., PEG 2000, PEG 5000, PEG-modified diacylglycerols, or PEG-modified dialkyloxypropyls), distearoylphosphatidylcholine (DSPC), cholesterol, or combinations thereof. In still other preferred embodiments, the organic solvents are methanol, chloroform, methylene chloride, ethanol, diethyl ether or combinations thereof.

In a particularly preferred embodiment, the nucleic acid is a plasmid or siRNA; the cationic lipid is DLinDMA, DLenDMA, DODAC, DDAB, DOTMA, DOSPA, DMRIE, DOGS or combinations thereof; the noncationic lipid is ESM, DOPE, PEG-lipids (such as PEG-DAAs or PEG-DAGs), distearoylphosphatidylcholine (DSPC), cholesterol, or combinations thereof (e.g.,DSPC and PEG-DAAs); and the organic solvent is methanol, chloroform, methylene chloride, ethanol, diethyl ether or combinations thereof.

As above, contacting the nucleic acids with the cationic lipids is typically accomplished by mixing together a first solution of nucleic acids and a second solution of the lipids, preferably by mechanical means such as by using vortex mixers. The resulting mixture contains complexes as described above. These complexes are then converted to particles by the addition of noncationic lipids and the removal of the organic solvent. The addition of the noncationic lipids is typically accomplished by simply adding a solution of the noncationic lipids to the mixture containing the complexes. A reverse addition can also be used. Subsequent removal of organic solvents can be accomplished by methods known to those of skill in the art and also described above.

The amount of noncationic lipids which is used in this aspect of the invention is typically an amount of from about 0.2 to about 15 times the amount (on a mole basis) of cationic lipids which was used to provide the charge-neutralized lipid-nucleic acid complex. Preferably, the amount is from about 0.5 to about 9 times the amount of cationic lipids used.

In yet another aspect, the present invention provides lipid-nucleic acid particles which are prepared by the methods described above. In these embodiments, the lipid-nucleic acid particles are either net charge neutral or carry an overall charge which provides the particles with greater gene lipofection activity. Preferably, the nucleic acid component of the particles is a nucleic acid which encodes a desired protein or blocks the production of an undesired protein. In preferred embodiments, the nucleic acid is a plasmid, the noncationic lipid is egg sphingomyelin and the cationic lipid is DODAC. In particularly preferred embodiments, the nucleic acid is a plasmid, the noncationic lipid is a mixture of DSPC and cholesterol, and the cationic lipid is DLinDMA. In other particularly preferred embodiments, the noncationic lipid may further comprise cholesterol.

A variety of general methods for making nucleic acid-lipid particles such as, for example, SPLP-CPLs (CPL-containing SPLPs) or SNALP-CPL's (CPL-containing SNALPs) are discussed herein. Two general techniques include “post-insertion” technique, that is, insertion of a CPL into for example, a pre-formed SPLP or SNALP, and the “standard” technique, wherein the CPL is included in the lipid mixture during for example, the SPLP or SNALP formation steps. The post-insertion technique results in SPLPs having CPLs mainly in the external face of the SPLP or SNALP bilayer membrane, whereas standard techniques provide SPLPs or SNALPs having CPLs on both internal and external faces.

In particular, “post-insertion” involves forming SPLPs or SNALPs (by any method), and incubating the pre-formed SPLPs or SNALPs in the presence of CPL under appropriate conditions (preferably 2-3 hours at 60° C.). Between 60-80% of the CPL can be inserted into the external leaflet of the recipient vesicle, giving final concentrations up to about 5 to 10 mol % (relative to total lipid). The method is especially useful for vesicles made from phospholipids (which can contain cholesterol) and also for vesicles containing PEG-lipids (such as PEG-DAAs).

In an example of a “standard” technique, the CPL-SPLPs and CPL-SNALPs of the present invention can be formed by extrusion. In this embodiment, all of the lipids including the CPL, are co-dissolved in chloroform, which is then removed under nitrogen followed by high vacuum. The lipid mixture is hydrated in an appropriate buffer, and extruded through two polycarbonate filters with a pore size of 100 nm. The resulting SPLPs or SNALPs contain CPL on both of the internal and external faces. In yet another standard technique, the formation of CPL-SPLPs or CPL-SNALPs can be accomplished using a detergent dialysis or ethanol dialysis method, for example, as discussed in U.S. Pat. Nos. 5,976,567 and 5,981,501.

The nucleic acid-lipid particles of the present invention can be administered either alone or in mixture with a physiologically-acceptable carrier (such as physiological saline or phosphate buffer) selected in accordance with the route of administration and standard pharmaceutical practice. Generally, normal saline will be employed as the pharmaceutically acceptable carrier. Other suitable carriers include, e.g., water, buffered water, 0.4% saline, 0.3% glycine, and the like, including glycoproteins for enhanced stability, such as albumin, lipoprotein, globulin, etc.

The pharmaceutical carrier is generally added following particle formation. Thus, after the particle is formed, the particle can be diluted into pharmaceutically acceptable carriers such as normal saline.

The concentration of particles in the pharmaceutical formulations can vary widely, i.e., from less than about 0.05%, usually at or at least about 2-5% to as much as 10 to 30% by weight and will be selected primarily by fluid volumes, viscosities, etc., in accordance with the particular mode of administration selected. For example, the concentration may be increased to lower the fluid load associated with treatment. This may be particularly desirable in patients having atherosclerosis-associated congestive heart failure or severe hypertension. Alternatively, particles composed of irritating lipids may be diluted to low concentrations to lessen inflammation at the site of administration.

As described above, the nucleic acid-lipid particles of the present invention comprise PEG-lipid conjugates. It is often desirable to include other components that act in a manner similar to the PEG-lipid conjugates and that serve to prevent particle aggregation and to provide a means for increasing circulation lifetime and increasing the delivery of the nucleic acid-lipid particles to the target tissues. Such components include, but are not limited to, PEG-lipid conjugates, such as PEG-ceramides or PEG-phospholipids (such as PEG-PE), ganglioside G_{M1}-modified lipids or ATTA-lipids to the particles. Typically, the concentration of the component in the particle will be about 1-20 % and, more preferably from about 3-10 %.

The pharmaceutical compositions of the present invention may be sterilized by conventional, well known sterilization techniques. Aqueous solutions can be packaged for use or filtered under aseptic conditions and lyophilized, the lyophilized preparation being combined with a sterile aqueous solution prior to administration. The compositions can contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, and calcium chloride. Additionally, the particle suspension may include lipid-protective agents which protect lipids against free-radical and lipid-peroxidative damages on storage. Lipophilic free-radical quenchers, such as alphatocopherol and water-soluble iron-specific chelators, such as ferrioxamine, are suitable.

In another example of their use, lipid-nucleic acid particles can be incorporated into a broad range of topical dosage forms including, but not limited to, gels, oils, emulsions and the like. For instance, the suspension containing the nucleic acid-lipid particles can be formulated and administered as topical creams, pastes, ointments, gels, lotions and the like.

Once formed, the serum-stable nucleic acid-lipid particles of the present invention are useful for the introduction of nucleic acids into cells. Accordingly, the present invention also provides methods for introducing a nucleic acids (e.g., a plasmid) into a cell. The methods are carried out in vitro or in vivo by first forming the particles as described above and then contacting the particles with the cells for a period of time sufficient for transfection to occur.

The nucleic acid-lipid particles of the present invention can be adsorbed to almost any cell type with which they are mixed or contacted. Once adsorbed, the particles can either be endocytosed by a portion of the cells, exchange lipids with cell membranes, or fuse with the cells. Transfer or incorporation of the nucleic acid portion of the particle can take place via any one of these pathways. In particular, when fusion takes place, the particle membrane is integrated into the cell membrane and the contents of the particle combine with the intracellular fluid.

Using the ERP assay of the present invention, the transfection efficiency of the SPLP, SNALP or other lipid-based carrier system can be optimized. More particularly, the purpose of the ERP assay is to distinguish the effect of various cationic lipids and helper lipid components of SPLPs, SNALPs or other lipid-based carrier systems based on their relative effect on binding/uptake or fusion with/destabilization of the endosomal membrane. This assay allows one to determine quantitatively how each component of the SPLP, SNALP or other lipid-based carrier system effects transfection efficacy, thereby optimizing the SPLPs, SNALPs or other lipid-based carrier systems. As explained herein, the Endosomal Release Parameter or, alternatively, ERP is defined as:

REPORTER GENE EXPRESSION/CELL SPLP UPTAKE/CELL

It will be readily apparent to those of skill in the art that any reporter gene (e.g., luciferase, β-galactosidase, green fluorescent protein, etc.) can be used. In addition, the lipid component (or, alternatively, any component of the SPLP, SNALP or lipid-based formulation) can be labeled with any detectable label provided the does inhibit or interfere with uptake into the cell. Using the ERP assay of the present invention, one of skill in the art can assess the impact of the various lipid components (e.g., cationic lipid, noncationic lipid, PEG-lipid derivatives, PEG-DAA conjugate, ATTA-lipid derivative, calcium, CPLs, cholesterol, etc.) on cell uptake and transfection efficiencies, thereby optimizing the SPLP, SNALP or other lipid-based carrier system. By comparing the ERPs for each of the various SPLPs, SNALPs or other lipid-based formulations, one can readily determine the optimized system, e.g., the SPLP, SNALP or other lipid-based formulation that has the greatest uptake in the cell coupled with the greatest transfection efficiency.

Suitable labels for carrying out the ERP assay of the present invention include, but are not limited to, spectral labels, such as fluorescent dyes (e.g., fluorescein and derivatives, such as fluorescein isothiocyanate (FITC) and Oregon Green^{θ}; rhodamine and derivatives, such Texas red, tetrarhodimine isothiocynate (TRITC), etc., digoxigenin, biotin, phycoerythrin, AMCA, CyDyes^{θ}, and the like; radiolabels, such as ³H, 125I, ³⁵S, ¹⁴C, ³²P, ³³P, etc.; enzymes, such as horse radish peroxidase, alkaline phosphatase, etc.; spectral colorimetric labels, such as colloidal gold or colored glass or plastic beads, such as polystyrene, polypropylene, latex, etc. The label can be coupled directly or indirectly to a component of the SPLP, SNALP or other lipid-based carrier system using methods well known in the art. As indicated above, a wide variety of labels can be used, with the choice of label depending on sensitivity required, ease of conjugation with the SPLP or SNALP component, stability requirements, and available instrumentation and disposal provisions.

IV. Liposomes Containing PEG-Lipid Conjugates

In addition to the SNALP and SPLP formulations described above, the PEG-lipid conjugates of the present invention can be used in the preparation of either empty liposomes or liposomes containing one or more bioactive agents as described herein including, e.g., the therapeutic products described herein. Liposomes also typically comprise a cationic lipid and a noncationic lipid. In some embodiments, the liposomes further comprise a sterol (e.g., cholesterol).

A. Liposome Preparation

A variety of methods are available for preparing liposomes as described in, e.g., Szoka et al., Ann. Rev. Biophys. Bioeng., 9:467 (1980), U.S. Pat. Nos. 4,186,183, 4,217,344, 4,235,871, 4,261,975, 4,485,054, 4,501,728, 4,774,085, 4,837,028, 4,946,787, PCT Publication No. WO 91/17424, Deamer and Bangham, Biochim. Biophys. Acta, 443:629-634 (1976); Fraley et al., Proc. Natl. Acad. Sci. USA, 76:3348-3352 (1979); Hope et al., Biochim. Biophys. Acta, 812:55-65 (1985); Mayer et al., Biochim. Biophys. Acta, 858:161-168 (1986); Williams et al., Proc. Natl. Acad. Sci., 85:242-246 (1988), the text Liposomes, Marc J. Ostro, ed., Marcel Dekker, Inc., New York, 1983, Chapter 1, and Hope et al., Chem. Phys. Lip., 40:89 (1986). Suitable methods include, but are not limited to, sonication, extrusion, high pressure/homogenization, microfluidization, detergent dialysis, calcium-induced fusion of small liposome vesicles, and ether-infusion methods, all of which are well known in the art.

One method produces multilamellar vesicles of heterogeneous sizes. In this method, the vesicle-forming lipids are dissolved in a suitable organic solvent or solvent system and dried under vacuum or an inert gas to form a thin lipid film. If desired, the film may be redissolved in a suitable solvent, such as tertiary butanol, and then lyophilized to form a more homogeneous lipid mixture which is in a more easily hydrated powder-like form. This film is covered with an aqueous buffered solution and allowed to hydrate, typically over a 15-60 minute period with agitation. The size distribution of the resulting multilamellar vesicles can be shifted toward smaller sizes by hydrating the lipids under more vigorous agitation conditions or by adding solubilizing detergents, such as deoxycholate.

Unilamellar vesicles can be prepared by sonication or extrusion. Sonication is generally performed with a tip sonifier, such as a Branson tip sonifier, in an ice bath. Typically, the suspension is subjected to severed sonication cycles. Extrusion may be carried out by biomembrane extruders, such as the Lipex Biomembrane Extruder. Defined pore size in the extrusion filters may generate unilamellar liposomal vesicles of specific sizes. The liposomes may also be formed by extrusion through an asymmetric ceramic filter, such as a Ceraflow Microfilter, commercially available from the Norton Company, Worcester Mass. Unilamellar vesicles can also be made by dissolving phospholipids in ethanol and then injecting the lipids into a buffer, causing the lipids to spontaneously form unilamellar vesicles. Also, phospholipids can be solubilized into a detergent, e.g., cholates, Triton X, or n-alkylglucosides. Following the addition of the drug to the solubilized lipid-detergent micelles, the detergent is removed by any of a number of possible methods including dialysis, gel filtration, affinity chromatography, centrifugation, and ultrafiltration.

Following liposome preparation, the liposomes which have not been sized during formation may be sized to achieve a desired size range and relatively narrow distribution of liposome sizes. A size range of about 0.2-0.4 microns allows the liposome suspension to be sterilized by filtration through a conventional filter. The filter sterilization method can be carried out on a high through-put basis if the liposomes have been sized down to about 0.2-0.4 microns.

Several techniques are available for sizing liposomes to a desired size. One sizing method is described in U.S. Pat. No. 4,737,323. Sonicating a liposome suspension either by bath or probe sonication produces a progressive size reduction down to small unilamellar vesicles less than about 0.05 microns in size. Homogenization is another method that relies on shearing energy to fragment large liposomes into smaller ones. In a typical homogenization procedure, multilamellar vesicles are recirculated through a standard emulsion homogenizer until selected liposome sizes, typically between about 0.1 and 0.5 microns, are observed. The size of the liposomal vesicles may be determined by quasi-electric light scattering (QELS) as described in Bloomfield, Ann. Rev. Biophys. Bioeng., 10:421-450 (1981). Average liposome diameter may be reduced by sonication of formed liposomes. Intermittent sonication cycles may be alternated with QELS assessment to guide efficient liposome synthesis.

Extrusion of liposome through a small-pore polycarbonate membrane or an asymmetric ceramic membrane is also an effective method for reducing liposome sizes to a relatively well-defined size distribution. Typically, the suspension is cycled through the membrane one or more times until the desired liposome size distribution is achieved. The liposomes may be extruded through successively smaller-pore membranes, to achieve gradual reduction in liposome size. For use in the present invention, liposomes having a size ranging from about 0.05 microns to about 0.40 microns are preferred. In particularly preferred embodiments, liposomes are between about 0.05 and about 0.2 microns.

In preferred embodiments, empty liposomes are prepared using conventional methods known to those of skill in the art.

B. Use of Liposomes as Delivery Vechicles

The drug delivery compositions of the present invention (e.g., liposomes, micelles, lipid-nucleic acid particles, virosomes, etc.) are useful for the systemic or local delivery of therapeutic agents or bioactive agents and are also useful in diagnostic assays.

The following discussion refers generally to liposomes; however, it will be readily apparent to those of skill in the art that this same discussion is fully applicable to the other drug delivery systems of the present invention (e.g., micelles, virosomes, nucleic acid-lipid particles (e.g., SNALP and SPLP), etc., all of which can be advantageous formed using the PEG-lipid conjugates of the present invention).

For the delivery of therapeutic or bioactive agents, the PEG-lipid-containing liposome compositions can be loaded with a therapeutic agent and administered to the subject requiring treatment. The therapeutic agents which are administered using the compositions and methods of the present invention can be any of a variety of drugs that are selected to be an appropriate treatment for the disease to be treated. Often the drug will be an antineoplastic agent, such as vincristine (as well as the other vinca alkaloids), doxorubicin, mitoxantrone, camptothecin, cisplatin, bleomycin, cyclophosphamide, methotrexate, streptozotocin, and the like. Especially preferred antitumor agents include, for example, actinomycin D, vincristine, vinblastine, cystine arabinoside, anthracyclines, alkylative agents, platinum compounds, antimetabolites, and nucleoside analogs, such as methotrexate and purine and pyrimidine analogs. It may also be desirable to deliver anti-infective agents to specific tissues using the compounds and methods of the present inveniton. The compositions of the present invention can also be used for the selective delivery of other drugs including, but not limited to, local anesthetics, e.g., dibucaine and chlorpromazine; beta-adrenergic blockers, e.g., propranolol, timolol and labetolol; antihypertensive agents, e.g., clonidine and hydralazine; anti-depressants, e.g., imipramine, amitriptyline and doxepim; anti-conversants, e.g., phenytoin; antihistamines, e.g., diphenhydramine, chlorphenirimine and promethazine; antibiotic/antibacterial agents, e.g., gentamycin, ciprofloxacin, and cefoxitin; antifungal agents, e.g., miconazole, terconazole, econazole, isoconazole, butaconazole, clotrimazole, itraconazole, nystatin, naftifine and amphotericin B; antiparasitic agents, hormones, hormone antagonists, immunomodulators, neurotransmitter antagonists, antiglaucoma agents, vitamins, narcotics, and imaging agents.

As mentioned above, cationic lipids can be used in the delivery of therapeutic genes or oligonucleotides intended to induce or to block production of some protein within the cell. Nucleic acid is negatively charged and may be combined with a positively charged entity to form an SPLP suitable for formulation and cellular delivery of nucleic acid as described above.

Another clinical application of the PEG-lipid conjugates of this invention is as an adjuvant for immunization of both animals and humans. Protein antigens, such as diphtheria toxoid, cholera toxin, parasitic antigens, viral antigens, immunoglobulins, enzymes and histocompatibility antigens, can be incorporated into or attached onto the liposomes containing the PEG-lipid conjugates of the present invention for immunization purposes.

Liposomes containing the PEG-lipid conjugates of the present invention are also particularly useful as carriers for vaccines that will be targeted to the appropriate lymphoid organs to stimulate an immune response.

Liposomes containing the PEG-lipid conjugates of the present invention can also be used as a vector to deliver immunosuppressive or immunostimulatory agents selectively to macrophages. In particular, glucocorticoids useful to suppress macrophage activity and lymphokines that activate macrophages can be delivered using the liposomes of the present invention.

Liposomes containing the PEG-lipid conjugates of the present invention and containing targeting molecules can be used to stimulate or suppress a cell. For example, liposomes incorporating a particular antigen can be employed to stimulate the B cell population displaying surface antibody that specifically binds that antigen. Liposomes incorporating growth factors or lymphokines on the liposome surface can be directed to stimulate cells expressing the appropriate receptors for these factors. Using this approach, bone marrow cells can be stimulated to proliferate as part of the treatment of cancer patients.

Liposomes containing the PEG-lipid conjugates of the present invention can be used to deliver any product (e.g., therapeutic agents, diagnostic agents, labels or other compounds) including those currently formulated in PEG-derivatized liposomes.

In certain embodiments, it is desirable to target the liposomes of this invention using targeting moieties that are specific to a cell type or tissue. Targeting of liposomes using a variety of targeting moieties, such as ligands, cell surface receptors, glycoproteins, vitamins (e.g., riboflavin) and monoclonal antibodies, has been previously described (see, e.g., U.S. Pat. Nos. 4,957,773 and 4,603,044). The targeting moieties can comprise the entire protein or fragments thereof.

Targeting mechanisms generally require that the targeting agents be positioned on the surface of the liposome in such a manner that the target moiety is available for interaction with the target, for example, a cell surface receptor. In one embodiment, the liposome is designed to incorporate a connector portion into the membrane at the time of liposome formation. The connector portion must have a lipophilic portion that is firmly embedded and anchored into the membrane. It must also have a hydrophilic portion that is chemically available on the aqueous surface of the liposome. The hydrophilic portion is selected so as to be chemically suitable with the targeting agent, such that the portion and agent form a stable chemical bond. Therefore, the connector portion usually extends out from the liposome's surface and is configured to correctly position the targeting agent. In some cases, it is possible to attach the target agent directly to the connector portion, but in many instances, it is more suitable to use a third molecule to act as a “molecular bridge.” The bridge links the connector portion and the target agent off of the surface of the liposome, thereby making the target agent freely available for interaction with the cellular target.

Standard methods for coupling the target agents can be used. For example, phosphatidylethanolamine, which can be activated for attachment of target agents, or derivatized lipophilic compounds, such as lipid-derivatized bleomycin, can be used. Antibody-targeted liposomes can be constructed using, for instance, liposomes that incorporate protein A (see, Renneisen et al., J. Bio. Chem., 265:16337-16342 (1990) I:and Leonetti et al., Proc. Natl. Acad. Sci. (USA), 87:2448-2451 (1990)). Examples of targeting moieties can also include other proteins, specific to cellular components, including antigens associated with neoplasms or tumors. Proteins used as targeting moieties can be attached to the liposomes via covalent bonds. See, Heath, Covalent Attachment of Proteins to Liposomes, 149 Methods in Enzymology 111-119 (Academic Press, Inc. 1987). Other targeting methods include the biotin-avidin system.

In some cases, the diagnostic targeting of the liposome can subsequently be used to treat the targeted cell or tissue. For example, when a toxin is coupled to a targeted liposome, the toxin can then be effective in destroying the targeted cell, such as a neoplasmic cell.

C. Use of the Liposomes as Diagnostic Agents

The drug delivery compositions, e.g., liposomes, prepared using the PEG-DAA conjugates of the present invention can be labeled with markers that will facilitate diagnostic imaging of various disease states including tumors, inflamed joints, lesions, etc. Typically, these labels will be radioactive markers, although fluorescent labels can also be used. The use of gamma-emitting radioisotopes is particularly advantageous as they can easily be counted in a scintillation well counter, do not require tissue homogenization prior to counting and can be imaged with gamma cameras.

Gamma- or positron-emitting radioisotopes are typically used, such as .⁹⁹Tc, ²⁴Na, ⁵¹Cr, ⁵⁹Fe, ⁶⁷Ga, ⁸⁶Rb, ¹¹¹In, ¹²⁵I, and ¹⁹⁵Pt as gamma-emittiing; and such as ⁶⁸Ga, ⁸²Rb, ²²Na, ⁷⁵Br, ¹²²I and ¹⁸F as positron-emitting. The liposomes can also be labelled with a paramagnetic isotope for purposes of in vivo diagnosis, as through the use of magnetic resonance imaging (MRI) or electron spin resonance (ESR). See, for example, U.S. Pat. No. 4,728,575.

D. Loading the Liposomes

Methods of loading conventional drugs into liposomes include, for example, an encapsulation technique, loading into the bilayer and a transmembrane potential loading method.

In one encapsulation technique, the drug and liposome components are dissolved in an organic solvent in which all species are miscible and concentrated to a dry film. A buffer is then added to the dried film and liposomes are formed having the drug incorporated into the vesicle walls. Alternatively, the drug can be placed into a buffer and added to a dried film of only lipid components. In this manner, the drug will become encapsulated in the aqueous interior of the liposome. The buffer which is used in the formation of the liposomes can be any biologically compatible buffer solution of, for example, isotonic saline, phosphate buffered saline, or other low ionic strength buffers. Generally, the drug will be present in an amount of from about 0.01 ng/mL to about 50 mg/mL. The resulting liposomes with the drug incorporated in the aqueous interior or in the membrane are then optionally sized as described above.

Transmembrane potential loading has been described in detail in U.S. Pat. Nos. 4,885,172, 5,059,421, and 5,171,578. Briefly, the transmembrane potential loading method can be used with essentially any conventional drug which can exist in a charged state when dissolved in an appropriate aqueous medium. Preferably, the drug will be relatively lipophilic so that it will partition into the liposome membranes. A transmembrane potential is created across the bilayers of the liposomes or protein-liposome complexes and the drug is loaded into the liposome by means of the transmembrane potential. The transmembrane potential is generated by creating a concentration gradient for one or more charged species (e.g., Na⁺, K⁺ and/or H⁺) across the membranes. This concentration gradient is generated by producing liposomes having different internal and external media and has an associated proton gradient. Drug accumulation can than occur in a manner predicted by the Henderson-Hasselbach equation.

The liposome compositions of the present invention can by administered to a subject according to standard techniques. Preferably, pharmaceutical compositions of the liposome compositions are administered parenterally, i.e., intraperitoneally, intravenously, subcutaneously or intramuscularly. More preferably, the pharmaceutical compositions are administered intravenously by a bolus injection. Suitable formulations for use in the present invention are found in Remington's Pharmaceutical Sciences, Mack Publishing Company, Philadelphia, Pa., 17th ed. (1985). The pharmaceutical compositions can be used, for example, to diagnose a variety of conditions, or treat a variety of disease states (such as inflammation, infection (both viral and bacterial infectons), neoplasis, cancer, etc.).

Preferably, the pharmaceutical compositions are administered intravenously. Thus, this invention provides compositions for intravenous administration which comprise a solution of the liposomes suspended in an acceptable carrier, preferably an aqueous carrier. A variety of aqueous carriers can be used, e.g., water, buffered water, 0.9% isotonic saline, and the like. These compositions can be sterilized by conventional, well known sterilization techniques, or may be sterile filtered. The resulting aqueous solutions may be packaged for use as is or lyophilized, the lyophilized preparation being combined with a sterile aqueous solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

The concentration of liposome compositions in the pharmaceutical formulations can vary widely, i.e., from less than about 0.05%, usually at or at least about 2-5% to as much as 10 to 30% by weight and will be selected primarily by fluid volumes, viscosities, etc., in accordance with the particular mode of administration selected. For diagnosis, the amount of composition administered will depend upon the particular label used (i.e., radiolabel, fluorescence label, and the like), the disease state being diagnosed and the judgement of the clinician, but will generally be between about 1 and about 5 mg per kilogram of body weight.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### EXAMPLES

### Example 1

### Biodistribution, Blood Clearance and Tumor Selective Gene Expression of SPLPs Comprising PEG-lipid Conjugates

A. Materials and Methods

1. Lipids and Plasmid

The cationic lipid DODAC and the PEG-CerC₂₀ were synthesized as described previously (see, Monck et al., J. Drug Target., 7:439-452 (2000); and Hafez et al., Biophys. J., 79:1438-1446 (2000)). DOPE was obtained from Northern Lipids (Vancouver, BC, Canada). The detergent octyl glucopyranoside (OGP) was obtained from Sigma-Aldrich Co. (Oakville, ON, Canada). ³H-labelled CHE was obtained from Mandel NEN Products (Guelph, ON, Canada). The pCMVluc plasmid, encoding the luciferase reporter gene under the control of the cytomegalovirus promoter, was propagated in E. coli strain DH5α and purified by standard alkaline lysis/caesium chloride density gradient centrifugation. 2. Poly(ethylene glycol)-diacylglycerol Conjugate Synthesis

The poly(ethyleneglycol)-diacylglycerol conjugates (PEG-S-DAGs) were synthesized in-house. Briefly, succinic anhydride in 5-fold excess was stirred with 2000-weight monomethoxypolyethylene glycol in pyridine. Following purification by flash column chromatography, the free carboxylic acid was converted to the acyl chloride with a 10-fold excess of oxalyl chloride. Subsequent reaction with the relevant 1,2-diacylglycerol in the presence of triethylamine gave the PEG-S-DAGs in approximate 80% overall yield.

3. SPLP Formulation

PLP were prepared as described elsewhere (see, Wheeler et al., Gene Ther., 6:271-281 (1999)). Briefly, DOPE, DODAC and PEG-CerC₂₀ or PEG-S-DAG at a molar ratio of 82.5:7.5:10 were dissolved in aqueous solutions of OGP with or without ³H-cholesteryl hexadecyl ether (1 μCi per mg of lipid). pCMVluc plasmid solution (400 μg for 10 mg of lipid) was added to a final lipid and detergent concentration of 10 mM and 200 mM respectively. The solution was dialyzed for 48 hours and unencapsulated DNA removed by anion exchange chromatography (DEAE Sepharose CL6B). Empty vesicles were then removed by one-step sucrose density ultracentrifugation. Fractions containing SPLP were consolidated and dialyzed with HBS to remove sucrose. The final sample was then concentrated down by Amicon ultrafiltration to a final DNA concentration of 0.5 mg/ml. All samples were filter sterilized (0.22 μm) prior to injection into tumor bearing mice.

4. Exchange Assay

Five sets of liposomes were prepared for the PEG-lipid exchange assay. These liposomal systems included one for each of the three PEG-lipids, DOPE:DOPS:PEG-lipid:Rh-PE:NBD-PE (48:48:2:1:1), DOPE:DOPS (50:50) (for background count), and POPC LUVs (the PEG-lipid sink). These liposomes were prepared by freeze/thaw followed by extrusion through two 0.1 μm polycarbonate filters using an Extruder from Lipex Biomembranes (Vancouver, BC). For the assay, all formulations were pre-warmed to 37° C. Each PEG-lipid sample (performed in triplicate) would contain 25 μM of the labelled LUVs containing the PEG-lipid, 75 μM (3×) of the unlabelled DOPE:DOPS LUVs, and 250 μM (10×) of the POPC lipid sink. At t=0, the liposomal formulations for each sample were mixed and the NBD fluorescence was measured using a Cary Eclipse Fluorescence Spectrophotometer (Varian Corp, Mississauga, ON) with settings of λₑₓ=465 nm, λₑₘ=517 nm, Ex & Em slit widths=5 nm, and PMT Voltage=750V. The fluorescence at time 0 is F₀. Background blank samples were prepared in duplicate containing 100 μM (4×) of the unlabelled LUVs and 250 μM (10×) of the POPC lipid sink. The fluorescence of the background blank samples at time 0 is B₀. Following the measurements at time 0, 5 mM of CaCl₂ was added to the samples (both PEG-lipid and background blanks) and the incubation at 37° C. continued. At various time points following the addition of the CaCl₂, the fluorescence of the samples and blanks was measured (Fₜ and Bₜ, respectively). At the end of the experiment (t=23 hours), Triton X-100 was added to 0.33% causing complete lysis of the liposomes (giving the 100% fluorescence value for the labeled samples) and measurements were made on the samples and blanks (F_{T} and B_{T}, respectively). From the results, the % Fusion at each time point for the various PEG-lipid systems was determined using the following equation:
%Fusion=(Fₜ-Bₜ)−(F₀-B₀)/(F_{T}-B_{T})−(F₀-B₀)×100%

5. In Vitro Transfection Assay

Neuro-2a cells were cultured in Minimum Essential Medium (MEM; Life Technologies Inc.) supplemented with 10% fetal bovine serum (FBS; Intergen, Mass., USA) at 37° C. with 5% CO₂. Cells were dispensed into 24-well plates, with each well receiving 5×10⁴cells and 1 ml of growth medium, and incubated overnight. 500 μl of transfection media (2.5 μg/well) was added to each well and the plates incubated for the stated time-points. Media was replaced and the cells cultured for a further 24 hours. The cells were washed twice with phosphate buffered saline (PBS) and stored at −70° C. until analysis. Cells were treated with 150 μl of Cell Lysis Reagent (Promega, Wis., USA), and 20 μl of the lysate assayed for luciferase activity.

6. Biodistribution, Clearance and In Vivo Transfection Assay

10 days prior to SPLP treatment, 5-week-old male A/J mice (Harlan, Ind., USA) were inoculated subcutaneously in the hind flank with 1.5×10⁶ Neuro-2a neuroblastoma cells. Materials (SPLP-200 μl total volume containing 2 mg total lipid and 100 μg total DNA) were administered by lateral tail vein injection. At appropriate time points, mice were anaesthetized and blood collected by cardiac puncture into microtainer tubes. Plasma was separated from red blood cells via centrifugation and analyzed for ³H-CHE by liquid scintillation counting using Picofluor 20 and a Beckman LS6500 (Beckman Instruments, CA, USA). Organs were harvested at the specified times and homogenized in lysing matrix tubes containing 500 μl of distilled water. 100 μl of liver lysate and 200 μl of all other lysates were assayed for radioactivity by liquid scintillation counting with Picofluor 40. For gene expression studies 20 μl of the lysate was assayed for luciferase activity.

7. Luciferase Assay

Luciferase assays were performed using the Promega Luciferase Assay reagent kit (Promega E1501) according to the manufacturers instructions. Cell lysates were assayed for luciferase activity using a 96-well microplate luminometer. A curve obtained from firefly luciferase (Roche, PQ, Canada) standard solutions was used to calibrate luminescence readings.

B. Results

1. In Vitro Lipid Exchange Assay

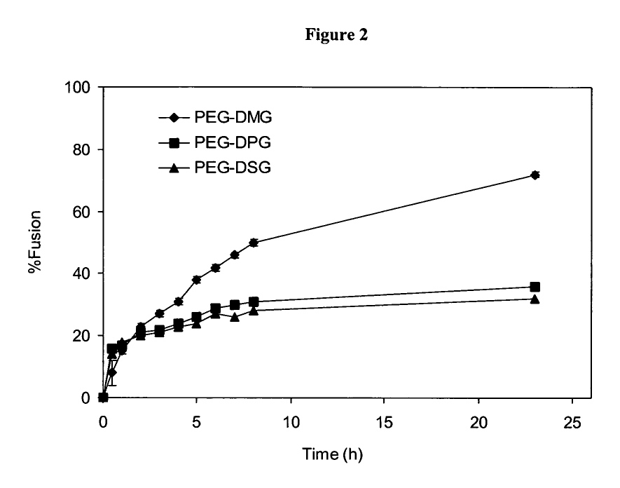

To provide additional evidence for the hypothesis that increased PEG-lipid acyl chain length correlates with increased particle stability, a lipid exchange assay was developed using fluorescent resonance energy transfer (FRET) (see, Struck et al., Biochemistry, 20:4093-4099 (1981)). PEG-lipid-containing LUVs incorporating FRET labels were mixed with unlabelled LUV in the presence of a PEG-lipid sink at 37° C. As the PEG-lipids exchange out of the LUV and are incorporated in the lipid sink the LUV are rendered increasingly fusogenic and fuse with the unlabelled LUV. A dilution of the FRET labels thus occurs and the proximity of energy donors and acceptors decreases. An increase in fluorescence is observed due to the reduced ability of the energy acceptors to quench the donors. Greater PEG-lipid diffusion results in greater fusion between LUV and an increase in fluorescence. Results are reported as a function of time over 25 hours, and entitled ‘percentage of total fusion’. The value for ‘total fusion’ is obtained at the end of the experiment by adding detergent to the sample (causing complete lysis of all vesicles) and measuring the final fluorescence signal. It can be seen from FIG. 2 that the fusion properties of SPLP containing PEG-S-DMG are vastly different from those with PEG-S-DPG and PEG-S-DSG. The final degree of fusion obtained (72%) is at least double that observed for the two longer PEG-lipids. Results were found to be significant, according to t-Test (P<0.01)

2. In Vitro Transfection Potency of SPLP Containing PEG-S-DAGs

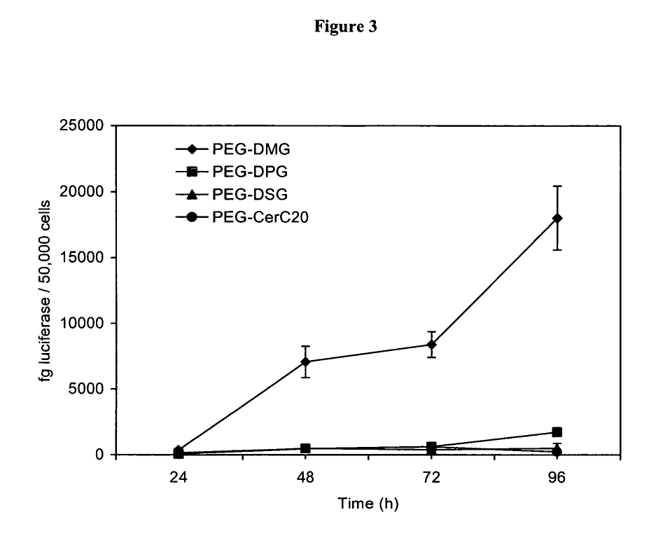

SPLP containing the short anchor PEG-S-DAGs would be expected to behave similarly to their PEG-ceramide counterparts. The PEG coating inhibits association/fusion with cell membranes (see, Harvie et al., J. Pharm. Sci., 89:652-663 (2000); and Holland et al., Biochemistry, 35:2618-2624 (1996)), therefore, transfection efficiency will be higher in systems in which it is removed more quickly/completely. To evaluate this hypothesis SPLP were prepared containing (i) DODAC, (ii) DOPE and (iii) one of the three PEG-S-DAGs or PEG-CerC₂₀ in a molar ratio of 7.5:82.5:10 and used to transfect Neuro-2a cells. Luciferase gene expression was determined over 96 hours, as shown in FIG. 3. SPLP containing the PEG-S-DAG with the shortest acyl chain, PEG-S-DMG (C₁₄), yielded the highest levels of gene expression. SPLP containing the PEG-S-DPG (C₁₆) and PEG-S-DSG (C₁₈) perform similarly to those containing the PEG-CerC₂₀. Results were found to be significant by t-Test (P<0.01)

3. Pharmacokinetics and Biodistribution of SPLP containing PEGS-DAGs

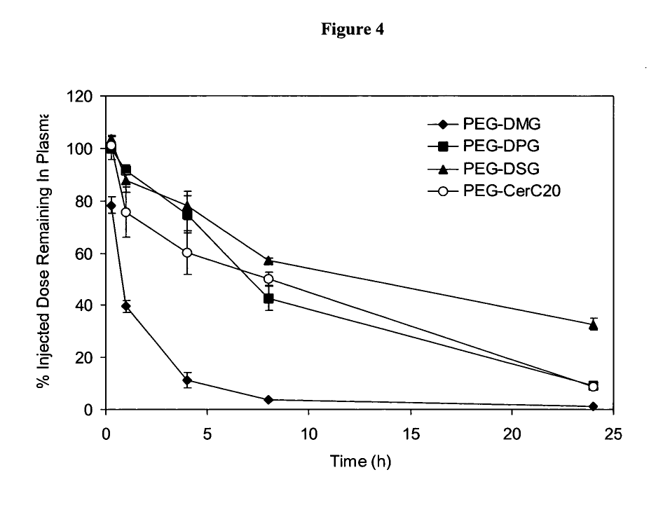

It was necessary to determine the clearance and biodistribution of SPLP containing the PEG-S-DAGs. SPLP were prepared with DODAC, DOPE, PEG-lipid (7.5:82.5:10 molar ratio),³H-CHE marker and a plasmid containing the luciferase reporter gene. SPLP were administered by a single i.v. injection in the tail vein and the percentage of injected dose remaining in the plasma determined at various timepoints. The percentage of injected dose remaining in circulation is displayed as a function of time in FIG. 4. SPLP containing the PEG-S-DMG were cleared most rapidly from the blood, with a t_{1/2} of 1 hour. Formulations containing the PEG-S-DPG and PEG-CerC₂₀ remained in the blood longer with t_{1/2} of 6 and 7 hours respectively. The PEG-S-DSG formulation exhibited the longest circulation lifetime with a t_{1/2} of 15 hours. These results are in good agreement with the results of Monck et al, who found that SPLP containing the short chain PEGCerC₁₄ had a much shorter half-life in the blood than those containing the long chain PEGCerC₂₀ (see, Monck et al., J. Drug Target., 7:439-452 (2000)).

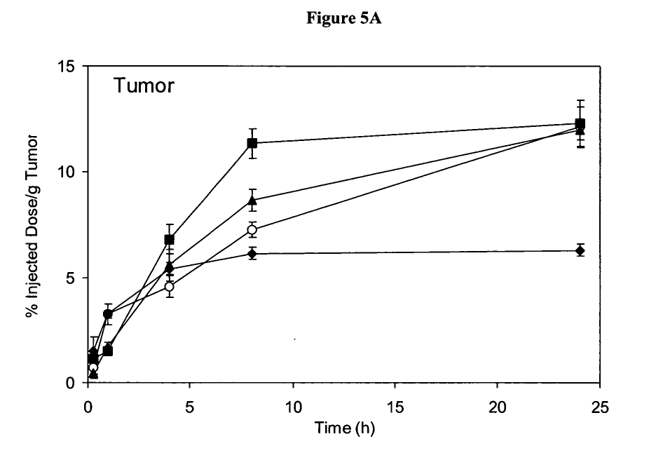

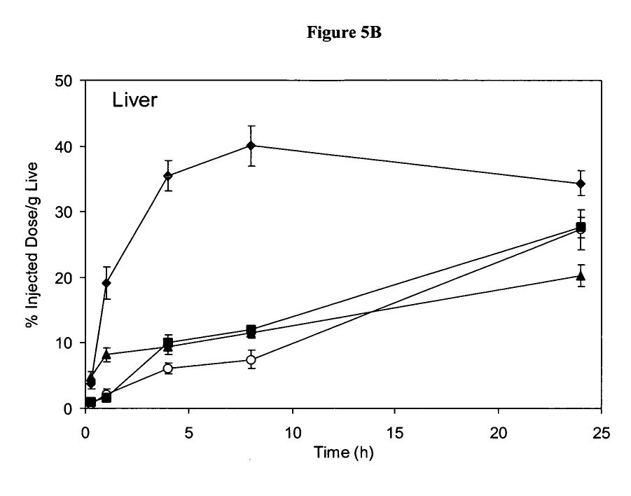

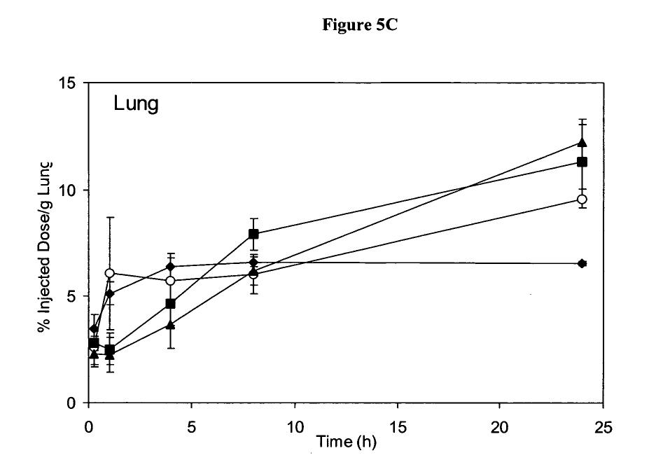

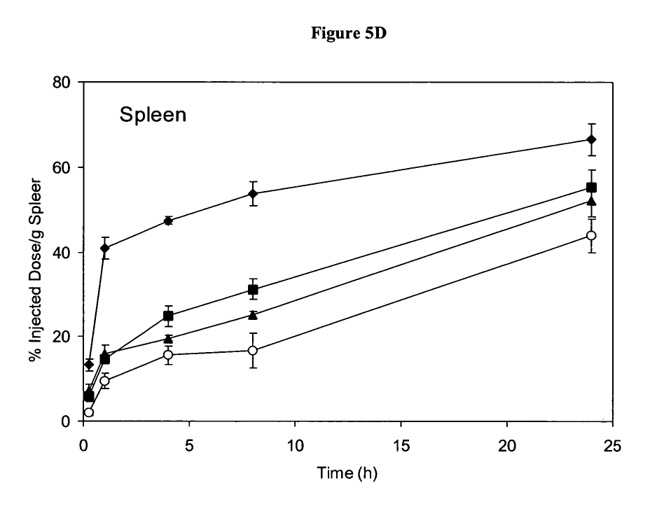

It was also of interest to confirm the ability of SPLP containing PEG-S-DAGs to bypass the first-pass organs and accumulate at the tumor site (see, Hofland et al., Pharm. Res., 14:742-749 (1997); Huang and Li, Nature Biotech., 15:620-621 (1997); Templeton et al., Nature Biotech., 15:647-652 (1997); and Thierry et al., PNAS USA, 92:9742-9746 (1995)). SPLP were administered to mice bearing subcutaneous Neuro-2A tumors. The accumulation of the SPLP in the liver, lung, spleen and tumor is shown in FIG. 5. SPLP containing the longer PEG-S-DPG and PEG-S-DSG behave similarly to those containing PEG-CerC₂₀. As expected, the PEG-S-DMG SPLP showed signs of losing their charge-shielding PEG coating more quickly. They accumulated to a greater extent in organs of the reticulo-endothelial system (RES), particularly the liver. However, all SPLP demonstrated very low levels of accumulation in the lung. SPLP with longer-chain PEG-lipids (PEG-S-DPG, PEG-S-DSG, PEG-CerC20) demonstrated increased levels of tumor accumulation when compared with PEG-S-DMG SPLP, presumably due to less accumulation in first pass organs (P<0.01). The PEG-S-DAG SPLP clearly have sufficient circulation lifetimes to facilitate passive disease site targeting.

4. Protein Expression Following Systemic Administration of SPLP Containing PEG-DAGs

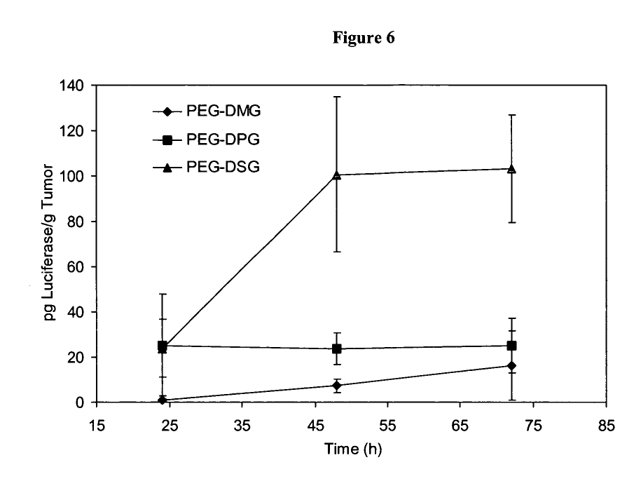

It was of obvious interest to evaluate the efficiency of protein expression both in the tumor and first pass organs. SPLP containing PEG-CerC₂₀ are known to passively target distal tumor sites and elicit transgene expression following systemic administration (see, Tam et al., Gene Ther., 7:1867-1874 (2000)). SPLP that are more rapidly cleared from the circulation have less time to accumulate at the tumor site and are expected to yield lower levels of gene expression. The time course of luciferase gene expression in the tumor resulting from administration of PEG-S-DAG SPLP is shown in FIG. 6. Gene expression would appear to increase over the 72 hour time period post-injection. Of the PEG-S-DAG SPLP formulations, those containing PEG-S-DSG yield the highest luciferase gene expression in the tumor. The amount observed is very similar to that of the PEG-CerC20 SPLP (P<0.05).

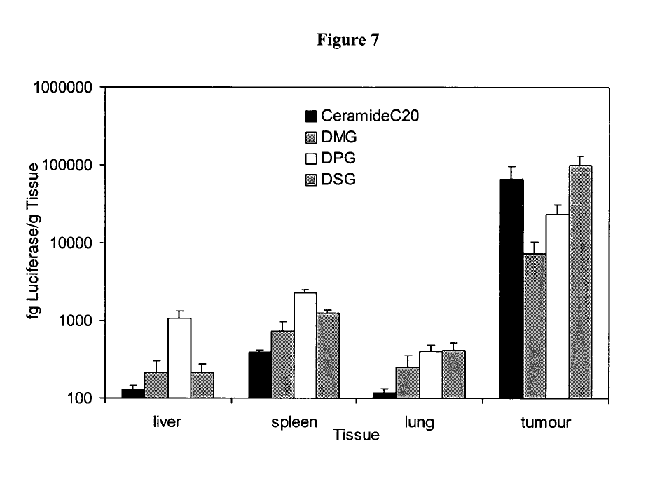

Given the pattern of SPLP biodistribution presented in FIG. 5, it was of interest to examine the resulting gene expression. Transgene expression in the tumor, lung, liver and spleen 48 hours after SPLP administration is shown in FIG. 7. Clearly, the levels of gene expression in the tumor are far greater than in first pass organs for all four types of SPLP. The PEG-S-DSG and PEG-CerC₂₀ SPLP in particular exhibit large differentials that represent from 100- to almost 1000-fold increases over the other tissues. It would be expected that for each tissue type, biodistribution would be reflected in gene expression. For example, SPLP containing PEG-S-DSG and PEG-CerC₂₀ exhibit similar biodistribution profiles and accumulate at the tumor site in similar amounts. Thus, resulting tumor transfection may be expected to be comparable. This is indeed the case.

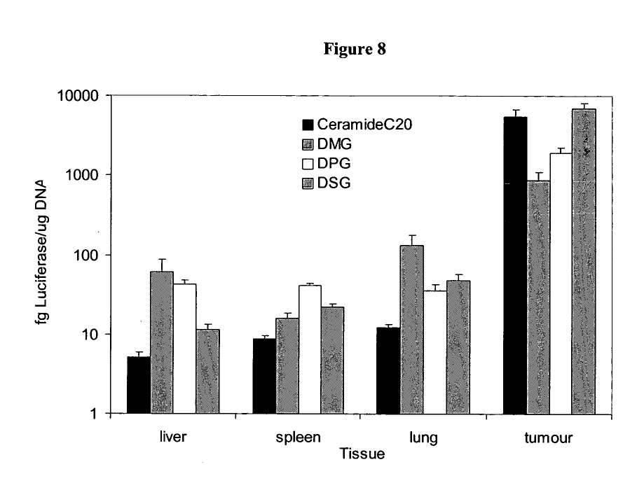

A measure of the relative potency of SPLP in the different tissue types can be obtained by evaluating gene expression as a function of the amount of SPLP accumulation. FIG. 8 illustrates this relationship. The liver and spleen, despite accumulating large concentrations of SPLP, demonstrate very modest transgene expression. Intriguingly, this analysis shows that SPLP are up to 1000-fold more potent when transfecting tumor tissue than when transfecting cells of the lung, liver and spleen.

C. Discussion

This study demonstrates that PEG-S-DAGs can be successfully incorporated in SPLP and the resulting particles behave in a manner similar to those containing PEG-ceramides. PEG-S-DAGs are more easily and less expensively synthesized than their ceramide counterparts and are easier to purify. PEG-S-DAGs with three different lipid domains of varying lengths were synthesized and incorporated into SPLP. In this study, the rate and extent to which the different PEG molecules dissociate from the bilayer was modeled using an in vitro exchange assay, providing evidence for the proposed mode of action of these compounds. The in vitro and in vivo transfection efficiency, biodistribution and serum clearance of SPLP containing PEG-lipid conjugates of different lengths were evaluated.

In the exchange assay, vesicles containing PEG-S-DMG were clearly shown to become more fusogenic over time as the PEG-lipid dissociated from the particle. This is due to the uncovering of the fusogenic lipid bilayer as the outer PEG coating is removed. After 23 hours at 37° C., the degree of fusion in the sample had reached 72%. The PEG-S-DPG and PEG-S-DSG exhibited much reduced fusion profiles, with these samples achieving 36% and 32% fusion by the end of the experiment. This is good evidence of the speed and extent to which the different PEG-lipids are exchanging from the bilayer. It is thought that lipidic compounds are held in the bilayer of lipid vesicles (such as SPLP) predominately by hydrophobic interactions between their hydrophobic domains (see, Massey et al., Biochimica Et Biophysica Acta, 794:274-280 (1984)). Thus, a longer PEG-S-DAG acyl chain will have stronger forces securing it to the bilayer, and the molecule will remain bound for a greater period of time. It is true that other factors may influence rate of diffusion, such as lipid head group chemistry (see, Homan and Pownall, Biochimica Et Biophysica Acta, 938:155-166 (1988)), or the presence (or absence) of specific proteins (see, Pownall and Hamilton, Acta Physiol. Scand., 178:357-365 (2003)). However the exchange assay result correlates well with investigations into criteria that are most likely to be controlled by PEG dissociation—in vitro gene expression and in vivo circulation lifetime. In these experiments too, the PEG-S-DMG particles behave in a markedly different fashion to the PEG-S-DPG and PEG-S-DSG, as described below.

When examining gene expression in vitro, factors such as blood components and clearance by the liver and lung need not be considered. Since the dissociation of the PEG is required for the particle to become transfection competent, gene expression is related entirely to the rate at which the PEG-molecule is removed from the SPLP bilayer. As predicted, when examining the in vitro transfection efficiency of the different PEG-S-DAG containing SPLP, the potency of the short chain PEG-S-DMG formulation was found to be greatest. This result correlates with the present exchange assay, as well as the findings of other authors (see, Mok et al., Biochim. Biophys. Acta-Biomembr., 1419:137-150 (1999); and Wheeler et al., Gene Ther., 6:271-281 (1999)), who have found SPLP containing shorter chain PEG-ceramides to be more transfection competent in vitro. SPLP containing the longer PEG-S-DPG and PEG-S-DSG perform similarly to those containing the PEG-CerC₂₀.

The behavior of SPLP in vivo is considerably more complex. PEG coatings conceal the positive charge of SPLP and prevent interaction of the fusogenic DOPE with other lipid membranes (e.g., cellular membranes, other SPLP etc.) (see, Harvie et al., J. Pharm. Sci., 89:652-663 (2000); and Holland et al., Biochemistry, 35:2618-2624 (1996)). This lends the particle characteristics that allow for an extended circulation lifetime in the blood. The longer the PEG coating remains intact on the surface of the SPLP, the longer the particle's systemic half-life. This observation has been verified by other researchers, using PEG-ceramides (see, Monck et al., J. Drug Target., 7:439-452 (2000); and Webb et al., Biochim. Biophys. Acta-Biomembr., 1372:272-282 (1998)). Even very short PEG ceramides, such as the C₈, substantially increase the circulation time of SPLP when compared to DNA:lipid complexes (see, Tam et al., Gene Ther., 7:1867-1874 (2000)). PEG-S-DAGs impart similar characteristics. PEG-S-DMG formulations are cleared with a half-life of less than an hour. Increasing the length of the acyl chains of the PEG-S-DAGs increases the length of time that the SPLP will remain in the blood compartment. As with the in vitro transfection experiment, it is the PEG-S-DSG that most closely resembles the PEG-CerC₂₀ control.

PEG association and circulation lifetime have a direct effect on the biodistribution of SPLP. Unlike lipoplex systems, the amount of PEG-S-DAG SPLP accumulation in the lung is extremely low, corresponding to approximately 1% of the overall injected dose. Accumulation in the liver and spleen is somewhat higher. Extrapolating the results of the exchange assay, one would expect PEG-S-DMG SPLP to lose the stabilizing PEG coating more quickly, resulting in greater accumulation in organs of the RES, as is the case (FIG. 5). To take advantage of passive targeting, particles must bypass these organs and remain in the circulation long enough to encounter the fenestrations in the tumor vasculature. SPLP containing the longer chain PEG compounds are therefore most successful. In this regard, PEG-S-DSG SPLP once more give the most similar results to the PEG-CerC₂₀ control.

The gene expression profiles of the SPLP are significant. Systemic delivery and subsequent gene expression in distal tumors has already been reported (see, Tam et al. and Monck et al., supra). MacLachlan et al first demonstrated how the expression obtained with PEG-CerC₂₀ SPLP was highly selective for the tumor by comparison with the other organs (see, Fenske et al., Methods Enzymol., 346:36-71 (2002)). These results with the PEG-S-DAGs are similar to those obtained with the PEG-CerC₂₀. Large differentials between transfection of the tumor and other organs are seen, in some cases up to 1000-fold. Due to slightly reduced accumulation of PEG-S-DMG SPLP in the tumor, transfection would be expected to be somewhat lower, as is the case. Of greatest interest was the analysis of relative SPLP potency at each organ site. It can be seen that the high transfection efficiency at the tumor is not simply a result of greater accumulation at this site; in fact, SPLP accumulation at the tumor is only marginally greater than at the lung, and far less than the liver or spleen. Rather, it appears the SPLP collecting at the tumor site are more efficient at transfecting this tissue. The explanation may incorporate several factors.

The mitotic index (i.e., the speed at which the cells proliferate) may play a role in the preferential transfection of tumor tissue. It is known that nuclear delivery of non-viral vectors is facilitated by the breakdown of the nuclear envelope, and occurs during the prometaphase at the beginning of the cell's M phase (see, Mortimer et al., Gene Ther., 6:403-411 (1999)). In vitro experiments using lipoplexes and SPLP showed that transfection efficiency was reduced by a factor of 20 in a cell population arrested in the GI phase. This has implications for the SPLP mediated transfection of quiescent tissue. Until the nuclear membrane is disrupted, the DNA, now stripped of the protective lipid shell, will remain in the cytoplasm where it is subject to degradation by cytoplasmic nucleases. Lechardeur et al reported that the half-life of plasmid DNA in the cytoplasm of HeLa and Cos cells is 50-90 minutes (see, Lechardeur et al., Gene Ther., 6:482-497 (1999)). Hence, in more quickly dividing cells, such as tumor cells, there is less chance of the plasmid being the subjected of enzymatic breakdown. With respect to gene expression, this would manifest itself as tumor cells being transfected far more efficiently than other, more quiescent tissue.

Tam et al also reported that accumulation of SPLP in the liver does not yield significant gene expression (see, Tam et al., supra). Analysis indicated that, despite high concentrations of the ³H-CHE marker, there was actually very little intact plasmid in the liver. They postulated that this might reflect relatively rapid breakdown of the SPLP and its' associated plasmid following uptake into liver phagocytes (Kuppfer cells). These cells are known to play a leading role in the clearance of liposomal systems from the circulation (see, Roerdink et al., Biochimica Et Biophysica Acta, 677:79-89 (1981)).

It is also true that perturbations in transcriptional efficiency may have an effect on gene expression, both directly and indirectly (see, Cox and Goding, Br. J. Cancer, 63:651-662 (1991)). The altered phenotype of the tumor cell could simply result in more efficient transcription of the transgene. Alternatively, disregulated expression of certain genes common in transformed tissue may lead to biophysical/biochemical changes in the cell, such as increased rates of endocytosis or over-expression of cell surface receptors. Both of these could lead to more of the SPLP actually being internalized and increase the chance of transgene expression.

In summary, the results presented here show that PEG-S-DAGs can be substituted for PEG-ceramides to produce SPLP using the detergent dialysis procedure. The resulting SPLP show the same relationship between the PEG anchor chain length and transfection/pharmacokinetic behaviour. These results support the theory of diffusible PEG-lipids that provide serum stability and long circulation lifetimes. Further evidence for this argument is provided by showing that shorter chain PEG-lipids exchange more completely from the bilayer in an in vitro exchange assay. PEG-S-DAGs are easier to synthesize and purify than PEG-ceramides and present an attractive alternative for the production of SPLP. It is concluded that PEG-S-DSG is a functionally effective replacement for PEG-CerC₂₀ in SPLP formulations for systemic tumor delivery and gene expression.

### Example 2

### Expression of Nucleic Acids Encapsulated in SPLP Comprising PEG-dialkyloxypropyl Conjugates

This example describes experiments comparing expression of nucleic acids encapsulated in SPLP comprising PEG-diacylglycerol conjugates versus SPLP comprising PEG-dialkyloxypropyl conjugates. All SPLP formulations comprise a plasmid encoding luiferase under the control of the CMV promoter (pLO55).

| | | | | | | | Time | |
|---|---|---|---|---|---|---|---|---|
| | # | | | | | # | after final | |
| Group | Mice | Cell | Route | Treatment | Route | Doses | injection | Assay* |
| A | 6 | Neuro-2a | SC | PBS | IV | 1 | 48 hrs | Body weights, |
| B | 6 | Neuro-2a | SC | SPLP PEG- | IV | 1 | 48 hrs | Blood analyses, |
| | | | | DSG | | | | Luciferase |
| C | 6 | Neuro-2a | SC | SPLP PEG- | IV | 1 | 48 hrs | activity |
| | | | | DSPE | | | | |
| D | 6 | Neuro-2a | SC | SPLP PEG- | IV | 1 | 48 hrs | |
| | | | | CeramideC20 | | | | |
| E | 6 | Neuro-2a | SC | SPLP PEG-A- | IV | 1 | 48 hrs | |
| | | | | DSA | | | | |
| F | 6 | Neuro-2a | SC | SPLP PEG-C- | IV | 1 | 48 hrs | |
| | | | | DSA | | | | |
| G | 6 | Neuro-2a | SC | SPLP PEG-S- | IV | 1 | 48 hrs | |
| | | | | DSA | | | | |

All SPLP formulations contained pLO55 and DSPC:Chol:DODMA:PEG-Lipid (20:55:15:10). The following formulations were made:

A: PBS (pH 7.4).

B: L055 PEG-DSG SPLP, 0.50 mg/ml.

C: L055 PEG-DSPE SPLP, 0.50 mg/ml.

D: L055 PEG-CeramideC20 SPLP, 0.50 mg/ml.

E: L055 PEG-A-DSA SPLP, 0.50 mg/ml.

F: L055 PEG-C-DSA SPLP, 0.50 mg/ml.

G: L055 PEG-S-DSA SPLP, 0.50 mg/ml.

| | No. | Seedling | | Injection | Collection |
|---|---|---|---|---|---|
| Group | Mice | date | Treatment | date | date |
| A | 6 | Day 0 | PBS | Day 13 | Day 15 |
| B | 6 | Day 0 | SPLP PEG-DSG | Day 13 | Day 15 |
| C | 6 | Day 0 | SPLP PEG-DSPE | Day 13 | Day 15 |
| D | 6 | Day 0 | SPLP PEG- | Day 13 | Day 15 |
| | | | CeramideC20 | | |
| E | 6 | Day 0 | SPLP PEG-A-DSA | Day 13 | Day 15 |
| F | 6 | Day 0 | SPLP PEG-C-DSA | Day 13 | Day 15 |
| G | 6 | Day 0 | SPLP PEG-S-DSA | Day 13 | Day 15 |

1.5×10⁶ Neuro2A cells in 50 μl PBS were subcutaneously administered to each mouse on day 0. On day 13, mice were randomized and treated with one dose of an SPLP formulation or PBS by intravenous (IV) injection. Dose amounts are based on body weight measurements taken on the day of dosing. 48 hours after SPLP administration, the mice were weighed and sacrificed, their blood was collected, and the following tissues were collected, weighed, immediately frozen and stored at −80° C. until further analysis: tumor, liver (cut in 2 halves), lungs, spleen and heart.

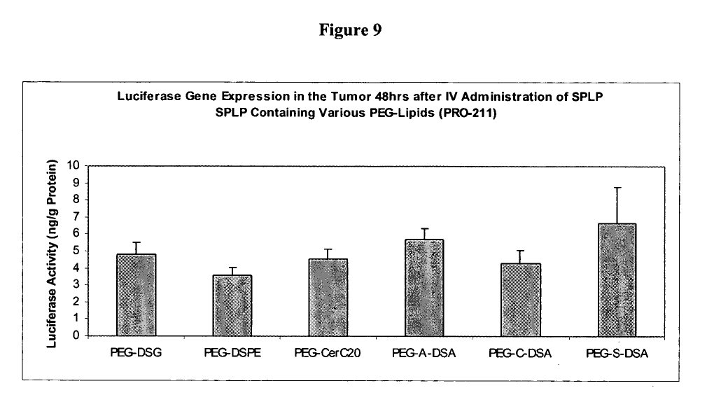

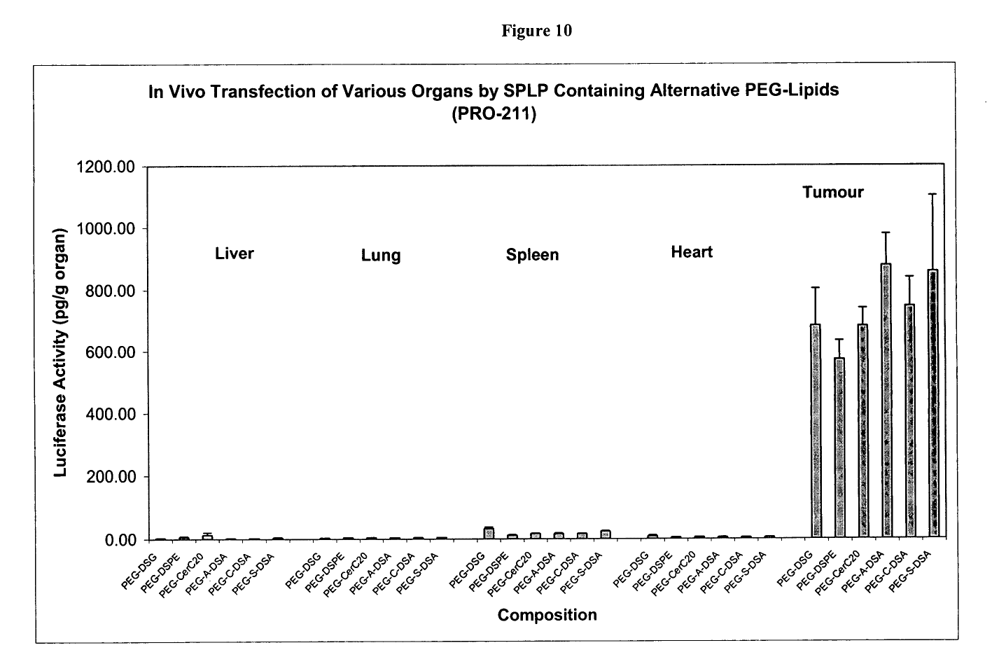

Gene expression in collected tissues was determined by assaying for enzymatic activity of expressed luciferase reporter protein. The results are shown in FIGS. 9 and 10.

The results demonstrate that 48 hours following i.v. injection of the SPLP, the transfection levels in the tumor by the SPLP comprising PEG-dialkyloxypropyl conjugates are substantially similar to those by SPLP comprising PEG-diacylglycerol conjugates. The amount of gene expression in the organs (liver, lung, spleen, and heart) of the mice injected with the SPLP comprising PEG-dialkyloxypropyl conjugates is also substantially similar to that of SPLP comprising PEG-diacylglycerol conjugates.

### Example 3

### Expression of Nucleic Acids Encapsulated in SPLP Comprising PEG-dialkyloxypropyl Conjugates

This examples describes experiments comparing expression of nucleic acids encapsulated in SPLP comprising PEG-dialkyloxypropyl conjugates. All SPLP formulations comprise a plasmid encoding luiferase under the control of the CMV promoter (pLO55)

| | # | | | | | # | | |
|---|---|---|---|---|---|---|---|---|
| Group | Mice | Tumor | Route | Treatment | Route | Doses | Timepoint | ASSAY*** |
| A | 4 | Neuro- | SC | PBS | IV | 1 | 48 hrs | Body weights, |
| | | 2a | | | | | | Blood analyses, |
| B | 5 | Neuro- | SC | SPLP PEG-DSG | IV | 1 | 48 hrs | Luciferase |
| | | 2a | | | | | | activity |
| C | 5 | Neuro- | SC | SPLP PEG-A-DSA | IV | 1 | 48 hrs | |
| | | 2a | | | | | | |
| D | 5 | Neuro- | SC | SPLP PEG-A-DPA | IV | 1 | 48 hrs | |
| | | 2a | | | | | | |
| E | 5 | Neuro- | SC | SPLP PEG-A-DMA | IV | 1 | 48 hrs | |
| | | 2a | | | | | | |

The lipids (DSPC:CHOL:DODMA:PEG-Lipid ) were present in the SPLP in the following molar ratios (20:55:15:10). The following formulations were made:

A: PBS sterile filtered, 5 mL.

B: pL055-SPLP with PEG-DSG, 2 mL at 0.50 mg/mL.

C: pL055-SPLP with PEG-A-DSA, 2 mL at 0.50 mg/mL.

D: pL055-SPLP with PEG-A-DPA, 2 mL at 0.50 mg/mL.

E: pL055-SPLP with PEG-A-DMA, 2 mL at 0.50 mg/mL.

| | # | Seeding | | Injection | Collection |
|---|---|---|---|---|---|
| Group | Mice | date | Treatment | date | date |
| A | 4 | Day 0 | PBS | Day 12 | Day 14 |
| B | 5 | Day 0 | SPLP PEG-DSG | Day 12 | Day 14 |
| C | 5 | Day 0 | SPLP PEG-A-DSA | Day 12 | Day 14 |
| D | 5 | Day 0 | SPLP PEG-A-DPA | Day 12 | Day 14 |
| E | 5 | Day 0 | SPLP PEG-A-DMA | Day 12 | Day 14 |

1.5×10⁶ Neuro2A cells were administered to each mouse on day 0. When the tumors were of a suitable size (200-400 mm3), mice were randomized and treated with one dose of an SPLP formulation or PBS by intravenous (IV) injection. Dose amounts are based on body weight measurements taken on the day of dosing. 48 hours after SPLP administration, the mice were sacrificed, their blood was collected, and the following tissues will be collected weighed, immediately frozen and stored at −80° C. until further analysis: tumor, liver (cut in 2 halves), lungs, spleen and heart.

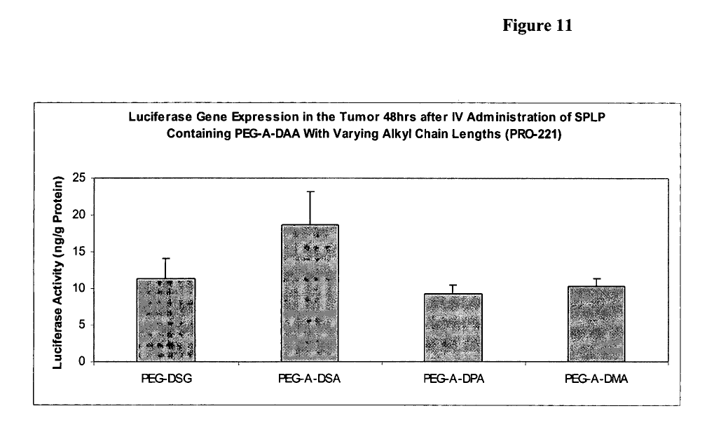

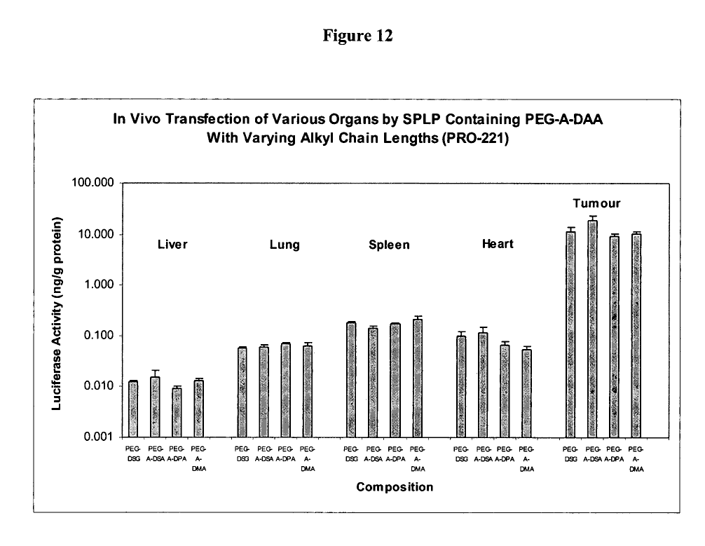

Gene expression in collected tissues was determined by assaying for enzymatic activity of expressed luciferase reporter protein. The results are shown in FIGS. 11 and 12.

The results indicate that SPLP comprising PEG-dialkyloxypropyls (i.e., PEG-DAA) can conveniently be used to transfect distal tumor to substantially the same extent as SPLP comprising PEG-diacylglycerols. Moreover, the transfection levels seen with SPLP containing PEG-dialkyglycerols are similar to those seen with SPLP containing PEG-diacylglycerols (e.g., PEG-DSG). The results also demonstrate that very little transfection occurred in non-tumor tissues. Moreover, the SPLP comprising PEG-dialkyloxypropyls exhibit reduced toxicity compared to other SPLP formulations.

### Example 4

### Expression of Nucleic Acids Encapsulated in SPLP and pSPLP Comprising PEG-dialkyloxypropyl Conjugates

This example describes experiments comparing expression of nucleic acids encapsulated in SPLP comprising PEG-dialkyloxypropyls versus PEI condensed DNA (pSPLP) in comparison to the SPLP.

| | | | | | Timepoint | |
|---|---|---|---|---|---|---|
| | # | | | | after final | |
| Group | Mice | Cell | Treatment | Route | inection | Assay* |
| A | 4 | SC Neuro- | 1 dose PBS | IV | 48 hrs | Luciferase |
| | | 2a | | | | activity |
| B | 4 | SC Neuro- | 1 dose L055-pSPLP PEG- | IV | 48 hrs | |
| | | 2a | DSG | | | |
| C | 4 | SC Neuro- | 1 dose L055-pSPLP PEG- | IV | 48 hrs | |
| | | 2a | DPG | | | |
| D | 4 | SC Neuro- | 1 dose L055-pSPLP PEG- | IV | 48 hrs | |
| | | 2a | DMG | | | |
| E | 4 | SC Neuro- | 1 dose L055-pSPLP PEG- | IV | 48 hrs | |
| | | 2a | A-DSA | | | |
| F | 4 | SC Neuro- | 1 dose L055-pSPLP PEG- | IV | 48 hrs | |
| | | 2a | A-DPA | | | |
| G | 4 | SC Neuro- | 1 dose L055-pSPLP PEG- | IV | 48 hrs | |
| | | 2a | A-DMA | | | |
| H | 4 | SC Neuro- | 1 dose L055-SPLP PEG- | IV | 48 hrs | |
| | | 2a | A-DSA | | | |
| I | 4 | SC Neuro- | 1 dose L055-SPLP PEG- | IV | 48 hrs | |
| | | 2a | A-DPA | | | |
| J | | SC Neuro- | 1 dose L055-SPLP PEG- | IV | 48 hrs | |
| | | 2a | A-DMA | | | |
| K | 4 | SC Neuro- | 1 dose L055-SPLP PEG- | IV | 48 hrs | |
| | | 2a | A-DMA | | | |
| | | | at 20 mg pDNA/Kg | | | |

All formulations contained DSPC:Chol:DODMA:PEG-DAG (20:55:15:10). The following formulations were made:

A: PBS (pH 7.4).

B: L055 PEG-DSG pSPLP, 0. 5 mg/ml.

C: L055 PEG-DPG pSPLP, 0.43 mg/ml.

D: L055 PEG-DMG pSPLP, 0.5 mg/ml.

E: L055 PEG-A-DSA pSPLP, 0.5 mg/ml.

F: L055 PEG-A-DPA pSPLP, 0.5 mg/ml.

G: L055 PEG-A-DMA pSPLP, 0.5 mg/ml.

H: L055 PEG-A-DSA SPLP, 0.5 mg/ml.

I: L055 PEG-A-DPA SPLP, 0.5 mg/ml.

J: L055 PEG-A-DMA SPLP, 0.5 mg/ml.

K. L055 PEG-A-DMA SPLP, 2.1 mg/ml.

1.5×10⁶ Neuro2A cells in 50 μl PBS were subcutaneously administered to each mouse on day 0. On day 13, mice were randomized and treated with one dose of an SPLP formulation or PBS by intravenous (IV) injection. Dose amounts are based on body weight measurements taken on the day of dosing. 48 hours after SPLP administration, the mice were weighed and sacrificed, their blood was collected, and the following tissues were collected, weighed, immediately frozen and stored at −80° C. until further analysis: tumor, liver (cut in 2 halves), lungs, spleen and heart.

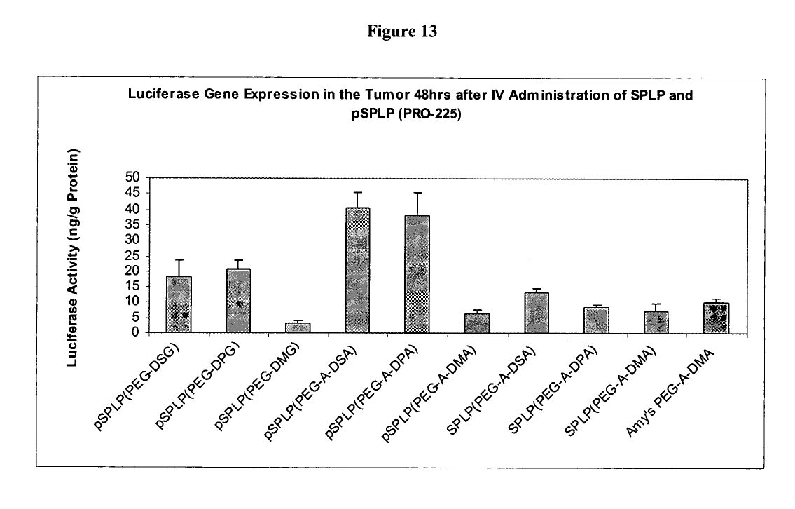

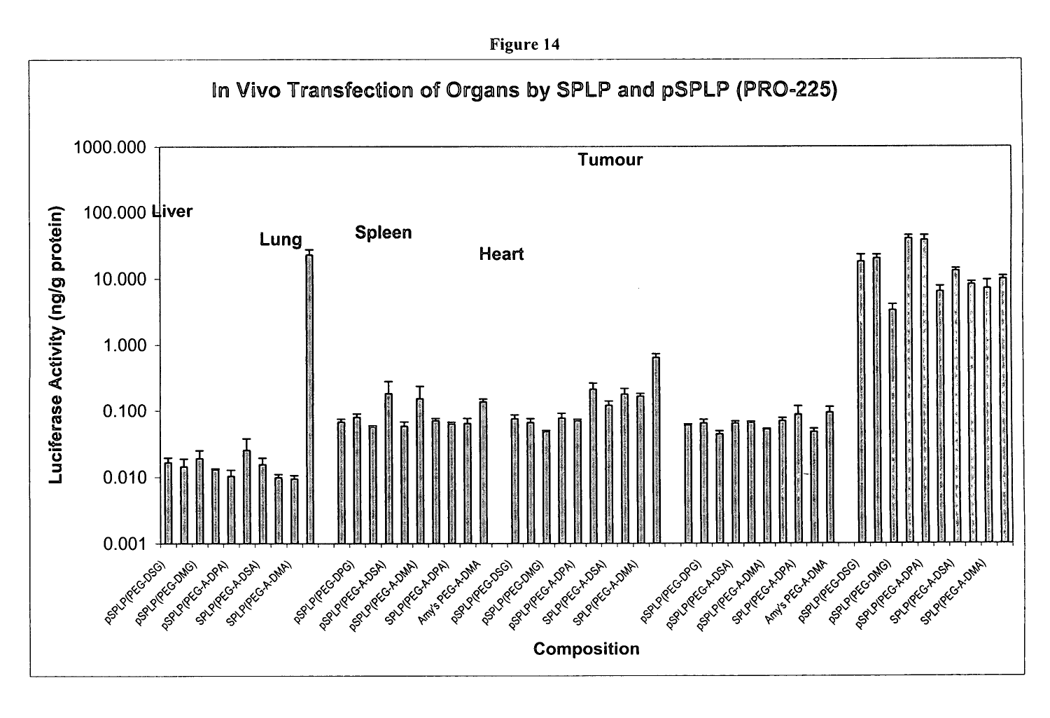

Gene expression in collected tissues was determined by assaying for enzymatic activity of expressed luciferase reporter protein. The results are shown in FIGS. 13 and 14.

| | No. | Tumor | SPLP | |
|---|---|---|---|---|
| Group | Mice | SC | Treatments | Termination |
| A | 4 | Day 0 | Day 12 | Day 14 |
| B | 4 | Day 0 | Day 12 | Day 14 |
| C | 4 | Day 0 | Day 12 | Day 14 |
| D | 4 | Day 0 | Day 12 | Day 14 |
| F | 4 | Day 0 | Day 12 | Day 14 |
| F | 4 | Day 0 | Day 12 | Day 14 |
| G | 4 | Day 0 | Day 12 | Day 14 |
| H | 4 | Day 0 | Day 12 | Day 14 |
| I | 4 | Day 0 | Day 12 | Day 14 |
| J | 4 | Day 0 | Day 12 | Day 14 |

The results indicate that the presence of the short chain PEG-lipids (i.e., PEG-DMG and PEG-A-DMA) in pSPLP results in an approximate 5-10 fold decrease in tumor transfection compared to the long chained versions (i.e., PEG-DSG and PEG-A-DSA).

Taken together these results indicate that the enhancement in tumor transfection seen with the pSPLP (C₁₈ PEG-lipids) over the SPLP is completely abolished when the pSPLP contains the C₁₄ PEG-lipids. This could be due to a number of factors: (1) a decrease in stability of the pSPLP when the PEG-lipid leaves the bilayer of the pSPLP, (2) an increase in charge upon PEG-lipid removal, or (3) the conditions for the C14 PEG-lipids have not been optimized (e.g,amount of anionic lipid in the bilayer). Further experiments will need to be performed to determine which of these if any is the issue. Also, the activities in the other organs tested were quite low for all the systems. Interestingly, a 20 mg/kg dose of PEG-A-DMA SPLP gave comparable levels of luciferase gene expression in the tumor as the 5 mg/kg dose, but much higher gene expression in the liver compared to the same 5 mg/kg dose.

### Example 5

### Silencing of Gene expression with SNALPS

This example illustrates silencing of gene expression in Neuro 2A tumor bearing mice after co-administration of SPLPs containing a plasmid encoding luciferase under the control of the CMV promoter and SNALPs containing anti-luciferase siRNA.

| | # | | | | | | # |
|---|---|---|---|---|---|---|---|
| Group | Mice | Tumor | Route | Treatment | Timepoint | Route | Doses |
| 1 | 3 | Neuro- | SQ | PBS/PBS | 48 h | IV | 1 |
| 24A | 4 | 2a | | L055-SPLP/PBS mix | | | |
| 24B | 4 | | | L055-SPLP/anti-luc siRNA | 24 h | | |
| | | | | liposomes mix | | | |
| 48A | 4 | | | L055-SPLP/PBS mix | | | |
| 48B | 4 | | | L055-SPLP/anti-luc siRNA | 48 h | | |
| | | | | liposomes mix | | | |
| 72A | 4 | | | L055-SPLP/PBS mix | | | |
| 72B | 4 | | | L055-SPLP/anti-luc siRNA | 72 h | | |
| | | | | liposomes mix | | | |

| | # | Seeding | | | | Injection | Collection |
|---|---|---|---|---|---|---|---|
| Group | Mice | Date | Route | IV Treatment | Timepoint | Route | Date |
| 1 | 3 | Day 0 | SQ | PBS/PBS | 48 h | Day 13 | Day 15 |
| 24A | 4 | | | L055-SPLP/PBS mix | | Day 14 | |
| 24B | 4 | | | L055-SPLP/anti-luc | 24 h | Day 14 | |
| | | | | siRNA liposomes mix | | | |
| 48A | 4 | | | SQL055-SPLP/PBS mix | | Day 13 | |
| 48B | 4 | | | L055-SPLP/anti-luc | 48 h | Day 13 | |
| | | | | siRNA liposomes mix | | | |
| 72A | 4 | | | L055-SPLP/PBS mix | | Day 12 | |
| 72B | 4 | | | L055-SPLP/anti-luc | 72 h | Day 12 | |
| | | | | siRNA liposomes mix | | | |

36 male A/J mice (Jackson Laboratories) were seeded subcutaneously with Neuro 2A cells at a dose of 1.5×10⁶ cells in a total volume of 50 μL phosphate buffered saline on day zero. Once tumors reached appropriate size (typically on day 9 or later), 200-240 μl PBS, SPLP, or SNALP formulations (100 μg nucleic acid total) prepared as described in Example 6 above, were administered intravenously. 24, 48, or 72 after administration of PBS, SPLP or a mixture of SPLP and SNALP, mice were sacrificed and organs (e.g., liver, lung, spleen, kidney, heart) and tumors were collected and evaluated for luciferase activity.

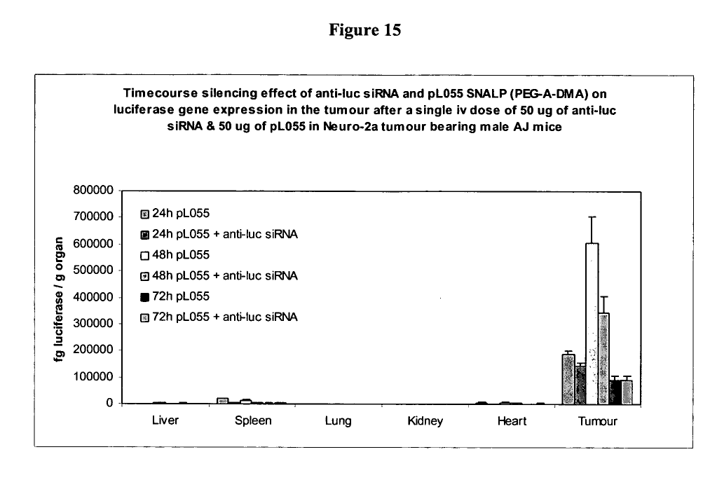

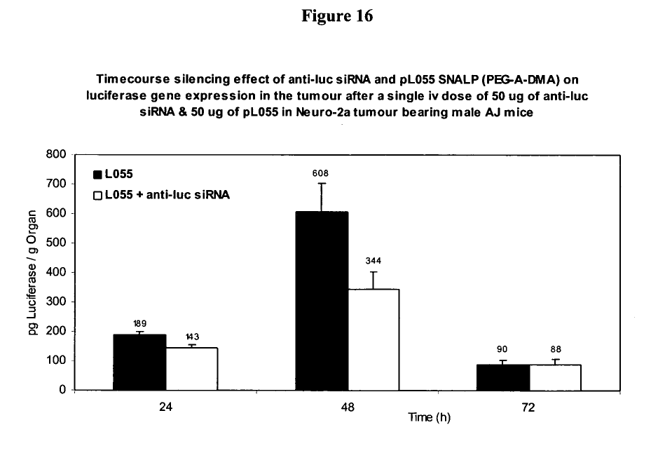

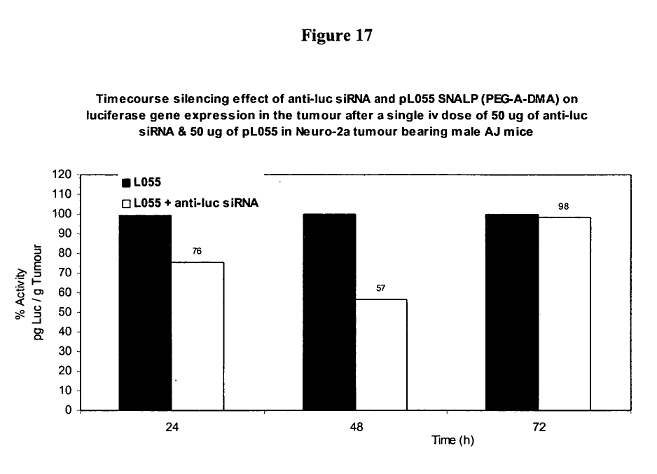

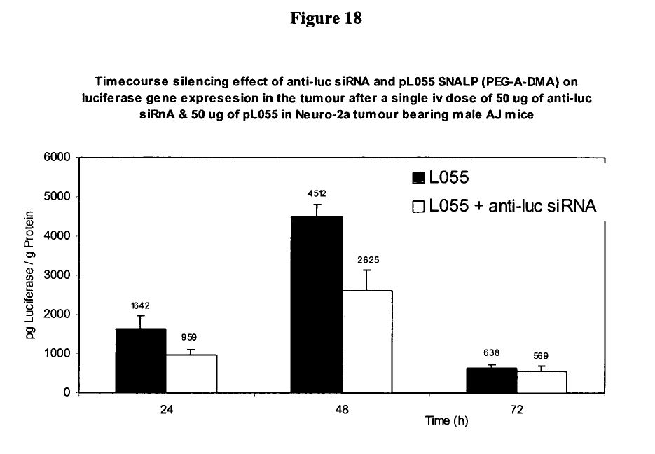

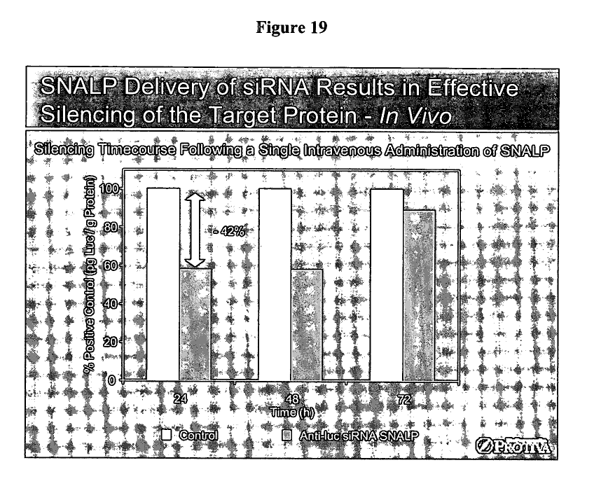

Co-administration of pL055 SPLP and anti-luc siRNA SNALP (both containing PEG-A-DMA) maximally decreases luciferase gene expression by 40% forty-eight hours after a single iv dose. The results are shown in FIGS. 15-19.

### Example 6

### Uptake of SPLP Comprising PEG-DAA Conjugates

This example illustrates the uptake of SPLP comprising PEG-DAA conjugates by mammalian cells in vitro. The SPLP formulations set forth in the table below were labeled with ³H-CHE and incubated on the cells at either 4° C. or 37° C. for 24 hours. The SPLP comprised either 2, 4, or 10 mol % PEG-C-DMA.

| | Mol % |
|---|---|
| | (DSPC:Chol:PEG-C-DMA:DODMA) |
| A | 20:50:10:15 |
| B | 20:61:4:15 |
| C | 20:63:2:15 |

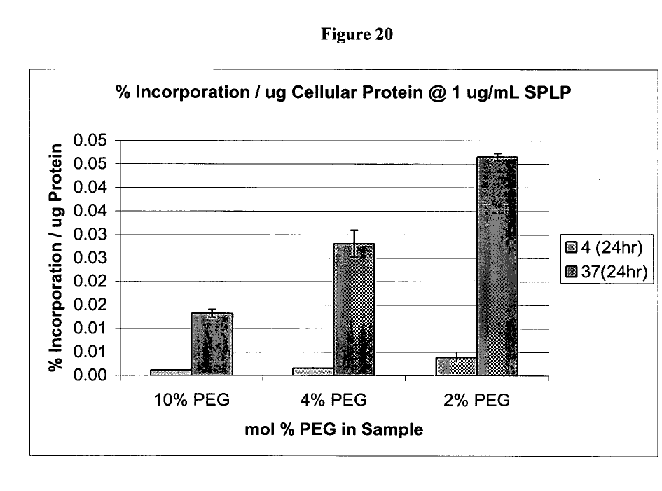

Uptake of SPLP occurred with greater efficiency at 37° C. and with decreasing amounts of PEG-C-DMA. The data is illustrated in FIG. 20.

### Example 7

### Biodistribution and Blood Clearance of SPLP Comprising PEG-DAA Conjugates

This example illustrates the biodistribution and blood clearance of SPLP comprising PEG-DAA conjugates. ³H-CHE -labeled SPLP comprising either PEG-C-DMA or PEG-C-DSA were administered intravenously to Neuro-2a tumor-bearing male A/J mice. SPLP were formulated as follows:

| | | Mol % |
|---|---|---|
| Group | Treatment | (DSPC:Chol:PEG-C-DMA:Cationic Lipid) |
| A | SPLP | 20:50:15:15 |
| | (15 mol % | |
| | PEG-C-DMA) | |
| B | SPLP | 20:55:10:15 |
| | (10 mol % | |
| | PEG-C-DMA) | |
| C | SPLP | 20:60:5:15 |
| | (5 mol % | |
| | PEG-C-DMA) | |

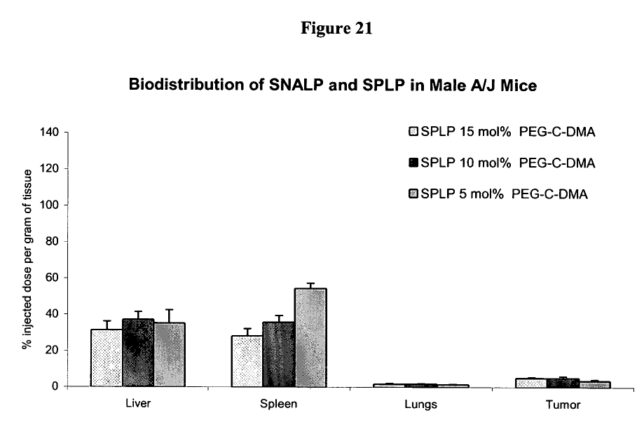

Biodistribution of SPLP in liver, spleen, lungs, and tumor was determined 48 hrs after SPLP administration. The results are shown in FIG. 21.

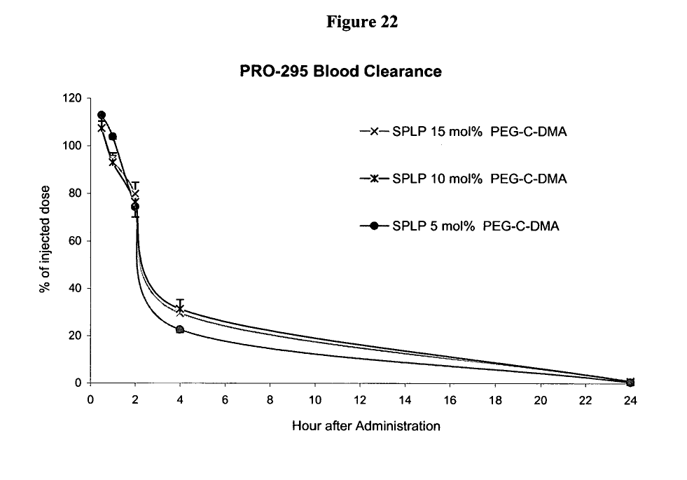

Blood clearance of SPLP was determined 1, 2, 4, and 24 hours after SPLP administration. The results are shown in FIG. 22.

### Example 8

### Biodistribution and Blood Clearance of SPLP and SNALP Comprising PEG-DAA Conjugates

This example illustrates the biodistribution and blood clearance of SPLP and SNALP comprising PEG-DAA conjugates. ³H-CHE -labeled SPLP or SNALP comprising either PEG-C-DMA or PEG-C-DSA were administered intravenously to Neuro-2a tumor-bearing male A/J mice. SPLP comprised an encapsulated plasmid encoding luciferase and SNALP comprised an encapsulated an anti-luciferase siRNA sequence. The SPLP and SNALP formulations all had the following lipid ratios: DSPC 20%: Cholesterol 55%: PEG-Lipid 10%: DODMA 15%.

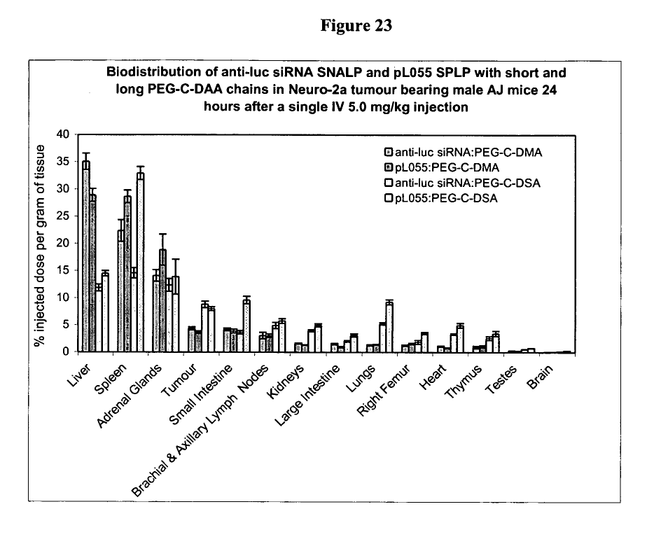

Biodistribution of SPLP or SNALP in liver, spleen, adrenal glands, tumor, small intestine, lymph nodes, kidneys, large intestine, femur, heart, thymus, testes, and brain was determined 24 hrs after administration of SPLP or SNALP. The results are shown in FIG. 23.

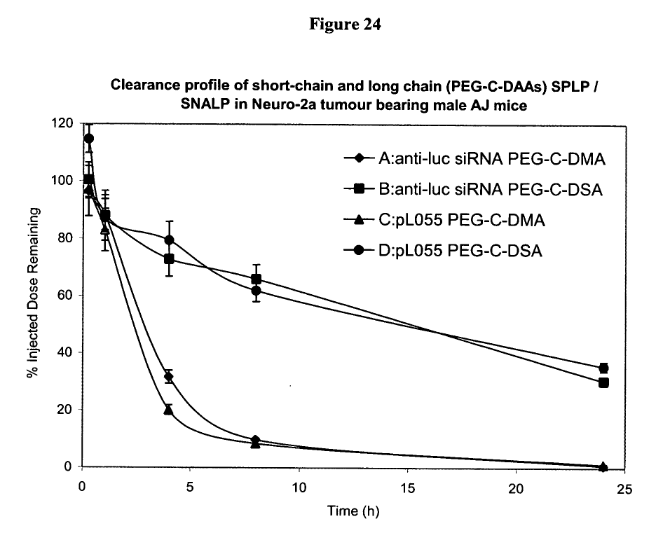

Blood clearance of SPLP and SNALP comprising PEG-C-DMA or PEG-C-DSA was determined 1, 2, 4, 8, and 24 hours after administration of the SPLP and SNALP. The results are shown in FIG. 24.

### Example 9

### Transfection of Cells by SPLP and pSPLP Comprising PEG-DAA Conjugates

This example describes three separate experiments conducted to assess gene expression in organs and tumors following in vivo transfection with various SPLP formulations encapsulating a plasmid encoding luciferase under the control of the CMV promoter.

The first experiment assessed luciferase gene expression in Neuro2A tumor bearing male A/J mice after intravenous administration of SPLP and pSPLP. Formulations comprising C14 and C18 PEG-C-DAAs were compared to the equivalent PEG-DAGs. The PEG moieties had a molecular weight of 2000 daltons. DODMA was used as the cationic lipid in the SPLP. Either POPG or DOP was used as the anionic lipid in the pSPLP. The SPLP and pSPLP were formulated as follows:

| | | Mol % |
|---|---|---|
| | Sample Description, (PEG-Lipid | (DSPC:Chol:PEG-Lipid:Charged |
| | type, Charged Lipid type) | Lipid) |
| A | SPLP (PEG-DSG, DODMA) | 20:50:15:15 |
| B | SPLP (PEG-DMG, DODMA) | 20:55:10:15 |
| C | SPLP (PEG-C-DSA, DODMA) | 20:60:5:15 |
| D | SPLP (PEG-C-DMA, DODMA) | 20:62.5:2.5:15 |
| E | pSPLP (PEG-C-DSA, POPG) | 20:55:10:15 |
| F | pSPLP (PEG-C-DSA, DOP) | 20:60:5:15 |
| G | pSPLP (PEG-DSG, POPG) | 20:62.5:2.5:15 |

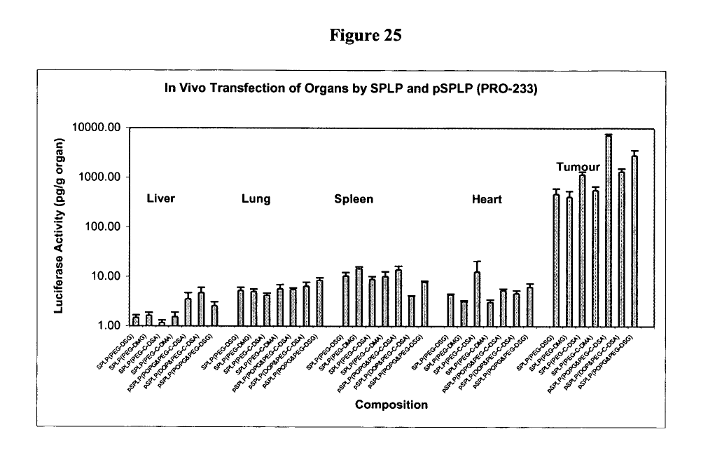

Luciferase gene expression was measured in liver, lung, spleen, heart, and tumors 48 hours after intravenous administration of SPLP and pSPLP. Luciferase expression was highest in tumors relative to other tissue types for all SPLP and pSPLP formulations tested. The results are shown in FIG. 25.

The second experiment assessed luciferase gene expression in Neuro2A tumor bearing male A/J mice after intravenous administration of SPLP comprising varying percentages (i.e., 15%, 10%, 5%, or 2.5%) of PEG-C-DMA.

| | Mol % |
|---|---|
| | (DSPC:Chol:PEG-C-DMA:DODMA) |
| A | 20:50:15:15 |
| B | 20:55:10:15 |
| C | 20:60:5:15 |
| D | 20:62.5:2.5:15 |

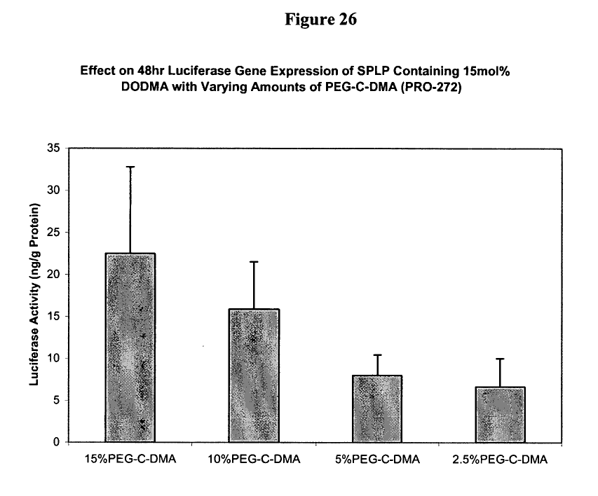

Luciferase expression in tumors was measured 48 hours after administration of SPLP. The results are shown in FIG. 26.

The third set of experiments assessed luciferase gene expression in Neuro2A tumor bearing male A/J mice after intravenous administration of SPLP comprising PEG-C-DMA conjugates with various sizes of PEG moieties (i.e., 2000 or 750 daltons).

| | Sample Description |
|---|---|
| A | SPLP-PEG₂₀₀₀-C-DMA |
| | (CHOL:DSPC:DODMA:PEG₂₀₀₀-C-DMA 55:20:15:10 mol %) |
| B | SPLP-PEG₇₅₀-C-DMA/DODMA |
| | (CHOL:DSPC:DODMA:PEG₇₅₀-C-DMA 55:20:15:10 mol %) |
| C | SPLP-High PEG₇₅₀-C-DMA |
| | (CHOL:DSPC:DODMA:PEG₇₅₀-C-DMA 50:20:15:15 mol %) |
| D | SPLP-DODAC (CHOL:DSPC:DODMA:PEG₂₀₀₀-C- |
| | DMA:DODAC 45:20:15:10:10 mol %) |
| | 0.35 mg/ml |

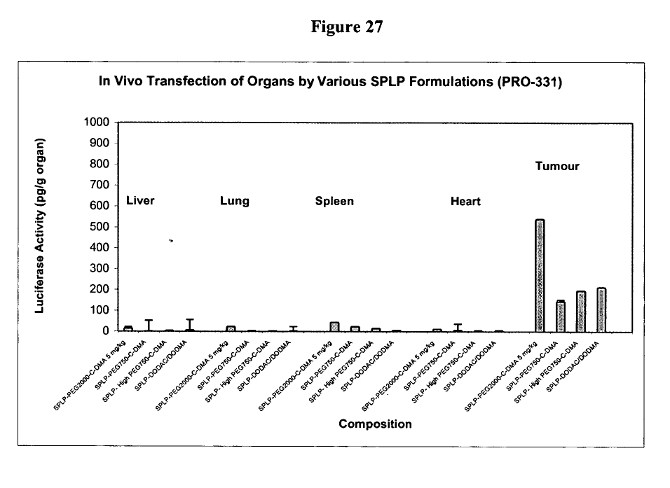

Luciferase gene expression was measured in liver, lung, spleen, heart, and tumors 48 hours after administration of SPLP. Luciferase expression was highest in tumors relative to other tissue types for all SPLP formulations tested. The results are shown in FIG. 27.

### Example 10

### In Vitro Silencing of Gene Expression with SNALPs Comprising PEG-DAA Conjugates

This example describes in vitro silencing of gene expression following delivery of SNALP encapsulating siRNA. Neuro2A-G cells expressing luciferase were contacted with SNALP formulations encapsulating anti-luciferase siRNA (i.e., siRNA comprising the following sequence: GAUUAUGUCCGGUUAUGUAUU and targeting the DNA sequence: GATTATGTCCGGTTATGTATT) for 48 hours in the presence or absence of chloroquine. The SNALP formulations contained varying amounts of PEG-C-DMA (C₁₄), i.e., 1%, 2%, 4%, or 10%. The cationic lipid was DODMA

| | | Mol % |
|---|---|---|
| Group | Treatment | (DSPC:Chol:PEG-C-DAA:DODMA) |
| A | PBS | — |
| B | Naked siRNA | — |
| C | SNALP (PEG-C-DMA) | 20:40:10:30 |
| D | SNALP (PEG-C-DMA) | 20:46:4:30 |
| E | SNALP (PEG-C-DMA) | 20:48:2:30 |
| F | SNALP (PEG-C-DMA) | 20:49:1:30 |

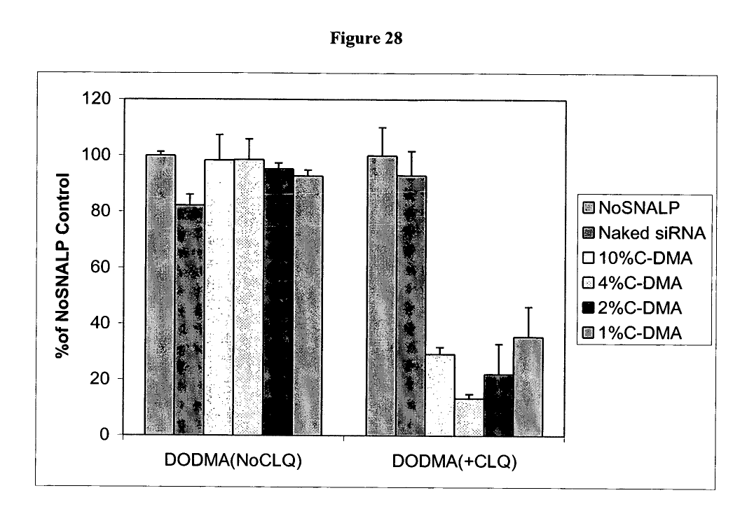

The results are shown in FIG. 28.

### Example 11

### In Vivo Silencing of Gene Expression with SNALPs Comprising PEG-DAA Conjugates

This example describes an experiment that demonstrates in vivo silencing of gene expression following administration of SNALP encapsulating siRNA.

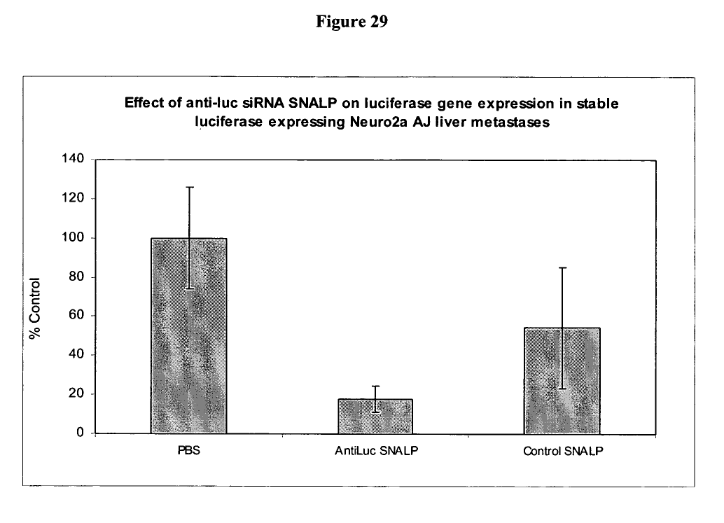

The experiment demonstrates that administration of SNALP encapsulating siRNA can silence gene expression in metastatic tumors. Neuro-2a tumor bearing male A/J mice expressing luciferase with metastatic liver tumors were treated with SNALPs comprising a PEG-DAA conjugate and encapsulating anti-luciferase siRNA (i.e., siRNA comprising the following sequence: GAUUAUGUCCGGUUAUGUAUU and targeting the DNA sequence: GATTATGTCCGGTTATGTATT). All SNALPs had the following formulation: DSPC 20%: Cholesterol 55%: PEG-C-DMA 10% : DODMA 15%. Mice received a single intravenous administration of SNALP. Luciferase expression in the tumors was determined 48 hours after SNALP injection. The results demonstrate that administration of SNALP can silence gene expression in vivo at a site distal to the site of SNALP administration. These results are shown in FIG. 29.

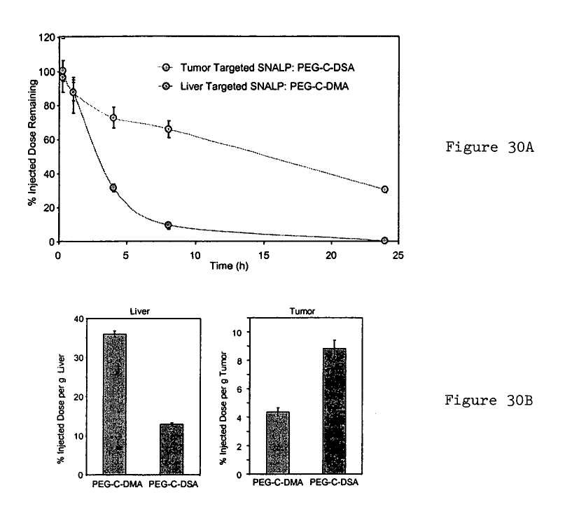

The above data, as well as FIG. 30, illustrates that SNALP encapsulating siRNA exhibit extended blood circulation times that are regulated by the PEG-lipid, and further illustrates that SNALP can be programmed to target specific disease sites including the liver and distal tumour.

It is to be understood that the above description is intended to be illustrative and not restrictive. Many embodiments will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all articles and references, including patent applications, patents and PCT publications, are incorporated herein by reference for all purposes.

What is claimed is:

1. A method of introducing a nucleic acid into a tumor cell, said method comprising contacting said tumor cell with a nucleic acid-lipid particle comprising a cationic lipid, a noncationic lipid, a PEG-lipid conjugate, and a nucleic acid, wherein the alkyl or acyl chains of the lipid portion of said PEG-lipid conjugate comprise from 12 to 20 carbon atoms.

2. The method in accordance with claim 1, wherein said cationic lipid is a member selected from the group consisting of: 1,2-DiLinoleyloxy-N,N-dimethylaminopropane (DLinDMA), 1,2-Dilinolenyloxy-N,N-dimethylaminopropane (DLenDMA), N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1 -(2,3-dioleyloxy)propyl)-N,N,N-trimethylammonium chloride (DOTMA), and N,N-dimethyl-2,3-dioleyloxy)propylamine (DODMA), and a mixture thereof.

3. The method in accordance with claim 1, wherein said noncationic lipid is a member selected from the group consisting of: (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl- phosphatidylethanolamine (POPE) and dioleoyl- phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), and 1,2-dielaidoyl-sn-glycero-3-phophoethanolamine (transDOPE).

4. The method in accordance with claim 1, wherein the alkyl or acyl chains of the lipid portion of said PEG-lipid conjugate comprise from 16 to 20 carbon atoms.

5. The method in accordance with claim 1, wherein said noncationic lipid is an anionic lipid.

6. The method in accordance with claim 1, wherein said noncationic lipid is a neutral lipid.

7. The method in accordance with claim 1, wherein said PEG-lipid is a member selected from the group consisting of a PEG-diacylglycerol (DAG), a PEG dialkyloxypropyl (DAA), a PEG-phospholipid, a PEG-ceramide, and combinations thereof.

8. The method in accordance with claim 1, wherein said PEG-lipid is a PEG dialkyloxypropyl (DAA) selected from the group consisting of: a PEG-dipalmityloxypropyl (C₁₆); a PEG-distearyloxypropyl (C₁₈); and a PEG-diicosyloxypropyl (C₂₀).

9. The method in accordance with claim 1, wherein said PEG-lipid is PEG-dialkyloxypropyl (DAA) having the following structure: 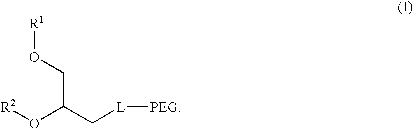
wherein:
R¹ and R² are independently selected and are alkyl groups having from about 16 to about 20 carbon atoms;
PEG is a polyethyleneglycol; and
L is a non-ester containing linker moiety.

10. The method in accordance with claim 1, wherein said PEG-lipid is PEG-diacylglycerol (DAG) selected from the group consisting of a PEG-dipalmitoylglycerol (C₁₆), a PEG-disterylglycerol (C₁₈) and a PEG-diicosylglycerol (C20).

11. The method in accordance with claim 1, wherein said PEG-lipid is PEG-ceramide (Cer) selected from the group consisting of PEG-ceramide (C₁₆), a PEG-ceramide (C₁₈) and PEG-ceramide (C₂₀).

12. The method in accordance with claim 1, wherein said cationic lipid comprises from about 5% to about 45% of the total lipid present in said particle.

13. The method in accordance with claim 1, wherein said cationic lipid comprises from about 5% to about 15% of the total lipid present in said particle.

14. The method in accordance with claim 1, wherein said cationic lipid comprises from about 30% to about 50% of the total lipid present in said particle.

15. The method in accordance with claim 1, wherein said cationic lipid comprises about 40% of the total lipid present in said particle.

16. The method in accordance with claim 1, wherein said noncationic lipid comprises from about 5% to about 90% of the total lipid present in said particle.

17. The method in accordance with claim 1, wherein said noncationic lipid comprises from about 20% to about 85% of the total lipid present in said particle.

18. The method in accordance with claim 1, wherein said PEG-lipid conjugate comprises from 1% to about 20% of the total lipid present in said particle.

19. The method in accordance with claim 1, wherein said PEG-lipid conjugate comprises from 2% to about 15% of the total lipid present in said particle.

20. The nucleic acid-lipid particle in accordance with claim 1, wherein said PEG-lipid conjugate comprises about 2% of the total lipid present in said particle.

21. The method in accordance with claim 1, wherein said noncationic lipid is DSPC.

22. The method in accordance with claim 1, wherein said nucleic acid-lipid particle further comprises cholesterol.

23. The method in accordance with claim 22, wherein the cholesterol comprises from about 0% to about 10% of the total lipid present in said particle.

24. The method in accordance with claim 22, wherein the cholesterol comprises from about 10% to about 60% of the total lipid present in said particle.

25. The method in accordance with claim 22, wherein the cholesterol comprises from about 20% to about 45% of the total lipid present in said particle.

26. The method in accordance with claim 1, wherein said nucleic acid is DNA.

27. The method in accordance with claim 1, wherein said nucleic acid is a plasmid.

28. The method in accordance with claim 1, wherein said nucleic acid is an antisense oligonucleotide.

29. The method in accordance with claim 1, wherein said nucleic acid is a ribozyme.

30. The method in accordance with claim 1, wherein said nucleic acid is a small interfering RNA (siRNA).

31. The method in accordance with claim 1, wherein said nucleic acid encodes a therapeutic product of interest.

32. The method in accordance with claim 31, wherein said therapeutic product of interest is a peptide or protein.

33. The method in accordance with claim 31, wherein said therapeutic product of interest is a small interfering RNA (siRNA).

34. The method in accordance with claim 1, wherein the nucleic acid in said nucleic acid-lipid particle is not substantially degraded after exposure of said particle to a nuclease at 37° C. for 20 minutes.

35. The method in accordance with claim 1, wherein the nucleic acid in said nucleic acid-lipid particle is not substantially degraded after incubation of said particle in serum at 37° C. for 30 minutes.

36. The method in accordance with claim 1, wherein the nucleic acid is fully encapsulated in said nucleic acid-lipid particle.

37. A method of introducing a nucleic acid to the lung of a mammal, said method comprising administering to said mammal a nucleic acid-lipid particle comprising a cationic lipid, a noncationic lipid, a PEG-lipid conjugate, and a nucleic acid, wherein the alkyl or acyl chains of the lipid portion of said PEG-lipid conjugate comprise from 16 to 20 carbon atoms.

38. The method in accordance with claim 37, wherein said PEG-lipid is a PEG dialkyloxypropyl (DAA) slected from the group consisting of a PEG-dipalmityloxypropyl (C₁₆), PEG-distearyloxypropyl (C₁₈), and a PEG-diicosyloxypropyl (C₂₀).

39. The method in accordance with claim 37, wherein said PEG-lipid is PEG-diacylglycerol (DAG) selected from the group consisting of: a PEG-dipalmitoylglycerol (C₁₆), and a PEG-disterylglycerol (C₁₈).

40. The method in accordance with claim 37, wherein said PEG-lipid is PEG-ceramide (Cer) selected from the group consisting of: PEG-ceramide (C₁₆), and a PEG-ceramide (C₁₈) and PEG-ceramide (C₂₀).

41. A method of introducing a nucleic acid to the liver of a mammal, said method comprising administering to said mammal a nucleic acid-lipid particle comprising a cationic lipid, a noncationic lipid, a PEG-lipid conjugate, and a nucleic acid, wherein the alkyl or acyl chains of the lipid portion of said PEG-lipid conjugate comprise from 8 to 14 carbon atoms.

42. The method in accordance with claim 41, wherein said PEG-lipid is as PEG dialkyloxypropyl (DAA) slected from the group consisting of a PEG-dilauryloxypropyl (C₁₂) and a PEG-dimyristyloxypropyl (C₁₄).

43. The method in accordance with claim 41, wherein said PEG-lipid is PEG-diacylglycerol (DAG) selected from the group consisting of: PEG-dilaurylglycerol (C₁₂) and a PEG-dimyristylglycerol (C₁₄).

44. The method in accordance with claim 41, wherein said PEG-lipid is PEG-ceramide (Cer) selected from the group consisting of: PEG-ceramide (C₁₂) and a PEG-ceramide (C₁₄).

45. A method of introducing a nucleic acid to the spleen of a mammal, said method comprising administering to said mammal a nucleic acid-lipid particle comprising a cationic lipid, a noncationic lipid, a PEG-lipid conjugate, and a nucleic acid, wherein the alkyl or acyl chains of the lipid portion of said PEG-lipid conjugate comprise from 8 to 14 carbon atoms.

46. The method in accordance with claim 45, wherein said PEG-lipid is a PEG dialkyloxypropyl (DAA) slected from the group consisting of a PEG-dilauryloxypropyl (C₁₂) and a PEG-dimyristyloxypropyl (C₁₄).

47. The method in accordance with claim 45, wherein said PEG-lipid is PEG-diacylglycerol (DAG) selected from the group consisting of: PEG-dilaurylglycerol (C₁₂) and a PEG-dimyristylglycerol (C₁₄).

48. The method in accordance with claim 45, wherein said PEG-lipid is PEG-ceramide (Cer) selected from the group consisting of selected from the group consisting of PEG-ceramide (C₁₂) and a PEG-ceramide (C₁₄).
